SEQUENCE LISTING (100 sequences)

SEQ ID NO: 1 - protein, 5 aa, Homo sapiens
SYGMH

SEQ ID NO: 2 - protein, 5 aa, Homo sapiens
DYGMS

SEQ ID NO: 3 - protein, 6 aa, Homo sapiens
SSNWWS

SEQ ID NO: 4 - protein, 5 aa, Homo sapiens
NYGMS

SEQ ID NO: 5 - protein, 5 aa, Homo sapiens
SYWIG

SEQ ID NO: 6 - protein, 7 aa, Homo sapiens
TSGVGVG

SEQ ID NO: 7 - protein, 5 aa, Homo sapiens
TYYRS

SEQ ID NO: 8 - protein, 5 aa, Homo sapiens
SYSLK

SEQ ID NO: 9 - protein, 7 aa, Homo sapiens
TGGVGVG

SEQ ID NO: 10 - protein, 5 aa, Homo sapiens
EVFMH

SEQ ID NO: 11 - protein, 17 aa, Homo sapiens
FIWYDGSNKNYADSVKG

SEQ ID NO: 12 - protein, 17 aa, Homo sapiens
GLSWNGGNTYYADSVKG

SEQ ID NO: 13 - protein, 16 aa, Homo sapiens
EIYHSGSTSYNPSLKS

SEQ ID NO: 14 - protein, 17 aa, Homo sapiens
GINWNGGSTYYADSVRG

SEQ ID NO: 15 - protein, 17 aa, Homo sapiens
IIYPGDSDTRYSPSFQG

SEQ ID NO: 16 - protein, 16 aa, Homo sapiens
LIYWNDDKRYRPSLKN

SEQ ID NO: 17 - protein, 16 aa, Homo sapiens
YIYYSGNTNYNPSLKS

SEQ ID NO: 18 - protein, 17 aa, Homo sapiens
FVWYDGSNQNYADSVKG

SEQ ID NO: 19 - protein, 17 aa, Homo sapiens
SISSSSGYIYYADSVKG

SEQ ID NO: 20 - protein, 16 aa, Homo sapiens
LIYWNDDKRYSPSLKS

SEQ ID NO: 21 - protein, 17 aa, Homo sapiens
GFDPEDGETIYAQKFQG

SEQ ID NO: 22 - protein, 12 aa, Homo sapiens
DRYSSGWDVFDY

SEQ ID NO: 23 - protein, 13 aa, Homo sapiens
GSRYNWNYDAFDI

SEQ ID NO: 24 - protein, 9 aa, Homo sapiens
NDWGFPLDC

SEQ ID NO: 25 - protein, 13 aa, Homo sapiens
GSRYNWSYDAFDI

SEQ ID NO: 26 - protein, 9 aa, Homo sapiens
RGWVNYFDY

SEQ ID NO: 27 - protein, 13 aa, Homo sapiens
YGVTAPFYNWFDP

SEQ ID NO: 28 - protein, 10 aa, Homo sapiens
AQYGEEAFDI

SEQ ID NO: 29 - protein, 18 aa, Homo sapiens
AVSTIAARPGYYYYYMDV

SEQ ID NO: 30 - protein, 13 aa, Homo sapiens
SGLAAPFYNWFDP

SEQ ID NO: 31 - protein, 9 aa, Homo sapiens
GWGSRYFDY

SEQ ID NO: 32 - protein, 11 aa, Homo sapiens
RASQDISSWLA

SEQ ID NO: 33 - protein, 11 aa, Homo sapiens
RASQGIRNALG

SEQ ID NO: 34 - protein, 11 aa, Homo sapiens
RASQSISSYLN

SEQ ID NO: 35 - protein, 11 aa, Homo sapiens
RASQGVRNALG

SEQ ID NO: 36 - protein, 11 aa, Homo sapiens
RASQGISNWLG

SEQ ID NO: 37 - protein, 11 aa, Homo sapiens
RASQSITSYLN

SEQ ID NO: 38 - protein, 11 aa, Homo sapiens
RASQSITTYLN

SEQ ID NO: 39 - protein, 11 aa, Homo sapiens
RASQGISSWLA

SEQ ID NO: 40 - protein, 11 aa, Homo sapiens
RASQDISTWFA

SEQ ID NO: 41 - protein, 11 aa, Homo sapiens
RASQSISNYLN

SEQ ID NO: 42 - protein, 17 aa, Homo sapiens
KSSQSVLYSSNNKNYLA

SEQ ID NO: 43 - protein, 7 aa, Homo sapiens
AASSLQS

SEQ ID NO: 44 - protein, 7 aa, Homo sapiens
AASNLQS

SEQ ID NO: 45 - protein, 7 aa, Homo sapiens
AASRLQT

SEQ ID NO: 46 - protein, 7 aa, Homo sapiens
AASTLRS

SEQ ID NO: 47 - protein, 7 aa, Homo sapiens
WASTRES

SEQ ID NO: 48 - protein, 9 aa, Homo sapiens
QQAKSFPPT

SEQ ID NO: 49 - protein, 9 aa, Homo sapiens
LQHNSYPFT

SEQ ID NO: 50 - protein, 9 aa, Homo sapiens
QQSHNIPIT

SEQ ID NO: 51 - protein, 9 aa, Homo sapiens
QQANSFPLT

SEQ ID NO: 52 - protein, 8 aa, Homo sapiens
QRSYSTLT

SEQ ID NO: 53 - protein, 9 aa, Homo sapiens
QQSYNTPPT

SEQ ID NO: 54 - protein, 9 aa, Homo sapiens
HQYYSTPLT

SEQ ID NO: 55 - protein, 121 aa, Homo sapiens
QVQLVESGGGVVQPGRSLRLSCVASGFTFSSYGMHWVRQAPGKGLEWVTFIWYDGSNKNYADSVKGRFTISRDNSKNTLNLQMNSLRVEDTAVYYCARDRYSSGWDVFDYWGQGTLVTVSS

SEQ ID NO: 56 - protein, 122 aa, Homo sapiens
EVQLVESGGGVVRPGGSLRLSCAASGFTFDDYGMSWVRQAPGKGLEWVSGLSWNGGNTYYADSVKGRFTISRDNAKNSLYLQMNSLRAEDTALYYCARGSRYNWNYDAFDIWGQGTLVTVSS

SEQ ID NO: 57 - protein, 118 aa, Homo sapiens
QVQLQESGPGLVKPSGTLSLTCAVSGGSISSSNWWSWVRQPPGKGLEWIGEIYHSGSTSYNPSLKSRVTISVDKSKNQFSLKLSSVTAADTAVYYCARNDWGFPLDCWGQGTLVTVSS

SEQ ID NO: 58 - protein, 122 aa, Homo sapiens
EVQLVESGGGVVRPGGSLRLSCAASGFTFDNYGMSWVRQAPGKGLEWVSGINWNGGSTYYADSVRGRFTISRDNAKNSLYLQMNSLRAEDTALYYCARGSRYNWSYDAFDIWGQGTLVTVSS

SEQ ID NO: 59 - protein, 118 aa, Homo sapiens
EVQLVQSGTEVKKPGESLKISCKDSGYRFSSYWIGWVRQMPGKGLEWMGIIYPGDSDTRYSPSFQGQVTISADKSISTAYLQWRSLKASDTAMYYCARRGWVNYFDYWGQGTLVTVSS

SEQ ID NO: 60 - protein, 123 aa, Homo sapiens
QITLKESGPTLVKPTQTLTLTCTFSGFSLSTSGVGVGWIRQPPGKALEWLALIYWNDDKRYRPSLKNRLTITKDTSKNQVVLTMTNMDPVDTATYYCTHYGVTAPFYNWFDPWGQGTLVTVSS

SEQ ID NO: 61 - protein, 118 aa, Homo sapiens
QVQLQESGPGLVKPSETLSLTCTVSGGSINTYYRSWIRQPPGKGLEWIGYIYYSGNTNYNPSLKSRVTISADTSKNQFSLNLISVTAADTAMYYCARAQYGEEAFDIWGQGTLVTVSS

SEQ ID NO: 62 - protein, 121 aa, Homo sapiens
QVQLVESGGGVVQPGRSLRLSCAASGFNFSSYGMHWVRQAPGKGLEWVAFVWYDGSNQNYADSVKGRFTISRDNSNNTLYLQMNSLRAEDTAVYYCARDRYSSGWDVFDYWGQGTLVTVSS

SEQ ID NO: 63 - protein, 127 aa, Homo sapiens
EVQLVESGGGLVKSGGSLRLSCAASGFTFSSYSLKWVRQAPGKGLEWVSSISSSSGYIYYADSVKGRFTISRDNAKNSLYLQMNSLRAEDTAMYYCARAVSTIAARPGYYYYYMDVWGKGTLVTVSS

SEQ ID NO: 64 - protein, 123 aa, Homo sapiens
QITLKESGPTLVKPTQTLTLTCTFSGISLSTGGVGVGWIRQPPGKALEWLSLIYWNDDKRYSPSLKSRLTITKDSSKNQVVLTMTNMDPVDTATYYCAHSGLAAPFYNWFDPWGQGTLVTVSS

SEQ ID NO: 65 - protein, 118 aa, Homo sapiens
QVQLVQSGAEVKKPGASVKVSCKVSGYTLTEVFMHWVRQAPGKGLEWMGGFDPEDGETIYAQKFQGRVTMTEDTFIDTAYMDLSSLRSEDTAVYYCAIGWGSRYFDYWGQGTLVTVSS

SEQ ID NO: 66 - protein, 107 aa, Homo sapiens
DIQMTQSPSSVSASVGDRVTITCRASQDISSWLAWYQQQPGKAPKLQIYAASSLQSGVPSRFSGSGSGTDFTLTISSLQPEDFATYYCQQAKSFPPTFGQGTKVEIK

SEQ ID NO: 67 - protein, 107 aa, Homo sapiens
DIQMTQSPSSLSASIGDRVTITCRASQGIRNALGWYQQKPGKAPKRLIYAASNLQSGVPSRFSGSGSGTEFTLTISSLQPEDFATYYCLQHNSYPFTFGPGTKVEIK

SEQ ID NO: 68 - protein, 107 aa, Homo sapiens
DIQMTQSPSSLSASVGDRVTITCRASQSISSYLNWYQQKPGKAPKLLIYAASSLQSGVPSRFSGSGSGTHFTLTISSLQPEDFATYYCQQSHNIPITFGQGTRLEIK

SEQ ID NO: 69 - protein, 107 aa, Homo sapiens
DIQMTQSPSSLSASVGDRVTITCRASQGVRNALGWYQQKPGKAPKRLIYAASSLQSGVPSRFSGSGSGTEFTLTISSLQPEDFATYYCLQHNSYPFTFGPGTKVEIK

SEQ ID NO: 70 - protein, 107 aa, Homo sapiens
DIQMTQSPSSVSASVGDRVTITCRASQGISNWLGWYQQKPGKAPKLLIYAASSLQSGVPSRFSGSGSGTDFTLTISSLQPEDFATYYCQQANSFPLTFGGGTKVEIK

SEQ ID NO: 71 - protein, 106 aa, Homo sapiens
DIQMTQSPSSLSASVGDRVTITCRASQSITSYLNWYQQKPGKAPKLLIYAASSLQSGVPSRFSGSGSGTDFTLTISSLQPEDFATYYCQRSYSTLTFGGGTKVEIK

SEQ ID NO: 72 - protein, 107 aa, Homo sapiens
DIQMTQSPSSLSASVGDRVTITCRASQSITTYLNWYQQKPGKAPQLLIFAASRLQTGVPSRFSGSGSGTDFTLTISSLLPGDFATYYCQQSYNTPPTFGGGTKVEIK

SEQ ID NO: 73 - protein, 107 aa, Homo sapiens
DIQMTQSPSSVSASVGDRVTITCRASQGISSWLAWYQQKPGKAPKLLIFAASSLQSGVPSRFSGSGSGTGFTLTISSLQPEDFATYYCQQAKSFPPTFGQGTKVEIK

SEQ ID NO: 74 - protein, 107 aa, Homo sapiens
DIQMTQSPSSVSASIGDRVTITCRASQDISTWFAWYQQKPGKAPKLLIYAASTLRSGVPSRFSGSGSGTDFTLTINSLQPEDFATYYCQQANSFPLTFGGGTKVEIK

SEQ ID NO: 75 - protein, 106 aa, Homo sapiens
DIQMTQSPSSLSASVGDRVTITCRASQSISNYLNWYQLKPGNAPKLLIFAASSLQSGVPSRFSGSGSGTDFTLTISSLQPEDFATYFCQRSYSTLTFGGGTKVEIK

SEQ ID NO: 76 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCHQYYSTPLTFGGGTKVEIK

SEQ ID NO: 77 - protein, 451 aa, Homo sapiens
QVQLVESGGGVVQPGRSLRLSCVASGFTFSSYGMHWVRQAPGKGLEWVTFIWYDGSNKNYADSVKGRFTISRDNSKNTLNLQMNSLRVEDTAVYYCARDRYSSGWDVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 214 aa, Homo sapiens
DIQMTQSPSSVSASVGDRVTITCRASQDISSWLAWYQQQPGKAPKLQIYAASSLQSGVPSRFSGSGSGTDFTLTISSLQPEDFATYYCQQAKSFPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 452 aa, Homo sapiens
EVQLVESGGGVVRPGGSLRLSCAASGFTFDDYGMSWVRQAPGKGLEWVSGLSWNGGNTYYADSVKGRFTISRDNAKNSLYLQMNSLRAEDTALYYCARGSRYNWNYDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 214 aa, Homo sapiens
DIQMTQSPSSLSASIGDRVTITCRASQGIRNALGWYQQKPGKAPKRLIYAASNLQSGVPSRFSGSGSGTEFTLTISSLQPEDFATYYCLQHNSYPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 448 aa, Homo sapiens
QVQLQESGPGLVKPSGTLSLTCAVSGGSISSSNWWSWVRQPPGKGLEWIGEIYHSGSTSYNPSLKSRVTISVDKSKNQFSLKLSSVTAADTAVYYCARNDWGFPLDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 214 aa, Homo sapiens
DIQMTQSPSSLSASVGDRVTITCRASQSISSYLNWYQQKPGKAPKLLIYAASSLQSGVPSRFSGSGSGTHFTLTISSLQPEDFATYYCQQSHNIPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 452 aa, Homo sapiens
EVQLVESGGGVVRPGGSLRLSCAASGFTFDNYGMSWVRQAPGKGLEWVSGINWNGGSTYYADSVRGRFTISRDNAKNSLYLQMNSLRAEDTALYYCARGSRYNWSYDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 214 aa, Homo sapiens
DIQMTQSPSSLSASVGDRVTITCRASQGVRNALGWYQQKPGKAPKRLIYAASSLQSGVPSRFSGSGSGTEFTLTISSLQPEDFATYYCLQHNSYPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 448 aa, Homo sapiens
EVQLVQSGTEVKKPGESLKISCKDSGYRFSSYWIGWVRQMPGKGLEWMGIIYPGDSDTRYSPSFQGQVTISADKSISTAYLQWRSLKASDTAMYYCARRGWVNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 214 aa, Homo sapiens
DIQMTQSPSSVSASVGDRVTITCRASQGISNWLGWYQQKPGKAPKLLIYAASSLQSGVPSRFSGSGSGTDFTLTISSLQPEDFATYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 453 aa, Homo sapiens
QITLKESGPTLVKPTQTLTLTCTFSGFSLSTSGVGVGWIRQPPGKALEWLALIYWNDDKRYRPSLKNRLTITKDTSKNQVVLTMTNMDPVDTATYYCTHYGVTAPFYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 213 aa, Homo sapiens
DIQMTQSPSSLSASVGDRVTITCRASQSITSYLNWYQQKPGKAPKLLIYAASSLQSGVPSRFSGSGSGTDFTLTISSLQPEDFATYYCQRSYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 448 aa, Homo sapiens
QVQLQESGPGLVKPSETLSLTCTVSGGSINTYYRSWIRQPPGKGLEWIGYIYYSGNTNYNPSLKSRVTISADTSKNQFSLNLISVTAADTAMYYCARAQYGEEAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 214 aa, Homo sapiens
DIQMTQSPSSLSASVGDRVTITCRASQSITTYLNWYQQKPGKAPQLLIFAASRLQTGVPSRFSGSGSGTDFTLTISSLLPGDFATYYCQQSYNTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 451 aa, Homo sapiens
QVQLVESGGGVVQPGRSLRLSCAASGFNFSSYGMHWVRQAPGKGLEWVAFVWYDGSNQNYADSVKGRFTISRDNSNNTLYLQMNSLRAEDTAVYYCARDRYSSGWDVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 214 aa, Homo sapiens
DIQMTQSPSSVSASVGDRVTITCRASQGISSWLAWYQQKPGKAPKLLIFAASSLQSGVPSRFSGSGSGTGFTLTISSLQPEDFATYYCQQAKSFPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 457 aa, Homo sapiens
EVQLVESGGGLVKSGGSLRLSCAASGFTFSSYSLKWVRQAPGKGLEWVSSISSSSGYIYYADSVKGRFTISRDNAKNSLYLQMNSLRAEDTAMYYCARAVSTIAARPGYYYYYMDVWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 214 aa, Homo sapiens
DIQMTQSPSSVSASIGDRVTITCRASQDISTWFAWYQQKPGKAPKLLIYAASTLRSGVPSRFSGSGSGTDFTLTINSLQPEDFATYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 453 aa, Homo sapiens
QITLKESGPTLVKPTQTLTLTCTFSGISLSTGGVGVGWIRQPPGKALEWLSLIYWNDDKRYSPSLKSRLTITKDSSKNQVVLTMTNMDPVDTATYYCAHSGLAAPFYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 213 aa, Homo sapiens
DIQMTQSPSSLSASVGDRVTITCRASQSISNYLNWYQLKPGNAPKLLIFAASSLQSGVPSRFSGSGSGTDFTLTISSLQPEDFATYFCQRSYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 448 aa, Homo sapiens
QVQLVQSGAEVKKPGASVKVSCKVSGYTLTEVFMHWVRQAPGKGLEWMGGFDPEDGETIYAQKFQGRVTMTEDTFIDTAYMDLSSLRSEDTAVYYCAIGWGSRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 220 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCHQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 107 aa, Homo sapiens [sequence omitted]

(19) United States
(12) Patent Application Publication
(10) Pub. No.: US 2026/0232828 A1
(43) Pub. Date: Aug. 13, 2026

(54) LIGAND-CYTOTOXICITY DRUG CONJUGATES AND PHARMACEUTICAL USES THEREOF

(71) Applicants: HANSOH BIO LLC, Rockville, MD (US); SHANGHAI HANSOH BIOMEDICAL CO., LTD., Shanghai (CN); CHANGZHOU HANSOH PHARMACEUTICAL CO., LTD., Changzhou (CN)

(72) Inventors: Qing LI, Rockville, MD (US); Ningyun ZHOU, Rockville, MD (US)

(21) Appl. No.: 19/655,544

(22) Filed: Apr. 22, 2026

Related U.S. Application Data
(63) Continuation of application No. PCT/CN2024/126637, filed on Oct. 23, 2024.
(60) Provisional application No. 63/592,304, filed on Oct. 23, 2023.

(51) Int. Cl. A61K 47/68; A61K 39/00; A61P 35/00

(52) U.S. Cl. CPC A61K 47/68037; A61K 47/6849; A61K 47/6889; A61P 35/00; A61K 2039/505

(57) ABSTRACT

To provide an antitumor drug having an excellent therapeutic effect, which is excellent in terms of antitumor effect and safety. Provided is an anti-cadherin 17 antibody comprising a heavy chain variable region and a light chain variable region, a drug conjugate thereof, and a composition containing the anti-cadherin 17 antibody or the drug conjugate thereof, and an application thereof as a drug.

### CROSS-REFERENCE TO RELATED APPLICATIONS

This application is a continuation of International Patent Application No. PCT/CN2024/126637, filed on Oct. 23, 2024, which claims priority to and the benefit of U.S. Provisional Patent Application No. 63/592,304, filed on Oct. 23, 2023. The disclosure of each of the foregoing applications is incorporated by reference herein in its entirety.

### SEQUENCE LISTING

The Sequence Listing XML associated with this application is provided electronically in XML format and is hereby incorporated by reference into the specification. The name of the XML file containing the Sequence Listing XML is “30006-013753-US0-SL.xml”. The XML file is 96,931 bytes, created on Apr. 22, 2026, and is being submitted electronically via USPTO Patent Center.

### FIELD OF THE INVENTION

The present invention relates to a novel cadherin 17 antibody or functional fragments thereof, comprising engineered heavy chains and light chains. The present invention further relates to conjugates of the improved cadherin 17 antibodies with small molecule drugs. The present invention further relates to the use of the antibody and its conjugates in the manufacture of drugs for treating cancers.

### BACKGROUND ART

Cadherin 17 (CDH17) is a cell surface marker belonging to cadherin superfamily with a unique biological structure. It has 7 extracellular cadherin repeats compared to the classic 5 repeats cadherin proteins and has a very short intracellular region of 20 amino acid residues lacking the conserved intracellular domain. (Berndorff et al. J Cell Biol. 1994, 125(6):1353-1369). Although the biological function of CDH17 has not been fully explored yet, it has been reported that CDH17 can regulate water resorption in a Ca²⁺-dependent manner. (Ahl et al. Biol. Med. Model. 2011, 8(18)) CDH17 also involves in maintaining tissue integrity by interacting with integrin within the extracellular tight junctions. It mainly expresses in the human gastrointestinal (GI) tract and pancreas.

CDH17 has been reported to be highly expressed in tumors including colorectal, gastric, and pancreatic tumors in both DNA and protein levels (Takamura et al. Med Mol Morphol. 2013, 46:1-7). It plays an important role in regulating cancer metastasis and tumor growth. There are several signaling pathways involved in the CDH17 manipulated tumor activities. One of the most important mechanisms related to CDH17-integrin interaction. It has been proved that the RDG motif of CDH17 binds to α2β1 integrin which induces the β1 integrin activation leading to increase cancer cell proliferation and adhesion (Bartomome et al. J Biol Chem. 2014, 289(50):34801-34914). It has also been shown that CDH17 regulates the cancer invasion through Wnt/β-catenin signaling (Qiu et al. PloS one 2013, 8(3)) and NFκB signaling pathway (Wang et al. Cancer biology & therapy. 2013, 14(3): 262-270) in GI cancers. The restricted expression in normal tissue and high expression in various cancers makes CDH17 a good cancer target.

There are several CDH17-targeted antibody-based drugs that have been studied in the field. Two bispecific antibody drugs BI905711 (Boehringer Ingelheim) and ARB202 (Arbele) are in phase I trials and other anti-CDH17 CAR (Chimeric antigen receptor) and monoclonal drugs are in preclinical trials. However, no anti-CDH17 antibody-drug conjugate (ADC) has been reported yet. This invention presents the first-in-class anti-CDH17 ADCs in the field.

Antibody-drug conjugates (ADC) represent a new class of therapeutics comprising an antibody conjugated to a cytotoxic drug via a chemical linker. The therapeutic concept of ADCs is to combine the binding capabilities of an antibody with a drug, where the antibody is used to deliver the drug to a tumor cell by means of binding to a target surface antigen.

Accordingly, there remains a need in the art for anti-CDH17 antibodies and ADCs that can be used for therapeutic purposes in the treatment of CDH17 expression cancers. The conjugates have better biological activity, stability, homogeneity, and reduced toxic side effects.

### SUMMARY OF THE INVENTION

The invention provides anti-CDH17 antibodies and antibody-drug conjugates and methods of using the same. The anti-cancer efficacy of antibody-drug conjugates is thought to rely on their uptake by cancer cells expressing the surface antigen, so the insufficient internalization of CDH17-targeting monoclonal antibody is an urgent problem to be solved. In general ADC development needs to take into account all these key components, including the selection of target antigen, antibody, toxin drug, as well as linker.

The technical problem to be solved by the present invention is to develop the advanced anti-CDH17 antibody-drug conjugate which show very strong antitumor effects in cancers. The antitumor effects may be due to higher DAR and robust endocytosis.

Specifically, the present invention encompasses the following aspects:

The present disclosure relates to an antibody-drug conjugate or the pharmaceutically acceptable salt or solvate thereof and pharmaceutical use thereof, wherein the antibody-drug conjugate or the pharmaceutically acceptable salt or solvate thereof comprises an anti-CDH17 antibody or an antigen-binding fragment thereof conjugated to a toxin drug optionally by a linker.

The present disclosure relates to an antibody-drug conjugate or the pharmaceutically acceptable salt or solvate thereof and pharmaceutical use thereof, wherein the antibody-drug conjugate or the pharmaceutically acceptable salt or solvate thereof comprises an anti-CDH17 antibody or an antigen-binding fragment thereof conjugated to a toxin drug optionally by a linker, wherein the anti-CDH17 antibody or the antigen-binding fragment thereof comprises a heavy chain variable region and a light chain variable region of the antibody, wherein: a) a heavy chain variable region comprises HCDR1, HCDR2 and HCDR3 having the amino acid sequences as shown in SEQ ID NO: 01, SEQ ID NO: 11 and SEQ ID NO: 22, respectively; and a light chain variable region comprises LCDR1, LCDR2 and LCDR3 having the amino acid sequences as shown in SEQ ID NO: 32, SEQ ID NO: 43 and SEQ ID NO: 48, respectively; or b) a heavy chain variable region comprises HCDR1, HCDR2 and HCDR3 having the amino acid sequences as shown in SEQ ID NO: 02, SEQ ID NO: 12 and SEQ ID NO:23, respectively; and a light chain variable region sequence comprises LCDR1, LCDR2 and LCDR3 having the amino acid sequences as shown in SEQ ID NO: 33, SEQ ID NO: 44 and SEQ ID NO: 49, respectively; or c) a heavy chain variable region comprises HCDR1, HCDR2 and HCDR3 having the amino acid sequences as shown in SEQ ID NO: 03, SEQ ID NO: 13 and SEQ ID NO: 24, respectively; and a light chain variable region comprises LCDR1, LCDR2 and LCDR3 having the amino acid sequences as shown in SEQ ID NO: 34, SEQ ID NO: 43 and SEQ ID NO: 50, respectively; or d) a heavy chain variable region comprises HCDR1, HCDR2 and HCDR3 having the amino acid sequences as shown in SEQ ID NO: 04, SEQ ID NO: 14 and SEQ ID NO: 25, respectively; and a light chain variable region comprises LCDR1, LCDR2 and LCDR3 having the amino acid sequences as shown in SEQ ID NO: 35, SEQ ID NO: 43 and SEQ ID NO: 49, respectively; or e) a heavy chain variable region comprises HCDR1, HCDR2 and HCDR3 having the amino acid sequences as shown in SEQ ID NO: 05, SEQ ID NO: 15 and SEQ ID NO: 26, respectively; and a light chain variable region comprises LCDR1, LCDR2 and LCDR3 having the amino acid sequences as shown in SEQ ID NO: 36, SEQ ID NO: 43 and SEQ ID NO: 51, respectively; or f) a heavy chain variable region comprises HCDR1, HCDR2 and HCDR3 having the amino acid sequences as shown in SEQ ID NO: 06, SEQ ID NO: 16 and SEQ ID NO: 27, respectively; and a light chain variable region comprises LCDR1, LCDR2 and LCDR3 having the amino acid sequences as shown in SEQ ID NO: 37, SEQ ID NO: 43 and SEQ ID NO: 52, respectively; or g) a heavy chain variable region comprises HCDR1, HCDR2 and HCDR3 having the amino acid sequences as shown in SEQ ID NO: 07, SEQ ID NO: 17 and SEQ ID NO: 28, respectively; and a light chain variable region comprises LCDR1, LCDR2 and LCDR3 having the amino acid sequences as shown in SEQ ID NO: 38, SEQ ID NO: 45 and SEQ ID NO: 53, respectively; or h) a heavy chain variable region comprises HCDR1, HCDR2 and HCDR3 having the amino acid sequences as shown in SEQ ID NO: 01, SEQ ID NO: 18 and SEQ ID NO: 22, respectively; and a light chain variable region comprises LCDR1, LCDR2 and LCDR3 having the amino acid sequences as shown in SEQ ID NO: 39, SEQ ID NO: 43 and SEQ ID NO: 48, respectively; or i) a heavy chain variable region comprises HCDR1, HCDR2 and HCDR3 having the amino acid sequences as shown in SEQ ID NO: 08, SEQ ID NO: 19 and SEQ ID NO:29, respectively; and a light chain variable region comprises LCDR1, LCDR2 and LCDR3 having the amino acid sequences as shown in SEQ ID NO: 40, SEQ ID NO: 46 and SEQ ID NO: 51, respectively; or j) a heavy chain variable region comprises HCDR1, HCDR2 and HCDR3 having the amino acid sequences as shown in SEQ ID NO: 09, SEQ ID NO:20 and SEQ ID NO: 30, respectively; and a light chain variable region comprises LCDR1, LCDR2 and LCDR3 having the amino acid sequences as shown in SEQ ID NO: 41, SEQ ID NO: 43 and SEQ ID NO: 52, respectively; or k) a heavy chain variable region comprises HCDR1, HCDR2 and HCDR3 having the amino acid sequences as shown in SEQ ID NO: 10, SEQ ID NO:21 and SEQ ID NO: 31, respectively; and a light chain variable region comprises LCDR1, LCDR2 and LCDR3 having the amino acid sequences as shown in SEQ ID NO: 42, SEQ ID NO: 47 and SEQ ID NO: 54, respectively.

In some embodiments of the present disclosure, the anti-CDH17 antibody or antigen-binding fragment thereof according to any one of the aforementioned embodiments in the antibody-drug conjugate or the pharmaceutically acceptable salt or solvate thereof is a monoclonal antibody or antigen-binding fragment thereof, a polyclonal antibody or antigen-binding fragment thereof, a multi-specific antibody or antigen-binding fragment thereof, a murine antibody or antigen-binding fragment thereof, a chimeric antibody or antigen-binding fragment thereof, a humanized antibody or antigen-binding fragment thereof, a recombinant antibody or antigen-binding fragment thereof, a human antibody or antigen-binding fragment thereof; preferably, the multi-specific antibody is a bispecific antibody, a tri-specific antibody or a tetra-specific antibody.

In some embodiments of the present disclosure, the anti-CDH17 antibody or the antigen-binding fragment thereof according to any one of the aforementioned embodiments in the antibody-drug conjugate or the pharmaceutically acceptable salt or solvate thereof comprises a heavy chain variable region comprising an amino acid sequence selected from: SEQ ID NOs: 55-65, or sequence having at least 80%, 85%, 90%, 95% or 99% sequence identity therewith; and/or a light chain variable region comprising an amino acid sequence selected from: SEQ ID NOs: 66-76, or sequence having at least 80%, 85%, 90%, 95% or 99% sequence identity therewith.

In some embodiments of the present disclosure, the anti-CDH17 antibody or the antigen-binding fragment thereof according to any one of the aforementioned embodiments in the antibody-drug conjugate or the pharmaceutically acceptable salt or solvate thereof comprises a heavy chain variable region comprising an amino acid sequence selected from: SEQ ID NOs: 65, 61, 59, or sequence having at least 80%, 85%, 90%, 95% or 99% sequence identity therewith; and/or a light chain variable region comprising an amino acid sequence selected from: SEQ ID NOs: 76, 72, 70, or sequence having at least 80%, 85%, 90%, 95% or 99% sequence identity therewith.

In some embodiments of the present disclosure, the anti-CDH17 antibody or the antigen-binding fragment thereof according to any one of the aforementioned embodiments in the antibody-drug conjugate or the pharmaceutically acceptable salt or solvate thereof comprises: a) the heavy chain variable region having the amino acid sequence as shown in SEQ ID NO: 55, or having the amino acid sequence of at least 80%, 85%, 90%, 95% or 99% sequence identity therewith; and/or the light chain variable region having the amino acid sequence as shown in SEQ ID NO: 66, or having the amino acid sequence of at least 80%, 85%, 90%, 95% or 99% sequence identity therewith; or b) the heavy chain variable region having the amino acid sequence as shown in SEQ ID NO: 56, or having the amino acid sequence of at least 80%, 85%, 90%, 95% or 99% sequence identity therewith; and/or the light chain variable region having the amino acid sequence as shown in SEQ ID NO: 67, or having the amino acid sequence of at least 80%, 85%, 90%, 95% or 99% sequence identity therewith; or c) the heavy chain variable region having the amino acid sequence as shown in SEQ ID NO: 57, or having the amino acid sequence of at least 80%, 85%, 90%, 95% or 99% sequence identity therewith; and/or the light chain variable region having the amino acid sequence as shown in SEQ ID NO: 68, or having the amino acid sequence of at least 80%, 85%, 90%, 95% or 99% sequence identity therewith; or d) the heavy chain variable region having the amino acid sequence as shown in SEQ ID NO: 58, or having the amino acid sequence of at least 80%, 85%, 90%, 95% or 99% sequence identity therewith; and/or the light chain variable region having the amino acid sequence as shown in SEQ ID NO: 69, or having the amino acid sequence of at least 80%, 85%, 90%, 95% or 99% sequence identity therewith; or e) the heavy chain variable region having the amino acid sequence as shown in SEQ ID NO: 59, or having the amino acid sequence of at least 80%, 85%, 90%, 95% or 99% sequence identity therewith; and/or the light chain variable region having the amino acid sequence as shown in SEQ ID NO: 70, or having the amino acid sequence of at least 80%, 85%, 90%, 95% or 99% sequence identity therewith; or f) the heavy chain variable region having the amino acid sequence as shown in SEQ ID NO: 60, or having the amino acid sequence of at least 80%, 85%, 90%, 95% or 99% sequence identity therewith; and/or the light chain variable region having the amino acid sequence as shown in SEQ ID NO: 71, or having the amino acid sequence of at least 80%, 85%, 90%, 95% or 99% sequence identity therewith; or g) the heavy chain variable region having the amino acid sequence as shown in SEQ ID NO: 61, or having the amino acid sequence of at least 80%, 85%, 90%, 95% or 99% sequence identity therewith; and/or the light chain variable region having the amino acid sequence as shown in SEQ ID NO: 72, or having the amino acid sequence of at least 80%, 85%, 90%, 95% or 99% sequence identity therewith; or h) the heavy chain variable region having the amino acid sequence as shown in SEQ ID NO: 62, or having the amino acid sequence of at least 80%, 85%, 90%, 95% or 99% sequence identity therewith; and/or the light chain variable region having the amino acid sequence as shown in SEQ ID NO: 73, or having the amino acid sequence of at least 80%, 85%, 90%, 95% or 99% sequence identity therewith; or i) the heavy chain variable region having the amino acid sequence as shown in SEQ ID NO: 63, or having the amino acid sequence of at least 80%, 85%, 90%, 95% or 99% sequence identity therewith; and/or the light chain variable region having the amino acid sequence as shown in SEQ ID NO: 74, or having the amino acid sequence of at least 80%, 85%, 90%, 95% or 99% sequence identity therewith; or j) the heavy chain variable region having the amino acid sequence as shown in SEQ ID NO: 64, or having the amino acid sequence of at least 80%, 85%, 90%, 95% or 99% sequence identity therewith; and/or the light chain variable region having the amino acid sequence as shown in SEQ ID NO: 75, or having the amino acid sequence of at least 80%, 85%, 90%, 95% or 99% sequence identity therewith; or k) the heavy chain variable region having the amino acid sequence as shown in SEQ ID NO: 65, or having the amino acid sequence of at least 80%, 85%, 90%, 95% or 99% sequence identity therewith; and/or the light chain variable region having the amino acid sequence as shown in SEQ ID NO: 76, or having the amino acid sequence of at least 80%, 85%, 90%, 95% or 99% sequence identity therewith.

In some embodiments of the present disclosure, the anti-CDH17 antibody or the antigen-binding fragment thereof according to any one of the aforementioned embodiments in the antibody-drug conjugate or the pharmaceutically acceptable salt or solvate thereof comprise: a) the heavy chain variable region having the amino acid sequence as shown in SEQ ID NO: 55; and/or the light chain variable region having the amino acid sequence as shown in SEQ ID NO: 66; or b) the heavy chain variable region having the amino acid sequence as shown in SEQ ID NO: 56; and/or the light chain variable region having the amino acid sequence as shown in SEQ ID NO: 67; or c) the heavy chain variable region having the amino acid sequence as shown in SEQ ID NO: 57; and/or the light chain variable region having the amino acid sequence as shown in SEQ ID NO: 68; or d) the heavy chain variable region having the amino acid sequence as shown in SEQ ID NO: 58; and/or the light chain variable region having the amino acid sequence as shown in SEQ ID NO: 69; or e) the heavy chain variable region having the amino acid sequence as shown in SEQ ID NO: 59; and/or the light chain variable region having the amino acid sequence as shown in SEQ ID NO: 70; or f) the heavy chain variable region having the amino acid sequence as shown in SEQ ID NO: 60; and/or the light chain variable region having the amino acid sequence as shown in SEQ ID NO: 71; or g) the heavy chain variable region having the amino acid sequence as shown in SEQ ID NO: 61; and/or the light chain variable region having the amino acid sequence as shown in SEQ ID NO: 72; or h) the heavy chain variable region having the amino acid sequence as shown in SEQ ID NO: 62; and/or the light chain variable region having the amino acid sequence as shown in SEQ ID NO: 73; or i) the heavy chain variable region having the amino acid sequence as shown in SEQ ID NO: 63; and/or the light chain variable region having the amino acid sequence as shown in SEQ ID NO: 74; or j) the heavy chain variable region having the amino acid sequence as shown in SEQ ID NO: 64; and/or the light chain variable region having the amino acid sequence as shown in SEQ ID NO: 75; or k) the heavy chain variable region having the amino acid sequence as shown in SEQ ID NO: 65; and/or the light chain variable region having the amino acid sequence as shown in SEQ ID NO: 76.

In some embodiments of the present disclosure, the anti-CDH17 antibody or the antigen-binding fragment thereof according to any one of the aforementioned embodiments in the antibody-drug conjugate or the pharmaceutically acceptable salt or solvate thereof comprises: a) the heavy chain variable region having the amino acid sequence as shown in SEQ ID NO: 55; and the light chain variable region having the amino acid sequence as shown in SEQ ID NO: 66; or b) the heavy chain variable region having the amino acid sequence as shown in SEQ ID NO: 56; and the light chain variable region having the amino acid sequence as shown in SEQ ID NO: 67; or c) the heavy chain variable region having the amino acid sequence as shown in SEQ ID NO: 57; and the light chain variable region having the amino acid sequence as shown in SEQ ID NO: 68; or d) the heavy chain variable region having the amino acid sequence as shown in SEQ ID NO: 58; and the light chain variable region having the amino acid sequence as shown in SEQ ID NO: 69; or e) the heavy chain variable region having the amino acid sequence as shown in SEQ ID NO: 59; and the light chain variable region having the amino acid sequence as shown in SEQ ID NO: 70; or f) the heavy chain variable region having the amino acid sequence as shown in SEQ ID NO: 60; and the light chain variable region having the amino acid sequence as shown in SEQ ID NO: 71; or g) the heavy chain variable region having the amino acid sequence as shown in SEQ ID NO: 61; and the light chain variable region having the amino acid sequence as shown in SEQ ID NO: 72; or h) the heavy chain variable region having the amino acid sequence as shown in SEQ ID NO: 62; and the light chain variable region having the amino acid sequence as shown in SEQ ID NO: 73; or i) the heavy chain variable region having the amino acid sequence as shown in SEQ ID NO: 63; and the light chain variable region having the amino acid sequence as shown in SEQ ID NO: 74; or j) the heavy chain variable region having the amino acid sequence as shown in SEQ ID NO: 64; and the light chain variable region having the amino acid sequence as shown in SEQ ID NO: 75; or k) the heavy chain variable region having the amino acid sequence as shown in SEQ ID NO: 65; and the light chain variable region having the amino acid sequence as shown in SEQ ID NO: 76.

In some embodiments of the present disclosure, the anti-CDH17 antibody or the antigen-binding fragment thereof according to any one of the aforementioned embodiments in the antibody-drug conjugate or the pharmaceutically acceptable salt or solvate thereof comprises human antibody constant regions; preferably, the heavy chain constant region of the human antibody constant regions is selected from constant regions of human IgG1, IgG2, IgG3 and IgG4 and conventional variants thereof, and the light chain constant region of the human antibody constant regions is selected from κ and λ chain constant regions of human antibody and conventional variants thereof; more preferably, the full-length antibody comprises a human antibody heavy chain constant region of SEQ ID NO: 99 or having the amino acid sequence of at least 80%, 85%, 90%, 95% or 99% sequence identity therewith, a human light chain constant region of SEQ ID NO: 100 or having the amino acid sequence of at least 80%, 85%, 90%, 95% or 99% sequence identity therewith; further preferably the full-length antibody comprises a human antibody heavy chain constant region of SEQ ID NO: 99 and a human light chain constant region of SEQ ID NO: 100.

In some embodiments of the present disclosure, the anti-CDH17 antibody or the antigen-binding fragment thereof according to any one of the aforementioned embodiments in the antibody-drug conjugate or the pharmaceutically acceptable salt or solvate thereof comprises a heavy chain having the amino acid sequence as shown in SEQ ID NO: 97 or having the amino acid sequence of at least 80%, 85%, 90%, 95% or 99% sequence identity therewith, and a light chain having the amino acid sequence as shown in SEQ ID NO: 98 or having the amino acid sequence of at least 80%, 85%, 90%, 95% or 99% sequence identity therewith; or
a heavy chain having the amino acid sequence as shown in SEQ ID NO: 89 or having the amino acid sequence of at least 80%, 85%, 90%, 95% or 99% sequence identity therewith, and a light chain having the amino acid sequence as shown in SEQ ID NO: 90 or having the amino acid sequence of at least 80%, 85%, 90%, 95% or 99% sequence identity therewith; or
a heavy chain having the amino acid sequence as shown in SEQ ID NO: 85 or having the amino acid sequence of at least 80%, 85%, 90%, 95% or 99% sequence identity therewith, and a light chain having the amino acid sequence as shown in SEQ ID NO: 86 or having the amino acid sequence of at least 80%, 85%, 90%, 95% or 99% sequence identity therewith.

In some embodiments of the present disclosure, the anti-CDH17 antibody or the antigen-binding fragment according to any one of the aforementioned embodiments in the antibody-drug conjugate or the pharmaceutically acceptable salt or solvate thereof comprises: a) a heavy chain having the amino acid sequence as shown in SEQ ID NO:77 and a light chain having the amino acid sequence as shown in SEQ ID NO:78; or b) a heavy chain having the amino acid sequence as shown in SEQ ID NO:79 and a light chain having the amino acid sequence as shown in SEQ ID NO:80; or c) a heavy chain having the amino acid sequence as shown in SEQ ID NO:81 and a light chain having the amino acid sequence as shown in SEQ ID NO:82; or d) a heavy chain having the amino acid sequence as shown in SEQ ID NO:83 and a light chain having the amino acid sequence as shown in SEQ ID NO:84; or e) a heavy chain having the amino acid sequence as shown in SEQ ID NO:85 and a light chain having the amino acid sequence as shown in SEQ ID NO:86; or f) a heavy chain having the amino acid sequence as shown in SEQ ID NO:87 and a light chain having the amino acid sequence as shown in SEQ ID NO:88; or g) a heavy chain having the amino acid sequence as shown in SEQ ID NO:89 and a light chain having the amino acid sequence as shown in SEQ ID NO:90; or h) a heavy chain having the amino acid sequence as shown in SEQ ID NO:91 and a light chain having the amino acid sequence as shown in SEQ ID NO:92; or i) a heavy chain having the amino acid sequence as shown in SEQ ID NO:93 and a light chain having the amino acid sequence as shown in SEQ ID NO:94; or j) a heavy chain having the amino acid sequence as shown in SEQ ID NO:95 and a light chain having the amino acid sequence as shown in SEQ ID NO:96; or k) a heavy chain having the amino acid sequence as shown in SEQ ID NO:97 and a light chain having the amino acid sequence as shown in SEQ ID NO:98.

In some embodiments of the present disclosure, the anti-CDH17 antibody or the antigen-binding fragment in the antibody-drug conjugate or the pharmaceutically acceptable salt or solvate thereof, the anti-CDH17 antigen-binding fragment is selected from the group consisting of Fab, Fab′, F(ab′)2, variable fragment (Fv), single chain variable fragment (scFv), dimerized domain V (diabody), disulfide stabilized Fv (dsFv) and CDR-containing peptides.

In some embodiments of the present disclosure, the toxin drug in the antibody-drug conjugate or the pharmaceutically acceptable salt or solvate thereof according to any one of the aforementioned embodiments is selected from the group consisting of tubulin inhibitors, topoisomerase inhibitors, DNA intercalators, and RNA polymerase inhibitors or pharmaceutically acceptable salt, ester or analog thereof, preferably the toxin drug is selected from the group consisting of auristatin analogues, camptothecin derivatives, maytansine analogues; more preferably the toxin drug is selected from the group consisting of MMAE, MMAF, Exatecan, MMAD, DM1, DM4, eribulin, pyrrolobenzodiazepine(PBD), DGN-549-C, SN-38, irinotecan, topotecan, belotecan, rubitecan, doxorubicin, PNU-159682, duocarmycin, daunorubicin, mitoxantrone, podophyllotoxin, etoposide, α-amanitin, or a pharmaceutically acceptable salt, ester or analog thereof.

In some embodiments of the present disclosure, the antibody-drug conjugate or the pharmaceutically acceptable salt or solvate thereof according to any one of the aforementioned embodiments is an antibody drug conjugate of general formula (A):

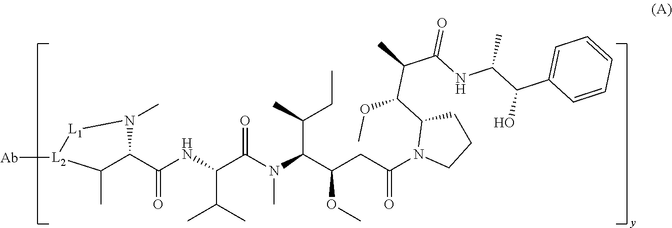

wherein: L₁ and L₂ are linking units; y is a number selected from 1 to 10, preferably a number selected from 2 to 8, and most preferably 2, 4, 5, 6, 7, 8; Ab is the anti-CDH17 antibody or antigen-binding fragment thereof mention above.

In some embodiments of the present disclosure, the antibody-drug conjugate or the pharmaceutically acceptable salt or solvate thereof according to any one of the aforementioned embodiments is an antibody drug conjugate of general formula (I):

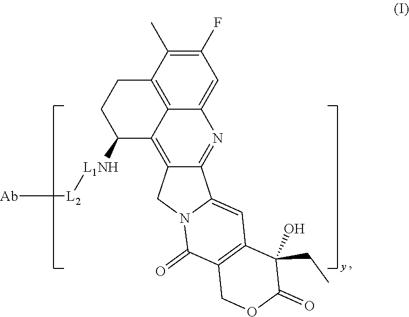

wherein: L₁ and L₂ are linking units; y is a number selected from 1 to 10, preferably a number selected from 2 to 8, and most preferably 2, 4, 6, 8; Ab is the anti-CDH17 antibody or antigen-binding fragment thereof mention above.

In some embodiments of the present disclosure, in the antibody-drug conjugate or the pharmaceutically acceptable salt or solvate thereof according to any one of the aforementioned embodiments wherein L₁ is selected from the group consisting of:

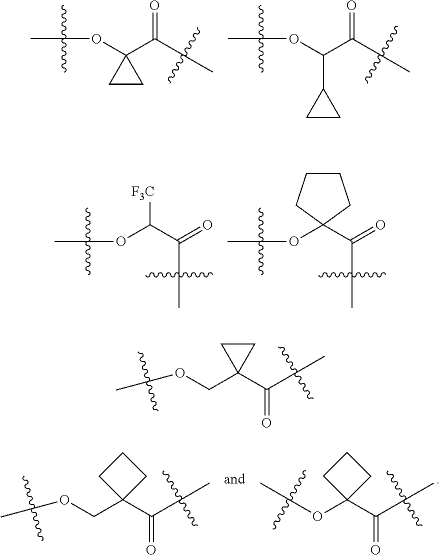

In some embodiments of the present disclosure, in the antibody-drug conjugate or the pharmaceutically acceptable salt or solvate thereof according to any one of the aforementioned embodiments wherein L₂ is -L^{a}-L^{b}-L^{c}-L^{d}-, wherein
the L^{a} is as shown in general formula (II-a):

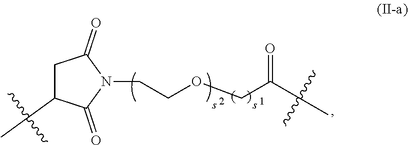

wherein: s¹ and s² are each independently an integer selected from 0-8, Preferably, s¹ and s² are independently selected from 1, 2, 3, 4, 5 or 6; Or, s is an integer from 1-8, s² is 0, Preferably, s¹ is selected from 4, 5, 6, 7 or 8, and s² is 0; Or, s² is selected from an integer from 2 to 8, s¹ is 2, Preferably, s² is selected from 2, 3, 4, 5 or 6, and s¹ is 2; the L^{b} is a chemical bond; the L^{c} is a tetrapeptide residue; preferably, L^{c} is a tetrapeptide residue of glycine-glycine-phenylalanine-glycine (GGFG); the L^{d} is —NR₁(CR₂R₃)s³-, wherein R₁, R₂ and R₃ are identical or different and are each independently hydrogen or alkyl, and s³ is 1 or 2; wherein the L^{a} terminus is connected to Ab, and the L^{d} terminus is connected to L₁.

In some embodiments of the present disclosure, the antibody-drug conjugate of general formula (I), or a pharmaceutically acceptable salt or solvate thereof,

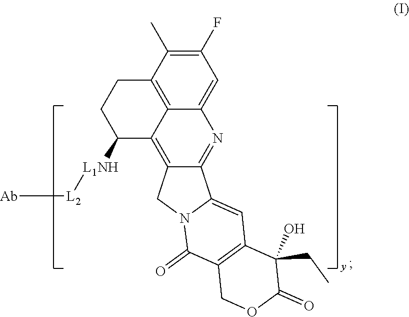

wherein:
L₁ and L₂ are linking units; wherein the L₂ is -La-Lb-Lc-Ld-,
the La is as shown in general formula (II-b):

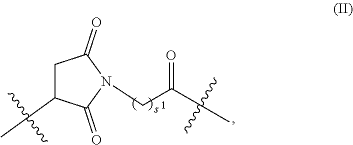

s1 is selected from 4, 5, 6, 7 or 8;
the L^{b} is a chemical bond;
the Lc is a tetrapeptide residue; preferably, Lc is a tetrapeptide residue of glycine-glycine-phenylalanine-glycine (GGFG);
the Ld is —NR1(CR2R3)s3-, wherein R1, R2 and R3 are identical or different and are each independently hydrogen or alkyl, and s3 is 1 or 2; wherein the La terminus is connected to Ab, and the Ld terminus is connected to L1;
y is a number selected from 1 to 10, preferably a number selected from 2 to 8, and most preferably 2, 4, 6, 8;
Ab is the anti-CDH17 antibody or antigen-binding fragment thereof, wherein the anti-CDH17 antibody or the antigen-binding fragment thereof comprises a heavy chain variable region and a light chain variable region of the antibody, wherein:
a heavy chain variable region comprises HCDR1, HCDR2 and HCDR3 having the amino acid sequences as shown in SEQ ID NO: 10, SEQ ID NO:21 and SEQ ID NO: 31, respectively; and a light chain variable region comprises LCDR1, LCDR2 and LCDR3 having the amino acid sequences as shown in SEQ ID NO: 42, SEQ ID NO: 47 and SEQ ID NO: 54, respectively; or
a heavy chain variable region comprises HCDR1, HCDR2 and HCDR3 having the amino acid sequences as shown in SEQ ID NO: 07, SEQ ID NO: 17 and SEQ ID NO: 28, respectively; and a light chain variable region comprises LCDR1, LCDR2 and LCDR3 having the amino acid sequences as shown in SEQ ID NO: 38, SEQ ID NO: 45 and SEQ ID NO: 53, respectively; or
a heavy chain variable region comprises HCDR1, HCDR2 and HCDR3 having the amino acid sequences as shown in SEQ ID NO: 05, SEQ ID NO: 15 and SEQ ID NO: 26, respectively; and a light chain variable region comprises LCDR1, LCDR2 and LCDR3 having the amino acid sequences as shown in SEQ ID NO: 36, SEQ ID NO: 43 and SEQ ID NO: 51, respectively.

In some embodiments of the present disclosure, the anti-CDH17 antibody or antigen-binding fragment thereof is a monoclonal antibody or antigen-binding fragment thereof, a polyclonal antibody or antigen-binding fragment thereof, a multi-specific antibody or antigen-binding fragment thereof, a murine antibody or antigen-binding fragment thereof, a chimeric antibody or antigen-binding fragment thereof, a humanized antibody or antigen-binding fragment thereof, a recombinant antibody or antigen-binding fragment thereof, a human antibody or antigen-binding fragment thereof; preferably, the multi-specific antibody is a bispecific antibody, a tri-specific antibody or a tetra-specific antibody.

In some embodiments of the present disclosure, the anti-CDH17 antibody or antigen-binding fragment thereof comprises: a heavy chain variable region comprising an amino acid sequence selected from: SEQ ID NOs: 65, 61, 59, or sequence having at least 80%, 85%, 90%, 95% or 99% sequence identity therewith; and/or a light chain variable region comprising an amino acid sequence selected from: SEQ ID NOs: 76, 72, 70, or sequence having at least 80%, 85%, 90%, 95% or 99% sequence identity therewith.

In some embodiments of the present disclosure, the anti-CDH17 antibody or antigen-binding fragment thereof comprising: the heavy chain variable region having the amino acid sequence as shown in SEQ ID NO: 65, or having the amino acid sequence of at least 80%, 85%, 90%, 95% or 99% sequence identity therewith; and/or the light chain variable region having the amino acid sequence as shown in SEQ ID NO: 76, or having the amino acid sequence of at least 80%, 85%, 90%, 95% or 99% sequence identity therewith; or the heavy chain variable region having the amino acid sequence as shown in SEQ ID NO: 61, or having the amino acid sequence of at least 80%, 85%, 90%, 95% or 99% sequence identity therewith; and/or the light chain variable region having the amino acid sequence as shown in SEQ ID NO: 72, or having the amino acid sequence of at least 80%, 85%, 90%, 95% or 99% sequence identity therewith; or the heavy chain variable region having the amino acid sequence as shown in SEQ ID NO: 59, or having the amino acid sequence of at least 80%, 85%, 90%, 95% or 99% sequence identity therewith; and/or the light chain variable region having the amino acid sequence as shown in SEQ ID NO: 70, or having the amino acid sequence of at least 80%, 85%, 90%, 95% or 99% sequence identity therewith;

In some embodiments of the present disclosure, the anti-CDH17 antibody or antigen-binding fragment thereof comprising: the heavy chain variable region having the amino acid sequence as shown in SEQ ID NO: 65; and/or the light chain variable region having the amino acid sequence as shown in SEQ ID NO: 76; or the heavy chain variable region having the amino acid sequence as shown in SEQ ID NO: 61; and/or the light chain variable region having the amino acid sequence as shown in SEQ ID NO: 72; or the heavy chain variable region having the amino acid sequence as shown in SEQ ID NO: 59; and/or the light chain variable region having the amino acid sequence as shown in SEQ ID NO: 70.

In some embodiments of the present disclosure, the anti-CDH17 antibody or antigen-binding fragment thereof comprising: the heavy chain variable region having the amino acid sequence as shown in SEQ ID NO: 65 and the light chain variable region having the amino acid sequence as shown in SEQ ID NO: 76; or the heavy chain variable region having the amino acid sequence as shown in SEQ ID NO: 61 and the light chain variable region having the amino acid sequence as shown in SEQ ID NO: 72; or the heavy chain variable region having the amino acid sequence as shown in SEQ ID NO: 59 and the light chain variable region having the amino acid sequence as shown in SEQ ID NO: 70.

In some embodiments of the present disclosure, the anti-CDH17 antibody or antigen-binding fragment thereof further comprising human antibody constant regions; preferably, the heavy chain constant region of the human antibody constant regions is selected from constant regions of human IgG1, IgG2, IgG3 and IgG4 and conventional variants thereof, and the light chain constant region of the human antibody constant regions is selected from κ and λ chain constant regions of human antibody and conventional variants thereof; more preferably, the full-length antibody comprises a human antibody heavy chain constant region of SEQ ID NO: 99 or having the amino acid sequence of at least 80%, 85%, 90%, 95% or 99% sequence identity therewith, a human light chain constant region of SEQ ID NO: 100 or having the amino acid sequence of at least 80%, 85%, 90%, 95% or 99% sequence identity therewith; further preferably, the full-length antibody comprises a human antibody heavy chain constant region of SEQ ID NO: 99 and a human light chain constant region of SEQ ID NO: 100.

In some embodiments of the present disclosure, the anti-CDH17 antibody or antigen-binding fragment thereof comprising: a heavy chain having the amino acid sequence as shown in SEQ ID NO: 97 or having the amino acid sequence of at least 80%, 85%, 90%, 95% or 99% sequence identity therewith, and a light chain having the amino acid sequence as shown in SEQ ID NO: 98 or having the amino acid sequence of at least 80%, 85%, 90%, 95% or 99% sequence identity therewith; or a heavy chain having the amino acid sequence as shown in SEQ ID NO: 89 or having the amino acid sequence of at least 80%, 85%, 90%, 95% or 99% sequence identity therewith, and a light chain having the amino acid sequence as shown in SEQ ID NO: 90 or having the amino acid sequence of at least 80%, 85%, 90%, 95% or 99% sequence identity therewith; or a heavy chain having the amino acid sequence as shown in SEQ ID NO: 85 or having the amino acid sequence of at least 80%, 85%, 90%, 95% or 99% sequence identity therewith, and a light chain having the amino acid sequence as shown in SEQ ID NO: 86 or having the amino acid sequence of at least 80%, 85%, 90%, 95% or 99% sequence identity therewith.

In some embodiments of the present disclosure, the anti-CDH17 antibody or antigen-binding fragment thereof comprising: a heavy chain having the amino acid sequence as shown in SEQ ID NO: 97 and a light chain having the amino acid sequence as shown in SEQ ID NO: 98; or a heavy chain having the amino acid sequence as shown in SEQ ID NO: 89 and a light chain having the amino acid sequence as shown in SEQ ID NO: 90; or a heavy chain having the amino acid sequence as shown in SEQ ID NO: 85 and a light chain having the amino acid sequence as shown in SEQ ID NO: 86.

In some embodiments of the present disclosure, the anti-CDH17 antigen-binding fragment is selected from the group consisting of Fab, Fab′, F(ab′)2, variable fragment (Fv), single chain variable fragment (scFv), dimerized domain V (diabody), disulfide stabilized Fv (dsFv) and CDR-containing peptides.

In some embodiments of the present disclosure, the antibody-drug conjugate or the pharmaceutically acceptable salt or solvate thereof according to any one of the aforementioned embodiments is as the following structure:

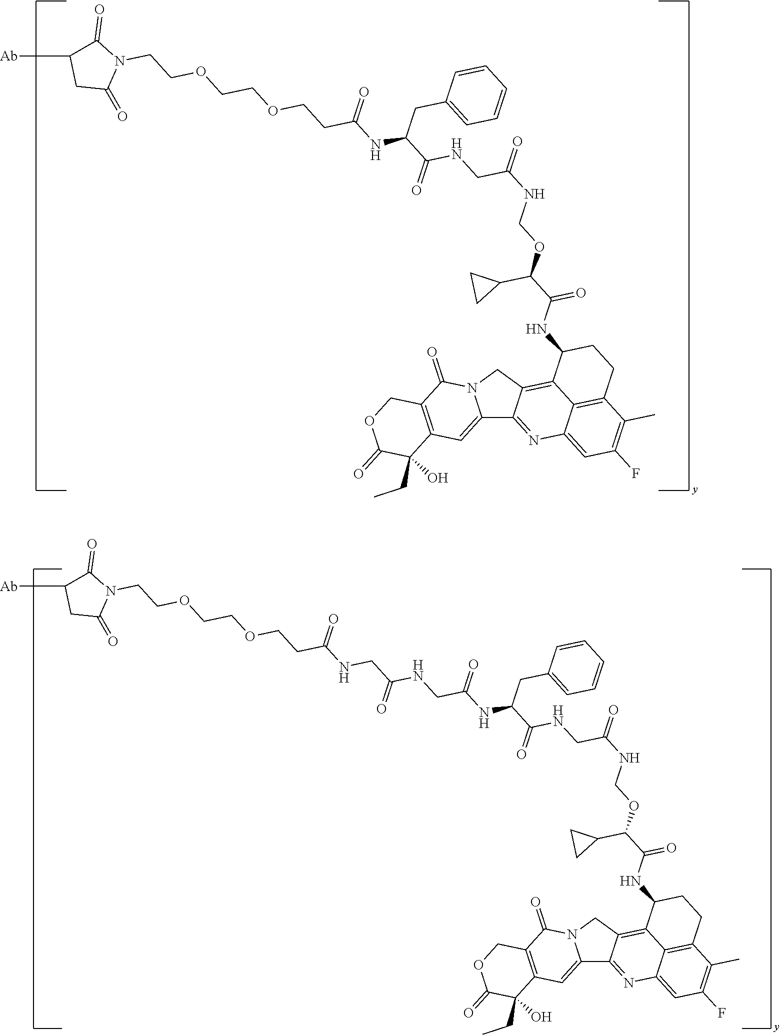

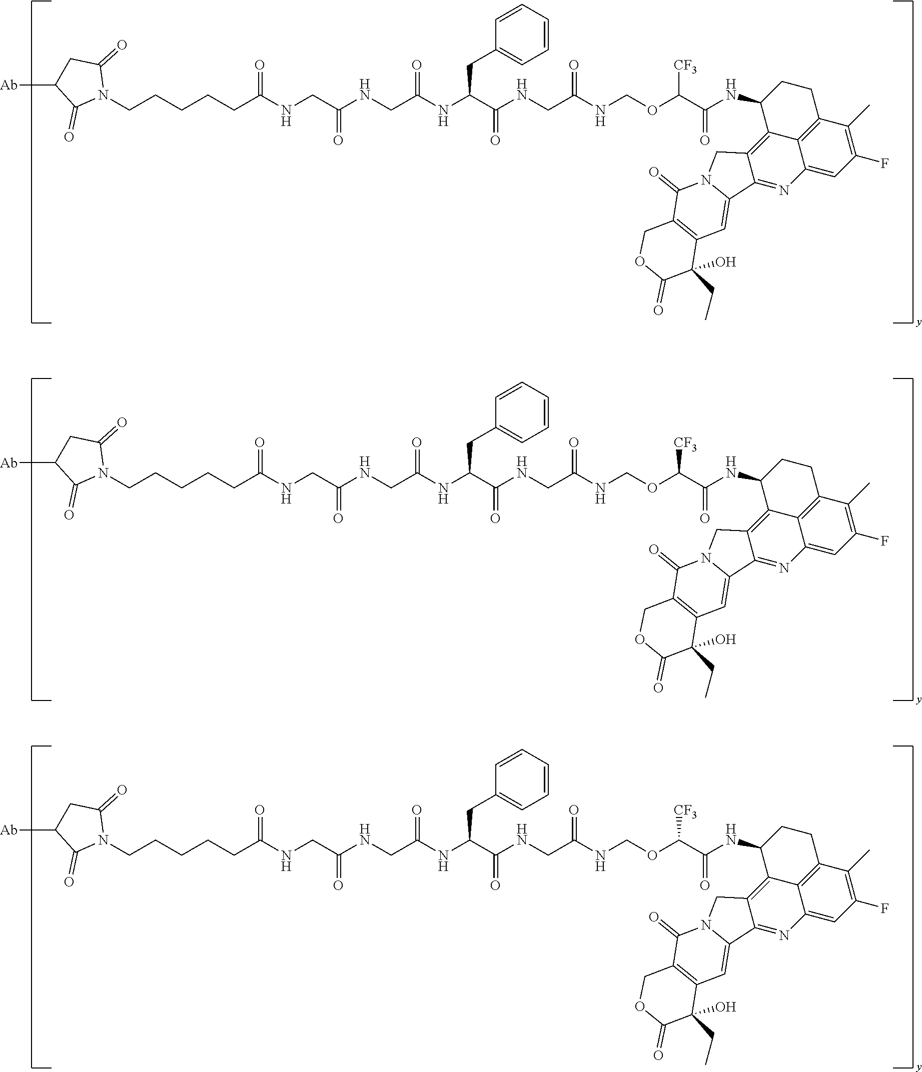

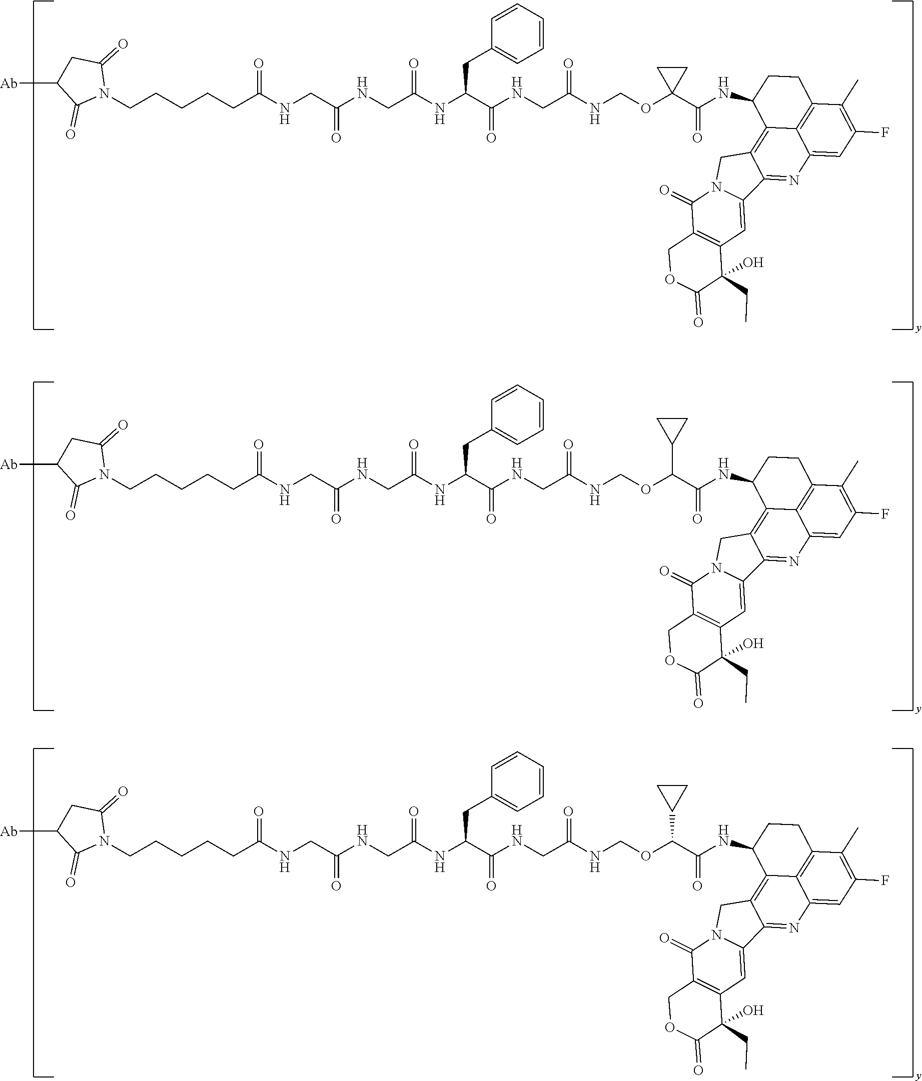

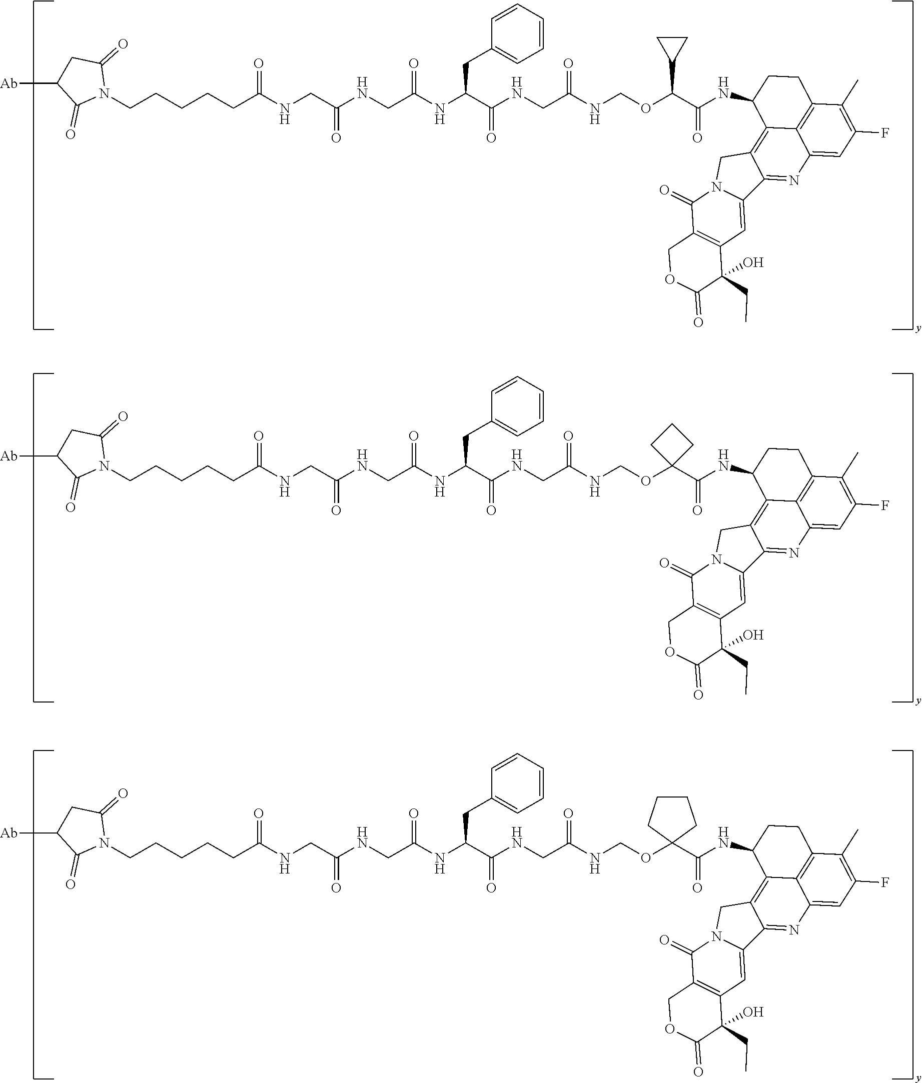

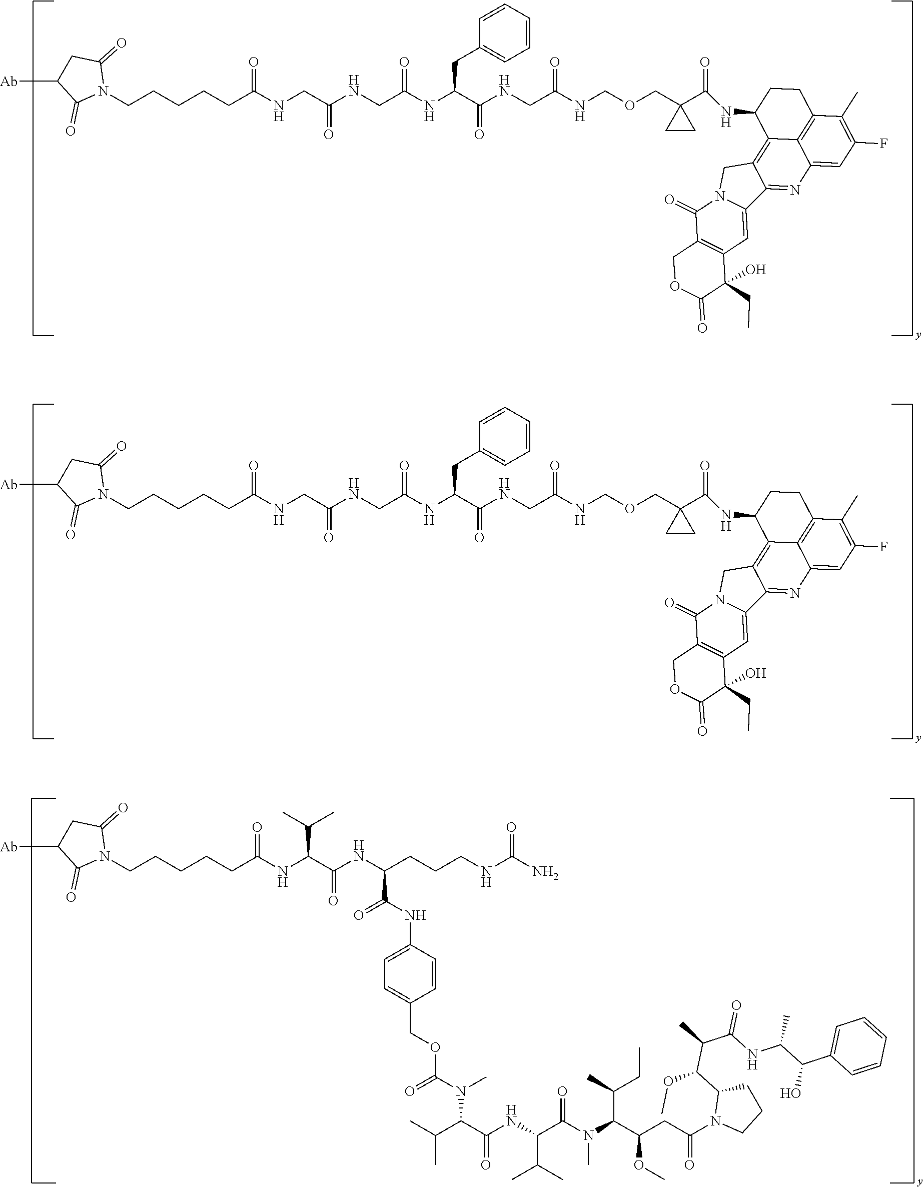

y is a number selected from 1 to 10, preferably a number selected from 2 to 8, and most preferably 2, 4, 5, 6, 7, 8; Ab is the anti-CDH17 antibody or antigen-binding fragment thereof mention above.

In some embodiments of the present disclosure, the antibody-drug conjugate or the pharmaceutically acceptable salt or solvate thereof according to any one of the aforementioned embodiments is selected from the group consisting of the following compounds:

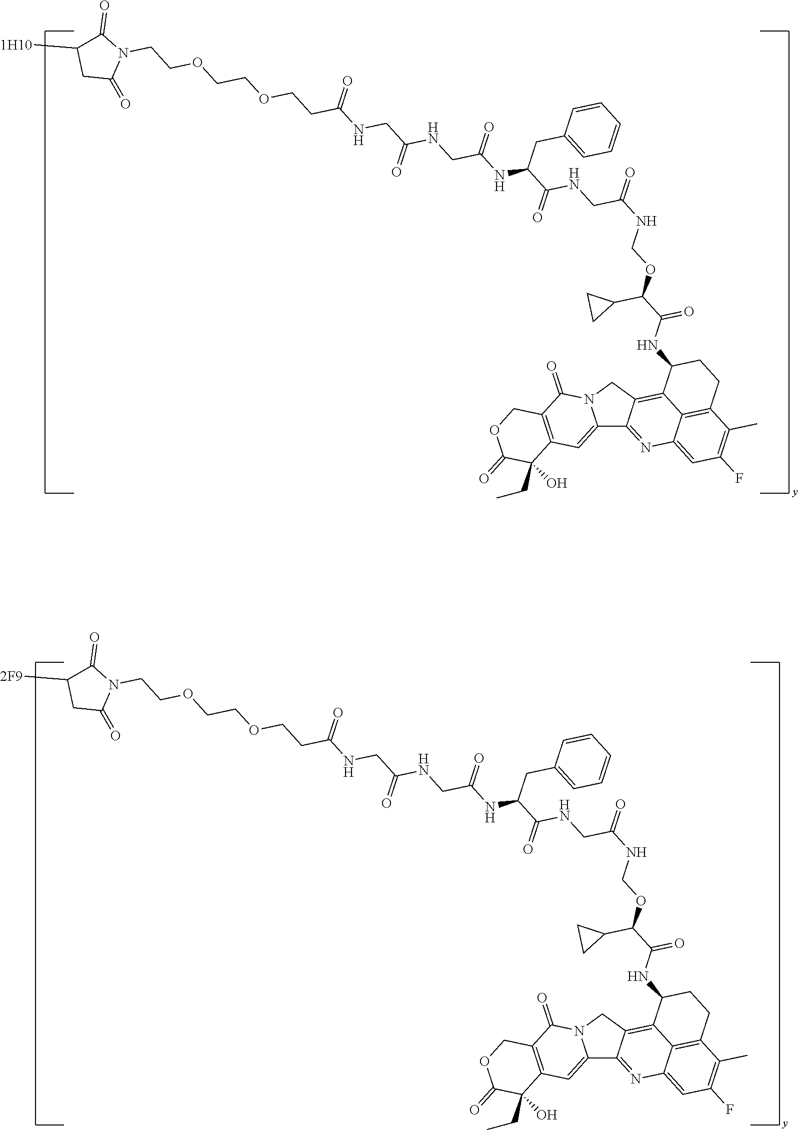

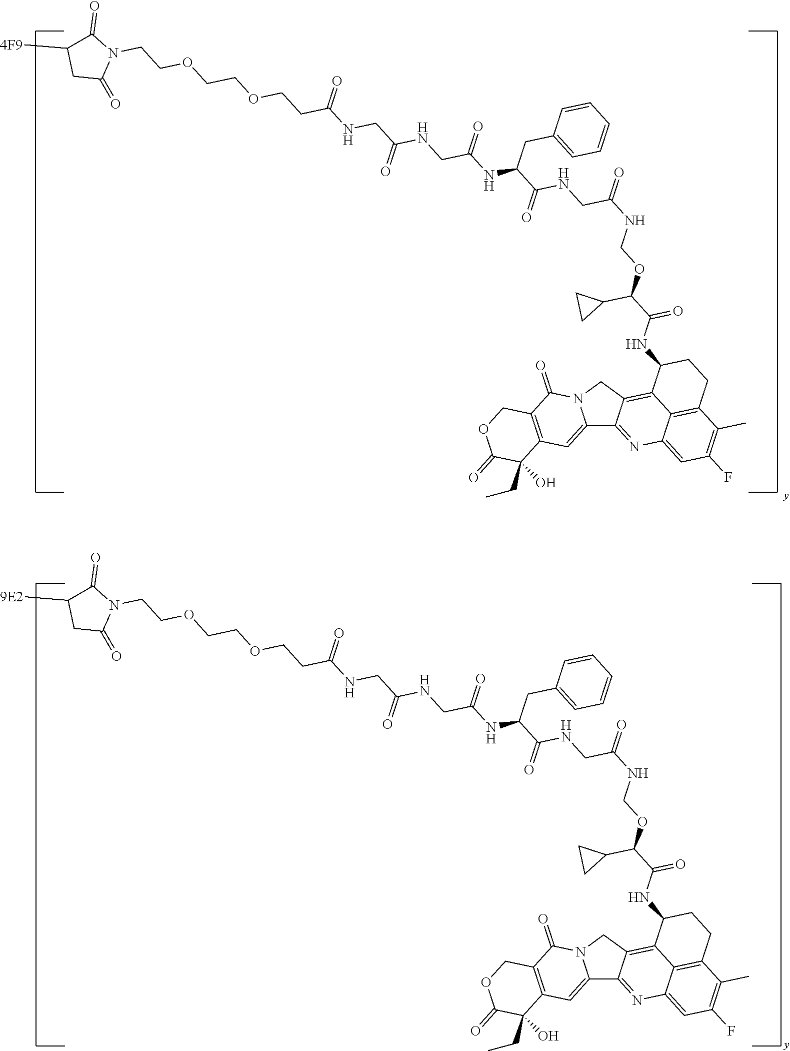

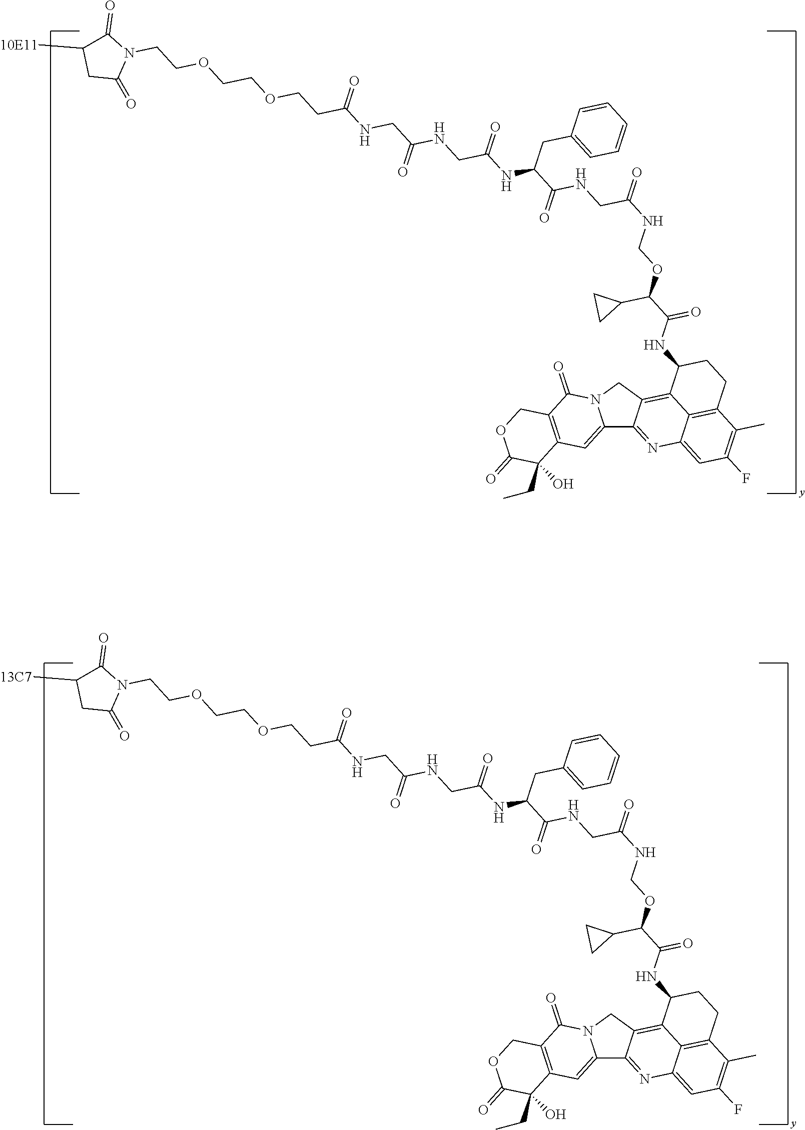

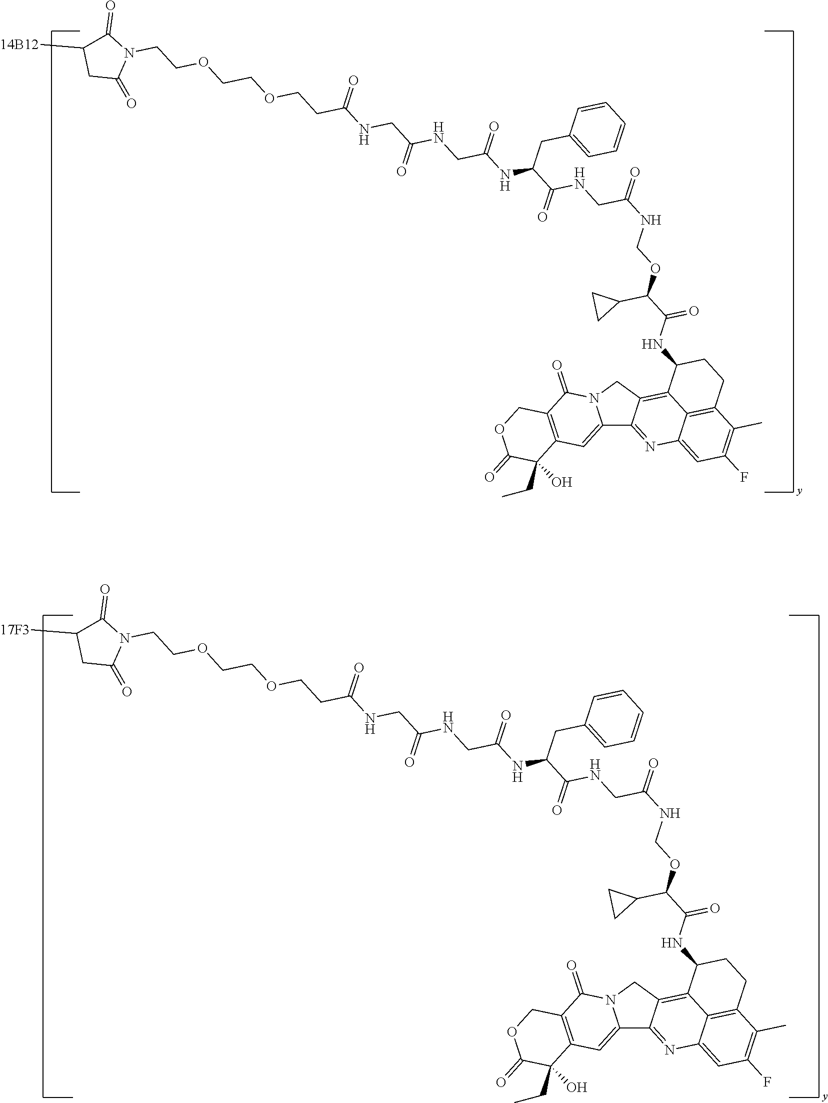

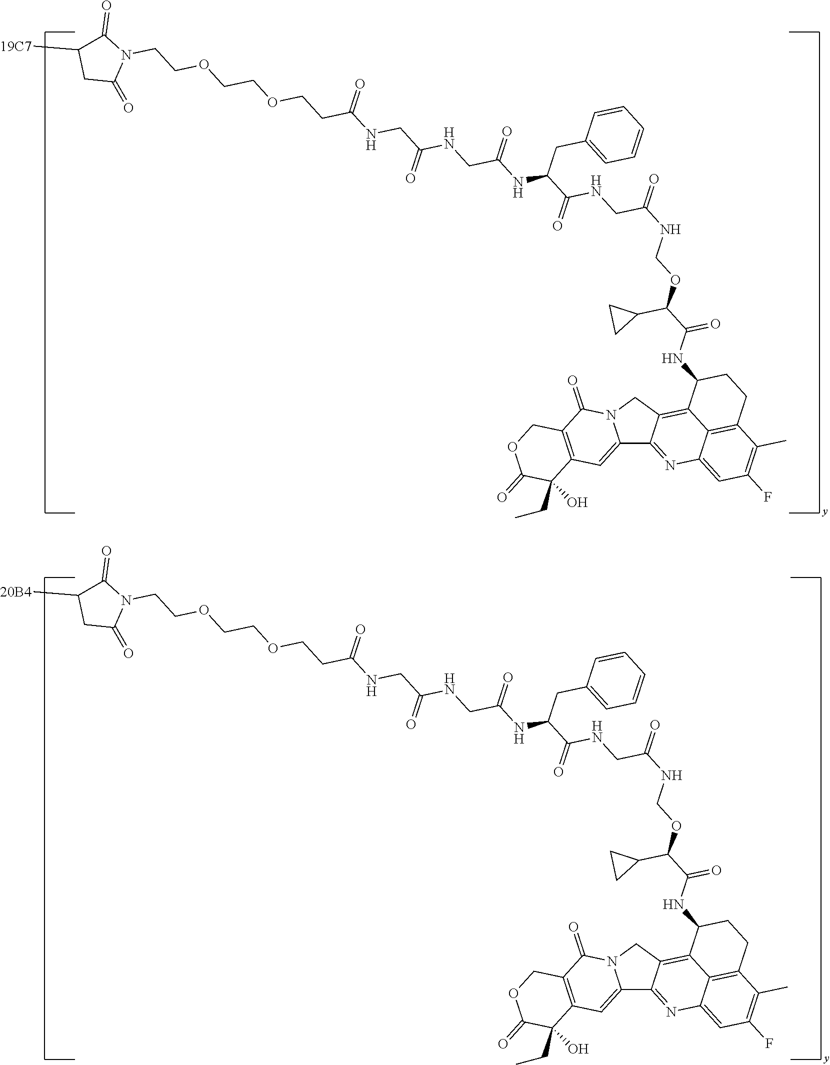

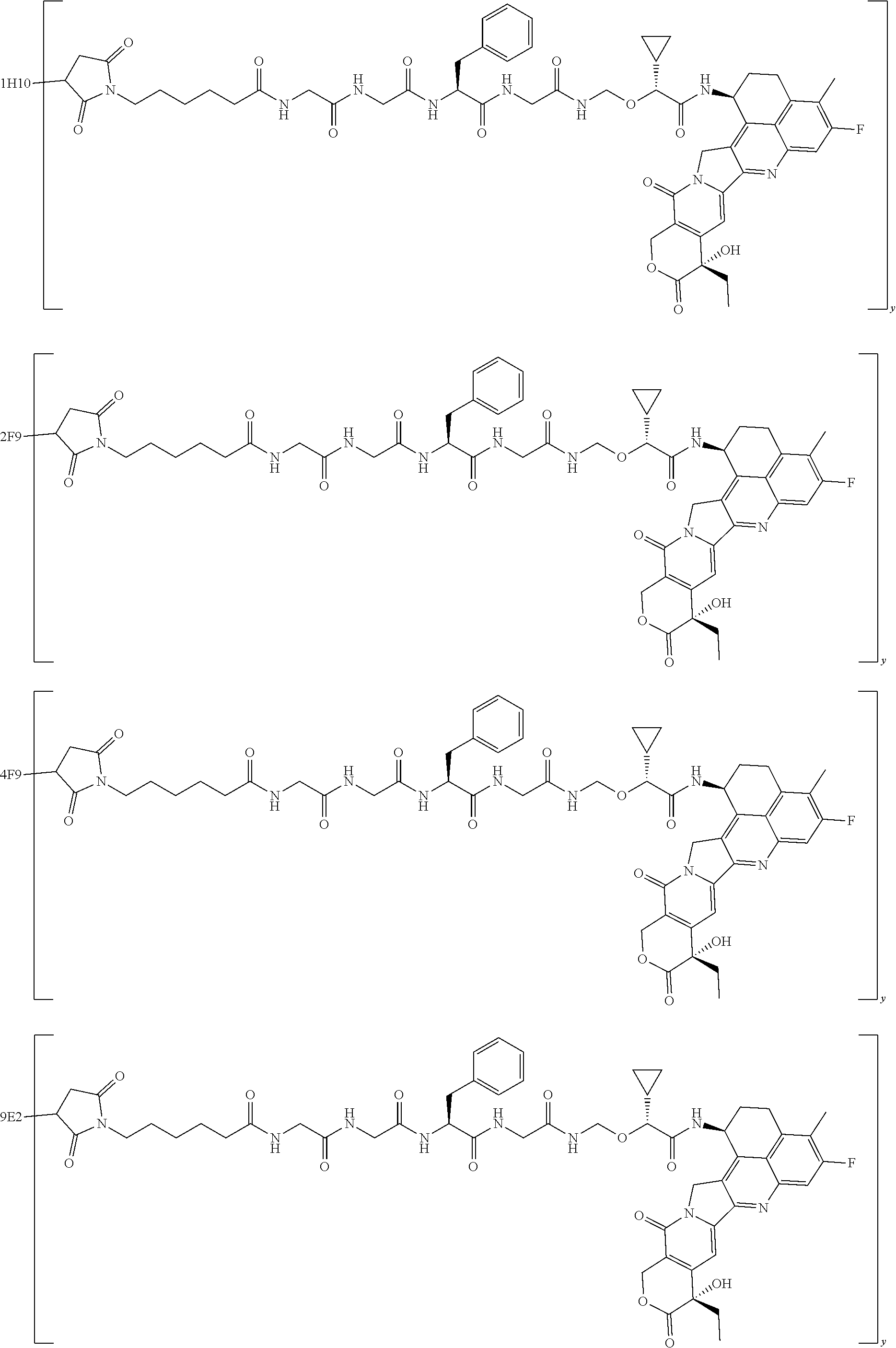

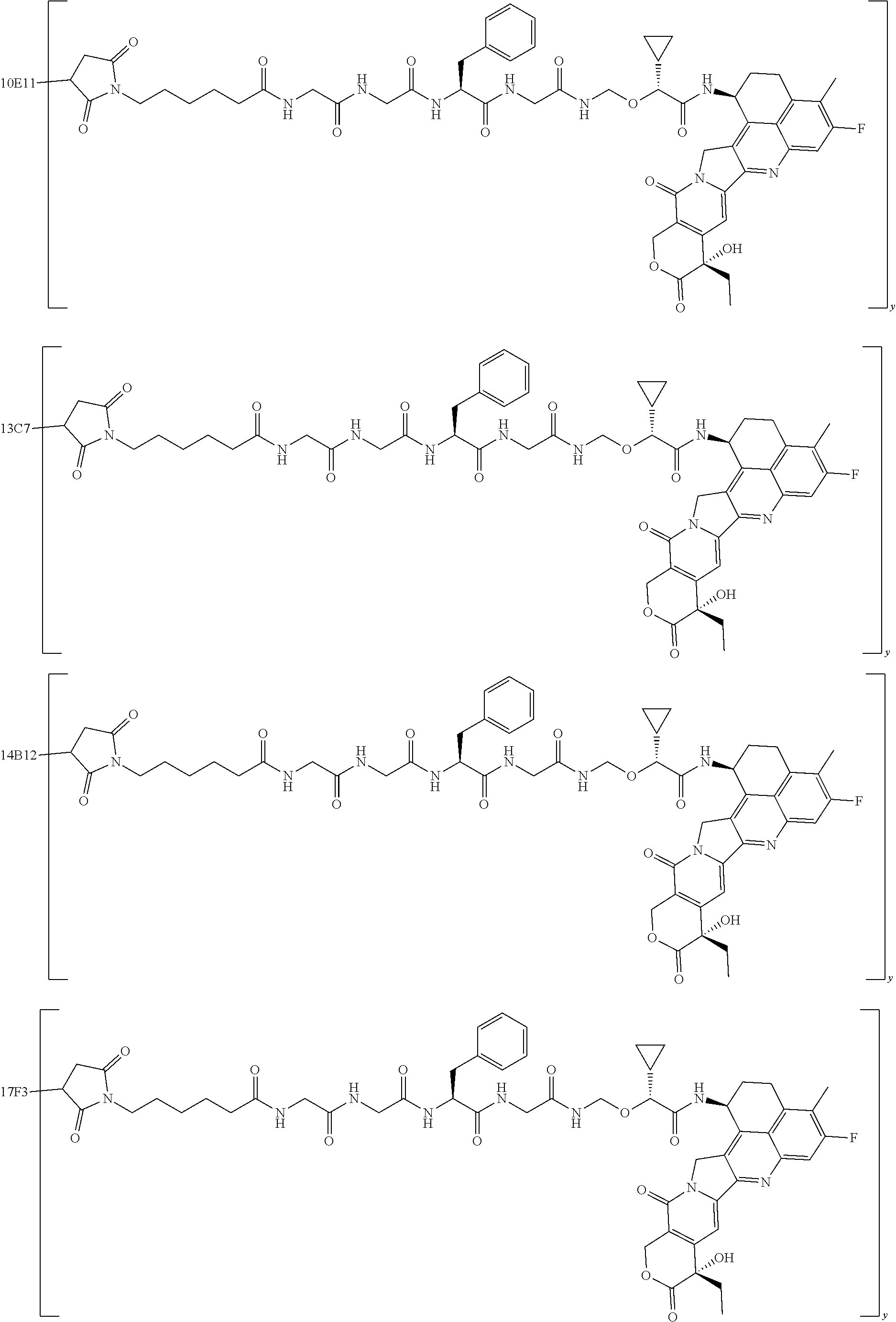

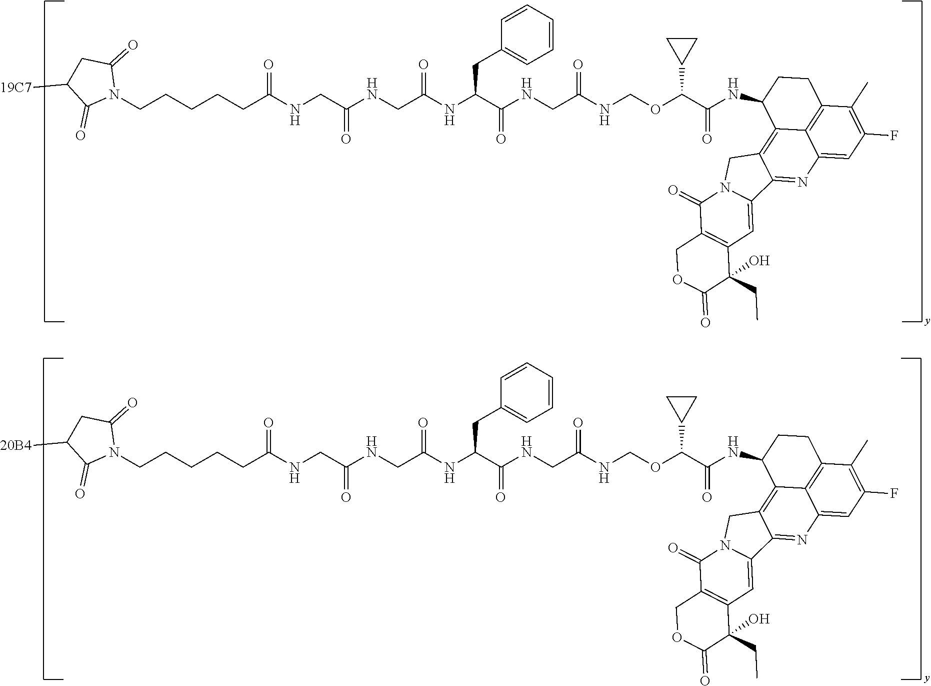

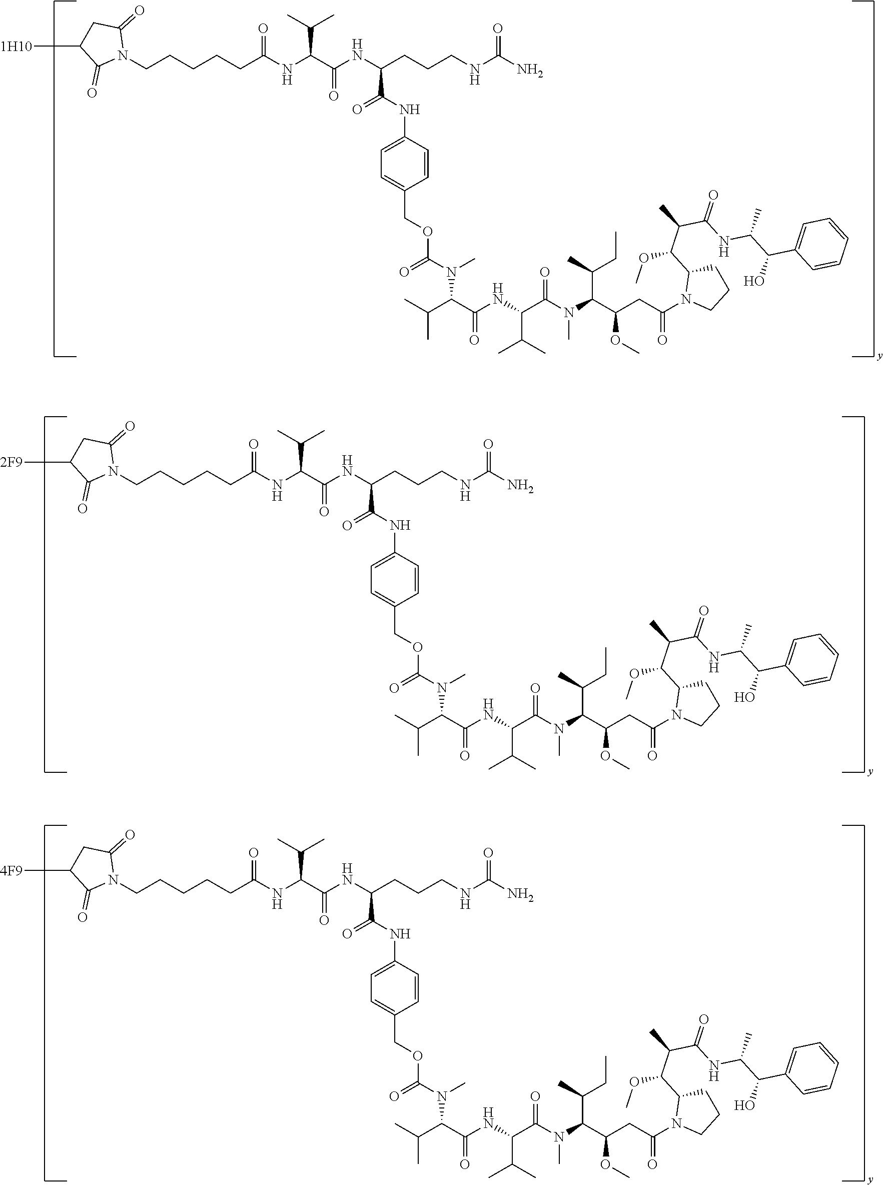

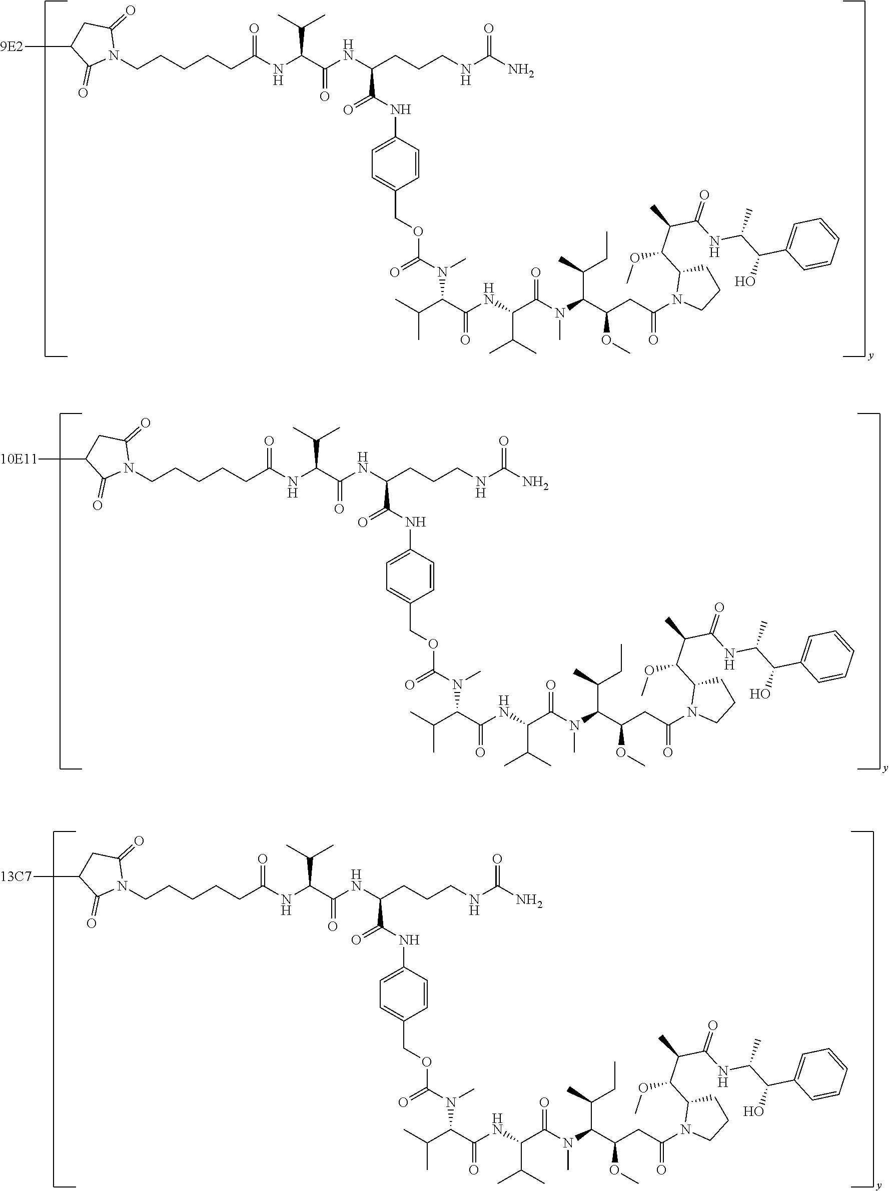

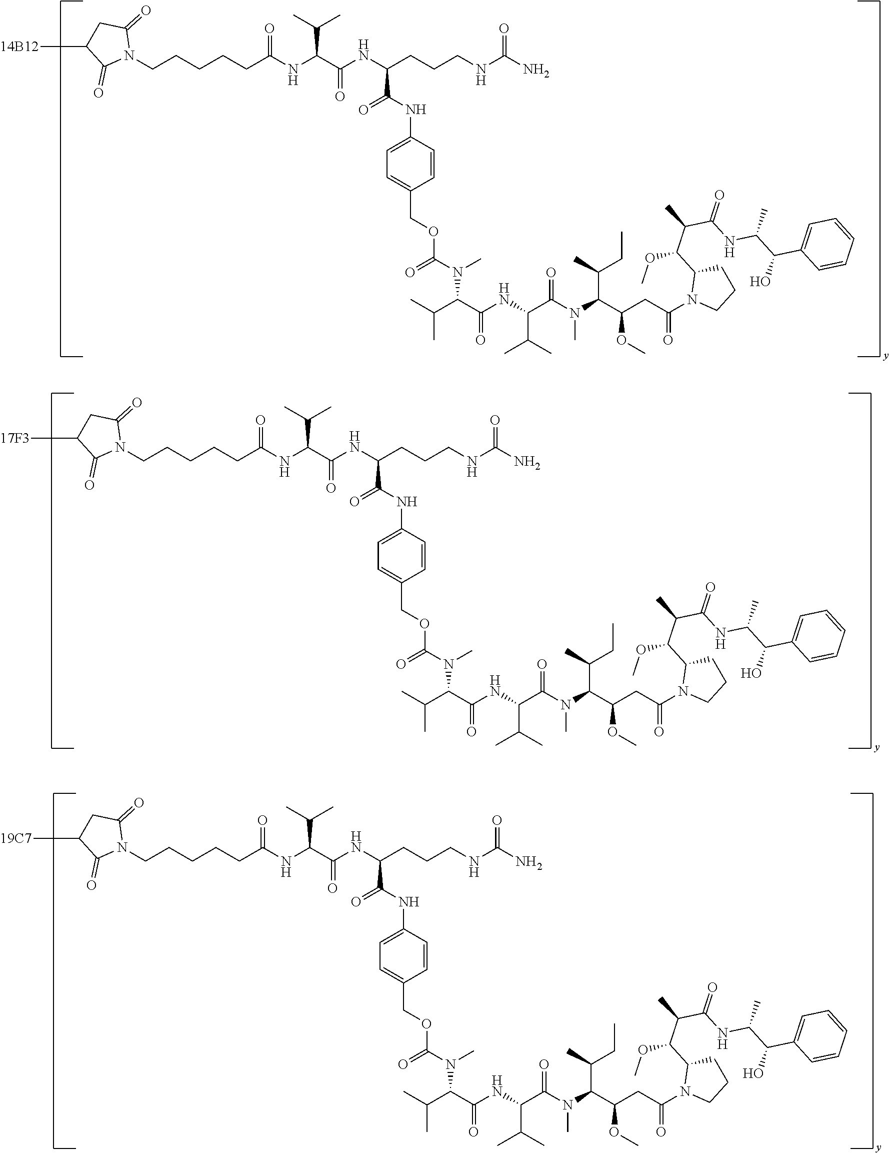

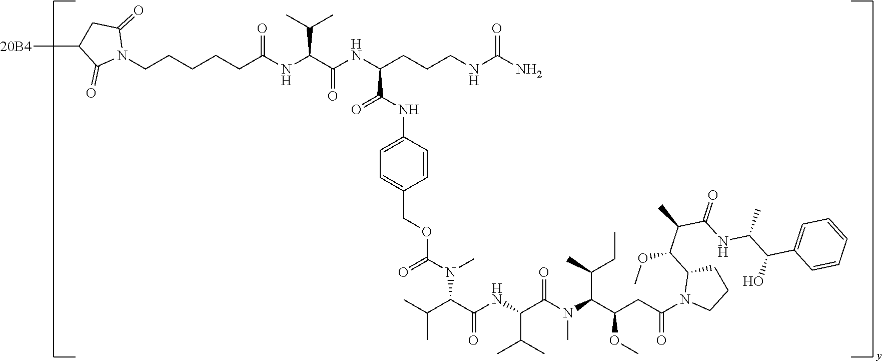

wherein: y is a number selected from 1 to 10, preferably a number selected from 2 to 8, more preferably a number of 4 to 8, further preferably a number of 4 to 6 or 6 to 8, and most preferably 4, 5, 6, 7 or 8.

In another aspect, the present disclosure provides a pharmaceutical composition comprising the antibody-drug conjugate according to any one of the aforementioned embodiments and one or more pharmaceutically acceptable excipients, diluents or carriers.

In another aspect, the present disclosure provides use of the antibody-drug conjugate according to any one of the aforementioned embodiments or a pharmaceutical composition comprising the same as a medicament.

In another aspect, the present disclosure provides use of the antibody-drug conjugate according to any one of the aforementioned embodiments or a pharmaceutical composition comprising the same in preparing a medicament for the treatment and/or prevention of a CDH17-mediated disease or condition.

In some embodiments of the present disclosure, the CDH17-mediated disease or condition is a tumor or cancer. The tumor or cancer is associated with high CDH17 expression. Or, the tumor or cancer is associated with moderate CDH17 expression.

In another aspect, the present disclosure provides use of the antibody-drug conjugate according to any one of the aforementioned embodiments or a pharmaceutical composition comprising the same in preparing a medicament for the treatment or prevention of a tumor or cancer; preferably the tumor or cancer is gastrointestinal cancer, pancreatic cancer, gallbladder cancer, cholangiocarcinoma, stomach cancer, intestinal cancer, ovarian cancer, colorectal cancer, lung cancer, breast cancer, anal cancer, prostate cancer, kidney cancer, bladder cancer, pharynx cancer, cancer of the nose, skin cancer, oral cancer, cancer of the tongue, esophageal cancer, vaginal cancer, cervical cancer, cancer of the spleen, testicular cancer, or glioblastoma.

In another aspect, the present disclosure further relates to a method for treating and/or preventing a tumor or cancer, wherein the method comprises administering to a patient in need thereof a therapeutically effective dose of the antibody-drug conjugate according to any one of the aforementioned embodiments or a pharmaceutical composition comprising the same; preferably the tumor or cancer is associated with high CDH17 expression, or the tumor or cancer is associated with moderate CDH17 expression.

In another aspect, the present disclosure further relates to a method for treating or preventing a tumor or cancer, wherein the method comprises administering to a patient in need thereof a therapeutically effective dose of the antibody-drug conjugate according to any one of the aforementioned embodiments or a pharmaceutical composition comprising the same; preferably the tumor or cancer is gastrointestinal cancer, pancreatic cancer, gallbladder cancer, cholangiocarcinoma, stomach cancer, intestinal cancer, ovarian cancer, colorectal cancer, lung cancer, breast cancer, anal cancer, prostate cancer, kidney cancer, bladder cancer, pharynx cancer, cancer of the nose, skin cancer, oral cancer, cancer of the tongue, esophageal cancer, vaginal cancer, cervical cancer, cancer of the spleen, testicular cancer, or glioblastoma.

The active compound (e.g., a ligand-drug conjugate according to the present disclosure, or the pharmaceutically acceptable salt or solvate thereof) may be formulated in a form suitable for administration by any suitable route, preferably in a form of a unit dose, or in a form of a single dose that can be self-administered by a subject. The unit dose of the present disclosure may be in a tablet, a capsule, a cachet, a vial, a powder, a granule, a lozenge, a suppository, a regenerating powder or a liquid formulation.

The administration dose of the active compound or composition used in the treatment method of the present disclosure will generally vary with the severity of the disease, the weight of the subject, and the efficacy of the active compound. However, as a general guide, a suitable unit dose may be 0.01 to 1000 mg.

The pharmaceutical composition of the present disclosure may comprise, in addition to the active compound, one or more excipients selected from the group consisting of: a filler, a diluent, a binder, a wetting agent, a disintegrating agent, an excipient and the like. Depending on the method of administration, the composition may comprise 0.01 to 99.9 wt. % of active compound.

### Advantageous Effects of the Invention

The CDH17 antibody and the antibody-drug conjugate provided by the present disclosure have good affinity for cell surface antigens, good endocytosis efficiency and high tumor inhibition efficiency as well as wider drug application windows, and are suitable for clinical drug application.

### BRIEF DESCRIPTION OF THE FIGURES

FIG. 1. In vitro binding characterization of hybridoma clones to AsPC1 (A), GP2d (B), SW480 (C) cells by flow cytometric analysis.

FIG. 2. Cell internalization activity of selected hybridoma clones were characterized using indirect killing assay.

FIG. 3. In vitro binding characterization of anti-CDH17 recombinant antibodies to AsPC1 (A) cells and SW480 (B) cells was determined by flow cytometric analysis, with HBMAB81 served as positive control and B12 as isotype control antibody (C).

FIG. 4. Dose-response curves of anti-CDH17 recombinant antibody internalization by indirect-killing assay.

FIG. 5. Binding competition: (A) 20B4; (B) HBMAB81.

FIG. 6. Dose-response curves of ADCs in vitro cytotoxicity activities on tumor cells.

FIG. 7. Comparison of the in vitro cytotoxicity of anti-CDH17 ADCs with different DAR values in GP2d cell line

FIG. 8. Comparison of the in vitro cytotoxicity of anti-CDH17 ADCs with different DAR values in AsPC1 cell line

FIG. 9. Comparison of the in vitro cytotoxicity of anti-CDH17 ADCs with different DAR values in SK-CO-1 cell line

FIG. 10. Comparison of the in vitro cytotoxicity of anti-CDH17 ADCs with different DAR values in AsPC1 cell line

FIG. 11. Anti-CDH17 ADCs inhibit tumor growth in AsPC1 tumor-bearing mice

FIG. 12. Anti-CDH17 ADCs inhibit tumor growth in GP2d tumor-bearing mice.

FIG. 13. Anti-CDH17 ADCs inhibit tumor growth in SNU16 tumor-bearing mice.

FIG. 14. Pharmacokinetic test of anti-CDH17 ADCs. Serum concentration levels of total antibody (A) and ADC (B)

FIG. 15. Pharmacokinetic test of anti-CDH17 ADCs. Serum concentration levels of total antibody and ADC

### DETAILED DESCRIPTION

### Definitions of Terms

The present invention is based on the development of an antibody which can specifically bind to CDH17. Antibodies of the present invention may optionally be conjugated to a growth inhibitory agent or cytotoxic agent such as a toxin, including, for example, a topoisomerase inhibitor or auristatin.

The titles used in the present section are for convenience of specification only, and do not limit the present invention. Unless otherwise defined herein, the scientific and technical terms used herein have the same meaning as commonly understood by those skilled in the art. Further, unless the context specifically requires, the singular includes the plural, and the plural includes the singular. The abbreviation of the amino acid residue is the standard three-letter and/or one-letter code used in the art, which represents one of the 20 common L-amino acids.

The terms “CDH17” and “CDH17 antigen” are used interchangeably herein, and include any variants, isoforms and species homologs of human CDH17 which are naturally expressed by cells or are expressed on cells transfected with the CDH17 gene.

“CDH17” as the target should be widely interpreted herein, aims to cover various forms of molecules of CDH17 at various stages in a mammalian (such as human), such as but not limited to molecules generated during the amplification, replication, transcription, splicing, translation, and modification of CDH17 gene (such as precursor CDH17, mature CDH17, membrane-expressed CDH17, CDH17 splicing variants, modified CDH17, or fragments thereof). This term also covers artificially prepared or in vitro expressed CDH17.

The term “antibody” or “antibodies” is meant in a broad sense and includes immunoglobulin molecules including monoclonal antibodies including murine, human, humanized and chimeric monoclonal antibodies, full-length antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity.

The “bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

The term “antibody” refers to a protein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hyper variability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e. g., effector cells) and the first component (C1q) of the classical complement system.

The term “antigen-binding fragment” of an antibody, as used herein, refers to one or more fragments of an antibody that retain the ability to specifically bind to an antigen (e. g., CDH17). It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL and CHI domains; (ii) a F (ab′) 2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341: 544-546), which consists of a VH domain; (vi) an isolated complementarity determining region (CDR), and (vii) a combination of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e. g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody.

The term “human antibody”, as used herein, is intended to include antibodies having variable and constant regions derived from human germline immunoglobulin sequences. The human antibodies of the invention may include amino acid residues not encoded by human germline immunoglobulin sequences (e. g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “recombinant human antibody”, as used herein, includes all human antibodies that are prepared, expressed, created or isolated by recombinant means, such as (a) antibodies isolated from an animal (e. g., a mouse) that is transgenic or trans chromosomal for human immunoglobulin genes or a hybridoma prepared therefrom (described further in Section I, below), (b) antibodies isolated from a host cell transformed to express the antibody, e. g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “CDR” refers to one of the six hypervariable regions within the variable domain of an antibody that primarily contributes to antigen binding. One of the most commonly used definitions for the six CDRs is provided by Kabat E. A. et al. (1991) Sequences of proteins of immunological interest. NIH Publication 91-3242. As used herein, the Kabat definition of CDR only applies to CDR1, CDR2 and CDR3 of the light chain variable domain (LCDR1, LCDR2, LCDR3 or L1, L2, L3), as well as CDR1, CDR2 and CDR3 of heavy chain variable domain (HCDR1, HCDR2, HCDR3 or H1, H2, H3).

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs including, e.g., Chothia based on the three-dimensional structure of antibodies and the topology of the CDR loops (Chothia et al. (1989) Nature 342:877-883), Kabat based on antibody sequence variability (Kabat et al., Sequences of Proteins of Immunological Interest, 4th edition, US Department of Health and Human Services, National Institutes of Health (1987)), AbM (University of Bath), Contact (University College London), International ImMunoGeneTics database (IMGT) (imgt.cines.fr/ on the World Wide Web), and North CDR definition based on the affinity propagation clustering using a large number of crystal structures. Those skilled in the art can easily identify the CDRs defined by each numbering system.

A useful comparison of CDR numbering is as below:

| CDR | IMGT | Kabat | AbM | Chothia¹ | Contact² |
|---|---|---|---|---|---|
| LCDR1 | 27-32 | 24-34 | 24-34 | 24-34 | 30-36 |
| LCDR2 | 50-51 | 50-56 | 50-56 | 50-56 | 46-55 |
| LCDR3 | 89-97 | 89-97 | 89-97 | 89-97 | 89-96 |
| HCDR1 | 26-35B | 31-35B | 26-35B | 26-32 . . . | 30-35B |
| | | (Kabat | | 34 | |
| | | Numbering)³ | | | |
| HCDR2 | 51-56 | 50-65 | 50-58 | 52-56 | 47-58 |
| HCDR3 | 93-102 | 95-102 | 95-102 | 95-102 | 93-101 |
| HCDR1 | 26-33 | 31-35 | 26-35 | 26-32 | 30-35 |
| | | (Chothia | | | |
| | | Numbering) | | | |

Note 1: some of these definitions (particularly for Chothia loops) vary depending on the individual publication examined; Note 2: any of the numbering schemes can be used for these CDR definitions, except the contact definition uses the Chothia or Martin (enhanced Chothia) definition; Note 3: the end of the Chothia HCDR1 loop when numbered using the Kabat numbering convention varies between H32 and H34 depending on the length of the loop. This is because the Kabat numbering scheme places the insertions at H35A and H35B.

The term “Fab fragment” includes a heavy chain variable domain and a light chain variable domain, and also includes the constant domain of the light chain and the first constant domain (CH1) of the heavy chain. An “Fab′ fragment” differs from the Fab fragment due to addition of some residues (including one or more cysteine from an antibody hinge region) to the carboxyl terminal of the heavy chain CH1 domain. “Fab′-SH” refers to an Fab′ in which the cysteine residue of the constant domain carries a free thiol group. An F (ab′) 2 antibody fragment was originally generated as paired Fab′ fragments with hinge cysteines between the Fab′ fragments. Other chemical couplings of antibody fragments are also known.

The term “Fc region” is used herein to define a C-terminal region of an immunoglobulin heavy chain, comprising at least a portion of a constant region. The term includes Fc regions and variant Fc regions of native sequences. In certain embodiments, a human IgG heavy chain Fc region extends from Cys226 or Pro230 to the carbonyl end of the heavy chain. However, the C-terminal lysine (Lys447) of the Fc region may or may not be present. Unless otherwise stated, the numbering of amino acid residues in the Fc region or constant region is based on an EU numbering system, which is also called EU index as described in Kabat et al., Sequences of Proteins of Immunological Interest, 5th Ed. Public Health Service, National Institutes of Health, Bethesda, Md., 1991.

As will be appreciated by those in the art, the exact numbering and placement of the heavy constant region domains can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service. National Institutes of Health, Bethesda, entirely incorporated by reference.

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

“Conservative modification” or “conservative replacement or substitution” or “conventional variant” refers to substitutions of amino acids in a protein with other amino acid having similar characteristics (e.g., charge, side chain size, hydrophobicity/hydrophilicity, backbone conformation and rigidity, etc.), such that the substitutions can be frequently made without altering the biological activity of the protein. It will be appreciated by those skilled in the art that, in general, a single amino acid substitution in a non-essential region of polypeptide does not substantially alter biological activity (see, for example, Watson et al. (1987) Molecular Biology of the Gene, The Benjamin/Cummings Pub. Co., Page 224, (4th edition)). In addition, substitutions with structurally or functionally similar amino acids are unlikely to affect biological activity.

The term “nucleic acid molecule” as used herein refers to a DNA molecule and a RNA molecule. The nucleic acid molecule may be single stranded or double stranded but is preferably a double stranded DNA. A nucleic acid is “effectively linked” when it is placed into functional relationship with another nucleic acid sequence. For example, if a promoter or enhancer affects transcription of a coding sequence, the promoter or enhancer is effectively linked to the coding sequence.

The preparation method of the nucleic acid is a conventional preparation method in the art. Preferably, it comprises the following steps: obtaining the nucleic acid molecule encoding the above-mentioned protein by gene cloning technology, or obtaining the nucleic acid molecule encoding the above-mentioned protein by the method of artificial full-length sequence synthesis.

Those skilled in the art know that the base sequence encoding the amino acid sequence of the protein can be replaced, deleted, changed, inserted or added appropriately to provide a polynucleotide homolog. The homolog of the polynucleotide of the present invention can be prepared by replacing, deleting or adding one or more bases of the gene encoding the protein sequence within the scope of maintaining the activity of the antibody.

The term “linking unit” as used herein means the part which links the antibody with the drug in the antibody-drug conjugate (i.e. ADC), which could be cleavable or uncleavable. The cleavable linker (i.e., breakable linker or biodegradable linker) may be broken in or on the target cells, and thereby releasing the drug. In some embodiments, the linking unit or linker of the present invention has very good stability and greatly decreases the release of the drug during the process of delivering to the target (e.g., in blood), thereby reducing the side effect and toxicity. In some particular embodiments, the linking unit or linker of the present invention is selected from cleavable linker, such as the linker based on disulphide (which is selectively broken in the tumor cells at a higher thiol concentration), peptide linker (which is cleaved by the enzyme in the tumor cells), and hydrazone linker.

The “linking unit” may comprise one or more linker elements. Exemplary linker elements include 6-maleimidocaproyl (“MC”), maleimidopropionyl (“MP”), valine-citrulline (“val-cit” or “vc”), alanine-phenylalanine (“ala-phe”), p-aminobenzyloxycarbonyl (“PAB”), N-succinimidyl 4-(2-pyridylthio) pentanoate (“SPP”), N-succinimidyl 4-(N-maleimidomethyl) cyclohexane-1 carboxylate (“SMCC”, also referred to herein as “MCC”), and N-succinimidyl (4-iodo-acetyl) aminobenzoate (“SIAB™”). The linker may comprise one or more of the following elements, or a combination thereof: a stretcher unit, a spacer unit and an amino acid unit, and may be synthesized by methods known in the art. The linker may be a “cleavable linker” favoring the release of drugs in cells. For example, acid-labile linkers (e.g., hydrazones), protease-sensitive (e.g., peptidase-sensitive) linkers, photolabile linkers, dimethyl linkers or disulfide-containing linkers can be used

(Chari et al, Cancer Research, 52: 127-131(1992); U.S. Pat. No. 5,208,020).

The term “toxin drug” or “cytotoxic drug” means a chemical molecule capable of strongly destructing normal growth in tumor cells. In principle, toxin drugs can kill tumor cells in high enough concentrations, but due to the lack of specificity, when killing tumor cells, it also leads to apoptosis in normal cells, leading to serious side effects. The cytotoxic drug may be selected from any agent that is detrimental to (e.g., kills) cells. Suitable cytotoxic agents for forming immunoconjugates of the present invention include but not limited to taxol, tubulysins, duostatins, cytochalasin B, gramicidin D, ethidium bromide, emetine, mitomycin, etoposide, tenoposide, vincristine, vinblastine, 48haracteri, doxorubicin, daunorubicin, dihydroxy anthracin dione, maytansine or an analog or derivative thereof, mitoxantrone, mithramycin, actinomycin D, 1 de¬hydro-testosterone, glucocorticoids, procaine, tetracaine, lidocaine, propranolol, and puromycin; calicheamicin or analogs or derivatives thereof; antimetabolites (such as methotrexate, 6 mercaptopurine, 6 thioguanine, cytarabine, 48haracteriz, 5 fluorouracil, 48haracteriz, hydroxyurea, asparaginase, gemcitabine, cladribine), alkylating agents (such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin; as well as duocarmycin A, duocarmycin SA, CC-1065 (a.k.a. rachelmycin), or analogs or derivatives of CC-1065), dolastatin, auristatin, pyrrolo[2,1-c][1,4]benzodiazepins (PDBs), indolinobenzodiazepine (IGNs) or analogues thereof, antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), anti-mitotic agents (e.g., tubulin-targeting agents), such as diphtheria toxin and related molecules (such as diphtheria A chain and active fragments thereof and hybrid molecules); ricin toxin (such as ricin A or a deglycosylated ricin A chain toxin), cholera toxin, a Shiga-like toxin (SLT I, SLT II, SLT IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca 48haracter proteins (PAPI, PAPII, and PAP S), 48haracter charantia inhibitor, curcin, crotin, Sapaonaria officinalis inhibitor, gelonin, mitogellin, restrictocin, phenomycin, and 48haracte toxins. Other suitable conjugated molecules include antimicrobial/lytic peptides such as CLIP, Magainin 2, mellitin, Cecropin, and P18; ribonuclease (Rnase), Dnase I, Staphylococcal enterotoxin A, pokeweed antiviral protein, diphtherin toxin, and Pseudomonas endotoxin.

In particular, the toxin drug may be selected from mitotic inhibitors, DNA alkylating agents, tyrosine kinase inhibitors, topoisomerase inhibitors, and DNA synthesis inhibitors, preferably tubulin inhibitors, topoisomerase inhibitors.

The term “alkyl” refers to a saturated aliphatic hydrocarbon group that is a linear or branched group containing 1 to 20 carbon atoms, preferably alkyl containing 1 to 12 carbon atoms, more preferably alkyl containing 1 to 10 carbon atoms, and most preferably alkyl containing 1 to 6 carbon atoms (containing 1, 2, 3, 4, 5 or 6 carbon atoms). Non-limiting examples include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and various side-chain isomers thereof, and the like. More preferred is a lower alkyl having 1 to 6 carbon atoms, and non-limiting examples include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl and the like. Alkyl may be substituted or unsubstituted. When substituted, the substituent may be substituted at any available connection site, wherein the substituent is preferably one or more of the following groups independently selected from the group consisting of alkyl, alkenyl, alkynyl, alkoxy, alkylthio, alkylamino, halogen, mercapto, hydroxy, nitro, cyano, cycloalkyl, heterocycloalkyl, aryl, heteroaryl, cycloalkoxy, heterocycloalkoxy, cycloalkylthio, heterocycloalkylthio and oxo.

In some aspect, anti-CDH17 antibodies may be conjugated to one or more topoisomerase inhibitors(s) to form an ADC for the treatment of cancer. Topoisomerases are enzymes that are capable of altering DNA topology in eukaryotic cells. They are critical for cellular functions and cell proliferation. In some aspects, the topoisomerase inhibitor is camptothecin or a camptothecin analog. Camptothecin is a water-insoluble, cytotoxic alkaloid produced by Camptotheca accuminata trees indigenous to China and Nothapodytes foetida trees indigenous to India. Camptothecin exhibits tumor cell growth inhibiting activity against a number of tumor cells. Compounds of the camptothecin analog class are typically specific inhibitors of DNA topoisomerase I. By the term “topoisomerase inhibitor” is meant any tumor cell growth inhibiting compound that is structurally related to camptothecin.

In some embodiments, the camptothecin analog is an active metabolite of irinotecan (CPT-11). In some such embodiments, the camptothecin analog is 7-ethyl-10-hydroxycamptothecin (SN-38). As a metabolite, SN-38 is formed by hydrolysis of irinotecan by carboxylesterases. In some embodiments, the camptothecin analog is exatecan methanesulfonate. Exatecan methanesulfonate is a water-soluble camptothecin (CPT) that exhibits more potent topoisomerase I inhibitory activity and antitumor activity than other CPT analogs. In addition, exatecan is effective against p-glycoprotein (P-gp)-mediated multi-drug resistant cells. In some embodiments, the camptothecin analog is deruxtecan (Dxd), a potent derivative of exatecan, which has 10-fold higher topoisomerase I inhibitory potency than SN-38.

The term “ligand-cytotoxicity drug conjugate” means that a ligand is linked to a biologically active drug through a linking unit. In some particular embodiments, the “ligand-cytotoxicity drug conjugate” is preferably an antibody-drug conjugate (ADC), which means that a monoclonal antibody or antibody fragment is linked to a cytotoxic drug with biological activity through a linking unit.

The term “Drug to Antibody Ratio (DAR)” means the average number of cytotoxic drugs loaded on each ligand, and can also be represented as ratio of drug amount and antibody amount. The range of drug loading for each ligand (Ab) can be 1-20 cytotoxic drugs (D). In the embodiment of the present invention, the Drug to Antibody Ratio is represented as y. The average number of drugs in each ADC molecule after the coupling reaction can be identified by conventional methods, such as UV/visible spectroscopy, mass spectrometry, ELISA test, and HPLC characteristic identification.

The term “transfectoma”, as used herein, includes recombinant eukaryotic host cell expressing the antibody, such as CHO cells, NS/0 cells, HEK293 cells, plant cells, or fungi, including yeast cells.

The sequence of the DNA molecule for the antibody or a fragment thereof according to the present invention can be obtained by conventional techniques, for example, methods such as PCR amplification or genomic library screening. In addition, the sequences encoding light chain and heavy chain can be fused together, to form a single-chain antibody.

Once a relevant sequence is obtained, the relevant sequence can be obtained in bulk using a recombination method. This is usually carried out by cloning the sequence into a vector, transforming a cell with the vector, and then separating the relevant sequence from the proliferated host cell by conventional methods.

In addition, a relevant sequence can be synthesized artificially, especially when the fragment is short in length. Usually, several small fragments are synthesized first, and then are linked together to obtain a fragment with a long sequence.

At present, it is possible to obtain a DNA sequence encoding the antibody of the present invention (or fragments thereof, or derivatives thereof) completely by chemical synthesis. The DNA sequence can then be introduced into a variety of existing DNA molecules (or, for example, vectors) and cells known in the art. In addition, mutations can also be introduced into the protein sequences of the present invention by chemical synthesis.

In general, under conditions suitable for expression of the antibody according to the present invention, the host cell obtained is cultured. Then, the antibody of the present invention is purified by using conventional immunoglobulin purification steps, for example, the conventional separation and purification means well known to those skilled in the art, such as protein A-Sepharose, hydroxyapatite chromatography, gel electrophoresis, dialysis, ion exchange chromatography, hydrophobic chromatography, molecular sieve chromatography or affinity chromatography.

The monoclonal antibody obtained can be identified by conventional means. For example, the binding specificity of a monoclonal antibody can be determined by immunoprecipitation or an in vitro binding assay (such as radioimmunoassay (RIA) or enzyme-linked immunosorbent assay (ELISA)). The binding affinity of a monoclonal antibody can be determined by, for example, the Scatchard analysis (Munson et al., Anal. Biochem., 107: 220 (1980)).

The antibody according to the present invention can be expressed in a cell or on the cell membrane, or is secreted extracellularly. If necessary, the recombinant protein can be separated and purified by various separation methods according to its physical, chemical, and other properties. These methods are well known to those skilled in the art. The examples of these methods comprise, but are not limited to, conventional renaturation treatment, treatment by protein precipitant (such as salt precipitation), centrifugation, cell lysis by osmosis, ultrasonic treatment, supercentrifugation, molecular sieve chromatography (gel chromatography), adsorption chromatography, ion exchange chromatography, high performance liquid chromatography (HPLC), and any other liquid chromatography, and the combination thereof.

The term “identity” of sequences refers to the relationship between the sequences of two or more polypeptide molecules or two or more nucleic acid molecules, as determined by aligning sequences. Generally, identity refers to the number or percentage of identical positions shared by two amino acid or nucleic acid sequences, taking into account the number of gaps, and the length of each gap, which need to be introduced for optimal alignment of the two sequences. Generally, before calculating the percentage of identity between two amino acid or nucleotide sequences, sequence alignment is performed, and gap (if any) is introduced. If the amino acid residues or bases in the two sequences are the same at a certain alignment position, the two sequences are considered to be identical or matched at that position. If the amino acid residues or bases in the two sequences are different, it is considered that they are inconsistent or mismatched at this position. In some algorithms, the number of matching positions is divided by the total number of positions in the alignment window to obtain sequence identity. In other algorithms, the number of notches and/or the length of notches are also taken into account. For the purpose of the present disclosure, the well-known alignment software BLAST (which can be found on the web page ncbi.nlm.nih.gov) can be used to obtain the best sequence alignment and calculate the sequence identity between the two amino acid or nucleotide sequences by using the default settings. When an amino acid sequence is described as being at least 85% or at least 90% or at least 95% identical to another amino acid sequence, the difference in the amino acid sequence may lie in conservative substitution (including all substitutions therein are conservative substitutions).

The term “variant” of a polypeptide such as for example, an antigen-binding fragment, a protein, or an antibody is a polypeptide in which one or more amino acid residues are inserted, deleted, added, and/or substituted, as compared to another polypeptide sequence, and includes a fusion polypeptide. In addition, a protein variant includes one modified by protein enzyme cutting, phosphorylation, or other posttranslational modification, but maintaining biological activity of the antibody disclosed herein, for example, binding to CDH17 and specificity. The variant may be about 99%, 98%, 97%, 96%, 95%, 94%, 93%, 92%, 91%, 90%, 89%, 88%, 87%, 86%, 85%, 84%, 83%, 82%, 81%, or 80% identical to the sequence of the antibody or its antigen-binding fragment disclosed herein. The percent identity (%) or homology may be calculated with reference to the following description.

In one embodiment, the percent homology or identity may be calculated as 100×[(identical position)/min (TGA, TGB)], and in the formula, TGA, TGB are the sum of the number of residues of sequences A and B compared and the internal gap position (Russell et al., J. Mol Biol., 244: 332-350 (1994).

In the present invention, the antibody of the present invention also includes a conservative variant thereof, which means that, compared to the amino acid sequence of the antibody of the present invention, there are up to 10, preferably up to 8 and more preferably up to 5, most preferably up to 3 amino acids are replaced by amino acids with similar or similar properties to form a polypeptide. These conservative variant polypeptides are preferably produced by amino acid substitution according to Table A.

TABLE A

| Original | Representative | Preferred |
|---|---|---|
| residue | replacement | replacement |
| Ala | Val; Leu; Ile | Val |
| Arg | Lys; Gln; Asn | Lys |
| Asn | Gln; His; Lys; Arg | Gln |
| Asp | Glu | Glu |
| Cys | Ser | Ser |
| Gln | Asn | Asn |
| Glu | Asp | Asp |
| Gly | Pro; Ala | Ala |
| His | Asn; Gln; Lys; Arg | Arg |
| Ile | Leu; Val; Met; Ala; Phe | Leu |
| Leu | Ile; Val; Met; Ala; Phe | Ile |
| Lys | Arg; Gln; Asn | Arg |
| Met | Leu; Phe; Ile | Leu |
| Phe | Leu; Val; Ile; Ala; Tyr | Leu |
| Pro | Ala | Ala |
| Ser | Thr | Thr |
| Thr | Ser | Ser |
| Trp | Tyr; Phe | Tyr |
| Tyr | Trp; Phe; Thr; Ser | Phe |
| Val | Ile; Leu; Met; Phe; Ala | Leu |

The term “K_{D}” (M), as used herein, is intended to refer to the dissociation equilibrium constant of a particular antibody-antigen interaction. “K_{D}” refers to the dissociation constant, which is obtained from the ratio of K_{d} to Kₐ (i.e., K_{d}/Kₐ) and is expressed as a molar concentration (M). K_{D} values for antibodies can be determined using methods in the art in view of the present disclosure. For example, the K_{D} of an antibody can be determined by using surface plasmon resonance, such as by using a biosensor system, e.g., a system, or by using bio-layer interferometry technology, such as an Octet RED96 system.

The term “affinity” is the strength of interaction between an antibody or its antigen-binding fragment and an antigen, and it is determined by properties of the antigen such as size, shape and/or charge of antigen, and CDR sequences of the antibody or antigen-binding fragment. The methods for determining the affinity are known in the art, and the followings can be referred.

The antibody or its antigen-binding fragment is called “specifically binding” to its target such as an antigen, when a dissociation constant (K_{D}) is <10⁻⁶ M. The antibody specifically binds to a target with “high affinity”, when K_{D} is <10⁻⁹ M.

The term “Pharmaceutical composition”, as used herein, is intended to refer to a mixture containing one or more of the compounds or a physiological/pharmaceutically acceptable salt or prodrug thereof described herein with other chemical components, such as physiological/pharmaceutically acceptable carriers and excipients. The purpose of the pharmaceutical composition is to promote the administration to the organism, which is beneficial to the absorption of the active ingredient and exerts the biological activity.

“Administration” and “treatment”, when applied to an animal, human, experimental subject, cell, tissue, organ, or biological fluid, refer to contact with an exogenous pharmaceutical, therapeutic, diagnostic reagent, or composition with the animal, human, subject, cell, tissue, organ, or biological fluid. “Administration” and “treatment” can refer, e.g., to therapeutic, pharmacokinetic, diagnostic, research, and experimental methods. Treatment of a cell encompasses contacting the cell with a reagent, as well as contacting a fluid with a reagent, wherein the fluid is in contact with the cell. “Administration” and “treatment” also mean in vitro and ex vivo treatments, e.g., of a cell, by a reagent, diagnostic, binding composition, or by another cell. “Treatment,” when applied to a human, veterinary, or research subject, refers to therapeutic treatment, prophylactic or preventative measures, research, and diagnostic applications.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

The present disclosure also comprises various deuterated forms of the compounds of formula (I) and (A). Each available hydrogen atom connected to a carbon atom may be independently substituted with a deuterium atom. Those skilled in the art are able to synthesize the compounds of formula (I) and (A) in deuterated form with reference to the relevant literature. Commercially available deuterated starting materials can be used in preparing the deuterated forms of the compounds of formula (I) and (A), or they can be synthesized using conventional techniques with deuterated reagents including, but not limited to, deuterated borane, tri-deuterated borane in tetrahydrofuran, deuterated lithium aluminum hydride, deuterated iodoethane, deuterated iodomethane, and the like.

In addition, the present disclosure includes a medicament for treating a disease associated with CDH17, comprising an antibody, an antigen-binding fragment, or an antibody-drug conjugate thereof of the present disclosure as an active ingredient.

There is no limitation on the diseases related to CDH17, as long as it is a disease associated with CDH17, for example, the therapeutic response induced by the molecules disclosed in the present disclosure can be reduced by binding human CDH17. Therefore, the molecules of the present disclosure are very useful for those who suffer from a tumor, cancer, or infectious disease when in preparations and formulations suitable for therapeutic applications.

In addition, the present disclosure relates to a method for immunologically detecting or measuring CDH17, a reagent for immunologically detecting or measuring CDH17, a method for immunologically detecting or measuring cells expressing CDH17, and a diagnostic reagent for diagnosis of disease related to CDH17-positive cells, comprising the antibody or antigen-binding fragment of the present disclosure that specifically recognizes human CDH17, as an active ingredient.

In the present disclosure, the method for detecting or determining the amount of CDH17 may be any known method. For example, it includes immune-detection or assay.

The immune-detection or assay is a method of detecting or determining the amount of antibody or antigen by using labeled antigen or antibody. Examples of immune-detection or assay include a radioactive substance labeled immunological antibody method (MA), an enzyme immunoassay (EIA or ELISA), a fluorescent immunoassay (FIA), a luminescent immunoassay, a western blotting method, physicochemical methods, etc.

The above-mentioned diseases related to CDH17-positive cells can be diagnosed by detecting or measuring cells expressing CDH17 by using the antibodies or antibody fragments thereof of the present invention.

In order to detect cells expressing the polypeptide, a known immune-detection can be used, and preferably immunoprecipitation, fluorescent cell staining, or immunohistochemically staining, etc. can be used. Furthermore, a fluorescent antibody staining method, etc. using FMAT8100HTS system (Applied Bio system) can be used.

### EXAMPLES

The invention is further illustrated by the following specific examples. It is to be understood that these examples are for illustrative purposes only and are not intended to limit the scope of the invention. The experimental methods without detailed conditions in the following examples are generally in accordance with the conditions described in the conventional conditions such as Sambrook. J et al. “Guide to Molecular Cloning Laboratory” (translated by Huang Peitang et al., Beijing: Science Press, 2002), or in accordance with the conditions recommended by the manufacturer (for example, product manuals). Percentages and parts are by weight unless otherwise stated. The experimental materials and reagents used in the following examples are commercially available unless otherwise specified.

The room temperature described in the examples is a conventional room temperature in the art, and is generally 10-30° C.

### Example 1 Mouse Immunization and Hybridoma Fusion

### Immune Protocol:

Anti-CDH17 antibodies were obtained by immunizing genetically modified mouse encoding human immunoglobulin heavy and kappa light chain variable regions with recombinant protein antigen human CDH17 His Tag (Sino biological, catalog number 11360-H08H) and boosted with the same antigen for 2 times. The antibody immune response was monitored by a CDH17-specific immunoassay. When a desired immune response was achieved splenocytes were harvested from each mouse and fused with mouse myeloma cells to preserve their viability and form hybridoma cells and screened for CDH17 specificity.

### Spleen Cell Fusion

The spleen lymphocytes and myeloma cells Sp2/0 (ATCC® CRL-158) were fused to obtain hybridoma cells by electrofusion or PEG fusion. PEG fusion was performed using Clonacell™ HY technology (STEMCELL technologies), following the manufacturer's instructions. The primary cell: mouse myeloma cell line ratio was 1:1 for electrofusion, and 10:1 for PEG fusion.

### Example 2 Hybridoma Screening

### Screening of Hybridoma Clones Specifically Binding to Human CDH17 Protein by ELISA

ELISA was performed using the DuoSet ELISA Ancillary Kit (R&D System, DY008). ELISA plates were coated with 1 μg/ml of human CDH17 His Tag (Sino biological, catalog number 11360-H08H), or BSA overnight. Excess unbound proteins were washed off by washing the plates three times with the wash buffer before blocking for 1 hour at room temperature. 100 μl CDH17 hybridoma supernatant was added in each well and incubated for 1 hour at room temperature. Excess unbound antibodies were washed off and 100 μl of 1:30000 diluted secondary antibody Goat anti-mouse IgG Fc-HRP (ab5870) was added to each well for another 1 hour. Plates were washed before the addition 50 μL of chemiluminescence agents (color A and color B) according to manufacturer's protocol. The reactions were stopped using 25 μL of stop solution. Optical density at 450 nm of samples was measured by a microplate reader (PerkinElmer). All tested clones bound selectively to human CDH17 but not BSA.

TABLE 1
Binding characterization of hybridoma clones
to human CDH17 protein by ELISA assay (OD450)

| | Protein binding | Protein binding |
|---|---|---|
| Clone ID | (human CDH17) | (BSA) |
| 1H10 | 1.7895 | 0.0399 |
| 2F9 | 1.7237 | 0.0718 |
| 4F9 | 1.5202 | 0.051 |
| 9E2 | 1.4804 | 0.0429 |
| 10E11 | 1.4809 | 0.0475 |
| 13C7 | 1.6185 | 0.0432 |
| 14B12 | 1.8849 | 0.041 |
| 15A4 | 1.6026 | 0.0431 |
| 17F3 | 1.663 | 0.0425 |
| 19C7 | 1.8366 | 0.0468 |
| 20B4 | 1.8098 | 0.0406 |

### Screening of Hybridoma Clones Specifically Binding to CDH17 Expressing Cancer Cells by Flow Cytometry

Hybridoma supernatants were subjected to binding tests on CDH17 positive cell line AsPC1 (ATCC, CRL-1682), GP2d (Creative bioarray, CSC-J9456), and CDH17 negative cell line SW480 (ATCC, CCL-228) using flow cytometry analysis. Briefly, 50 μL of cells in cell staining buffer (2×10⁶ cells/mL) was mixed with 50 μL undiluted supernatants. The mixture was incubated on ice for 1 hour and then washed with ice-cold staining buffer twice. The cells were subsequently stained with 50 μL of PE labeled secondary antibody (1:250 dilution, Biolegend, Cat #405307) for 20 min. After washing by staining buffer and fixing by 4% PFA, cells were analyzed by flow cytometry.

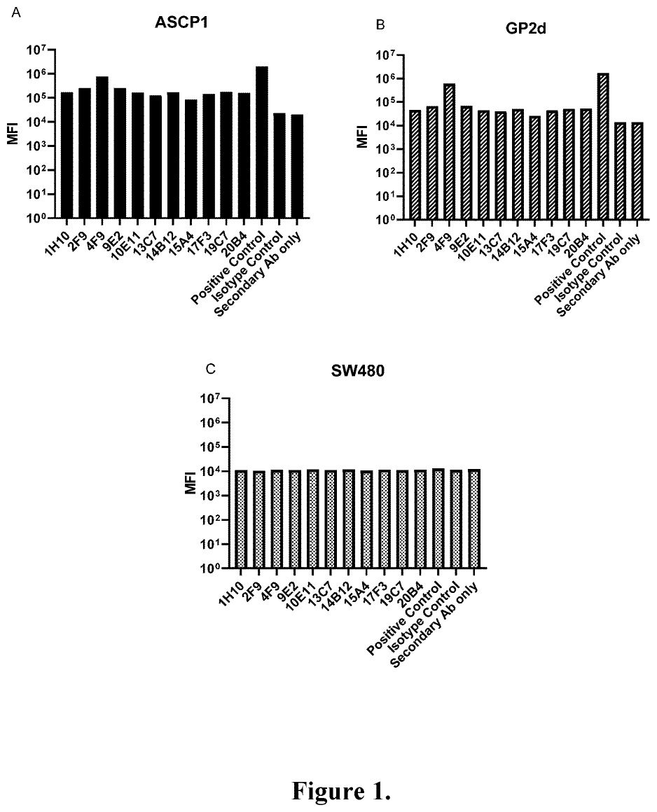

A purified anti-human CDH17 antibody was used as a positive control (Sino Biological, Cat #11360-MM02). A purified mouse IgG1 antibody was used as isotype control (Biolegend, Cat #400102). FIG. 1 shows examples of selected cell binding signals measured by flow cytometry. The hybridoma clones 1H10, 2F9, 4F9, 9E2, 10E11, 13C7, 14B12, 15A4, 17F3, 19C7, 20B4 were identified with enhanced binding profile to AsPC1 cells and GP2d cells compared to SW480 cells.

Screening of Hybridoma Clones with Cell Internalization Activity by Indirect Killing Assay

Cell internalization activities of hybridoma supernatants were measured using an indirect killing assay in AsPC1 cells. The AsPC1 cells were seeded in 96 well plate at 8,000 cells/well with propidium iodide (abeam, Cat #ab14083) at 500 ng/mL and SPY650-DNA (Cytoskeleton, Inc., Cat #CYSC501) at 1:2000 dilution. Cells were incubated overnight at 37° C. in 5% CO₂. The hybridoma supernatants from each hybridoma clone were diluted with hybridoma culture medium containing 500 ng/mL propidium iodide and 2000-fold diluted SPY650-DNA and mixed with Fab anti-mouse IgG Fc-MMAF conjugates with cleavable linker (Moradec, AM-202AF), then added to each well. The final concentrations of mouse IgG were roughly 10 nM, 3.33 nM, and 1.11 nM. The final concentration of the Fab anti-mouse IgG Fc-MMAF conjugates in each well was 20 nM. With the presence of secondary Fab-vc-MMAF, the internalized antibody/Fab-vc-MMAF conjugates complex will release the cytotoxic payload and kill the cells. Cytation5 (Agilent) was used to detect the viability of the cells in each well by imaging the plates every 8 h.

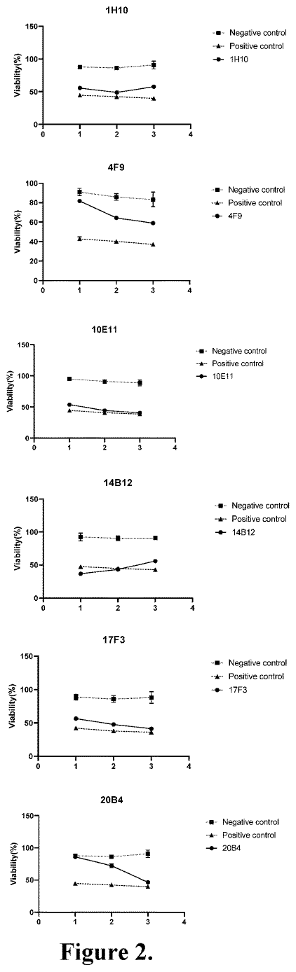

A purified anti-human CDH17 antibody was used as a positive control (Invitrogen, Cat #MA5-29135). A purified mouse IgG1 antibody was used as isotype control (Biolegend, Cat #400102). Cells treated with selected hybridoma supernatants showed reduced viability, as shown in FIG. 2, indicating the internalization of the antibodies. The clones 1H10, 2F9, 4F9, 9E2, 10E11, 13C7, 14B12, 15A4, 17F3, 19C7, 20B4 were identified to be capable of internalizing into CDH17 positive cell line using the indirect killing assay.

### Example 3 Sequencing of Positive Hybridoma Clones

The process of cloning sequences from positive hybridomas are as follows. The logarithmic growth phase hybridoma cells were collected, RNA was extracted, and reverse transcription was performed then followed by VDJ region amplification. Amplified cDNA library from each clone were subjected to next-generation sequencing. The amino acid sequences of the heavy and light chain variable region DNA sequences corresponding to the antibodies 1H10, 2F9, 4F9, 9E2, 10E11, 13C7, 14B12, 15A4, 17F3, 19C7, 20B4 were obtained. The amino acid sequence of heavy chain variable and light chain variable regions and CDR sequence of each antibody are as the following tables (Table 2 to 4). The amino acid residues of the CDRs in VH/VL are numbered and annotated according to the Kabat numbering system.

TABLE 2
CDR sequence of heavy chain variable domain for CDH17 hybridoma clones.

| | CDR Sequence of Heavy Chain Variable (VH) | | | | | |
|---|---|---|---|---|---|---|
| | CDR1 | | CDR2 | | CDR3 | |
| | | SEQ | | SEQ | | SEQ |
| | | ID | | ID | | ID |
| Clone | Sequence | NO | Sequence | NO | Sequence | NO |
| 1H10 | SYGMH | 1 | FIWYDGSNKN | 11 | DRYSSGWDV | 22 |
| | | | YADSVKG | | FDY | |
| 2F9 | DYGMS | 2 | GLSWNGGNTY | 12 | GSRYNWNYD | 23 |
| | | | YADSVKG | | AFDI | |
| 4F9 | SSNWWS | 3 | EIYHSGSTSYNP | 13 | NDWGFPLDC | 24 |
| | | | SLKS | | | |
| 9E2 | NYGMS | 4 | GINWNGGSTY | 14 | GSRYNWSYD | 25 |
| | | | YADSVRG | | AFDI | |
| 10E11 | SYWIG | 5 | IIYPGDSDTRYS | 15 | RGWVNYFDY | 26 |
| | | | PSFQG | | | |
| 13C7 | TSGVGVG | 6 | LIYWNDDKRY | 16 | YGVTAPFYN | 27 |
| | | | RPSLKN | | WFDP | |
| 14B12 | TYYRS | 7 | YIYYSGNTNYN | 17 | AQYGEEAFDI | 28 |
| | | | PSLKS | | | |
| 15A4 | SYGMH | 1 | FVWYDGSNQN | 18 | DRYSSGWDV | 22 |
| | | | YADSVKG | | FDY | |
| 17F3 | SYSLK | 8 | SISSSSGYIYYA | 19 | AVSTIAARPG | 29 |
| | | | DSVKG | | YYYYYMDV | |
| 19C7 | TGGVGVG | 9 | LIYWNDDKRYS | 20 | SGLAAPFYN | 30 |
| | | | PSLKS | | WFDP | |
| 20B4 | EVFMH | 10 | GFDPEDGETIY | 21 | GWGSRYFDY | 31 |
| | | | AQKFQG | | | |

TABLE 3
CDR sequence of light chain variable domain for CDH17 hybridoma clones.

| | CDR Sequence of Light Chain Variable (VL) | | | | | |
|---|---|---|---|---|---|---|
| | CDR1 | | CDR2 | | CDR3 | |
| | | SEQ | | SEQ | | SEQ |
| | | ID | | ID | | ID |
| Clone | Sequence | NO | Sequence | NO | Sequence | NO |
| 1H10 | RASQDISSWLA | 32 | AASSLQS | 43 | QQAKSFPPT | 48 |
| 2F9 | RASQGIRNALG | 33 | AASNLQS | 44 | LQHNSYPFT | 49 |
| 4F9 | RASQSISSYLN | 34 | AASSLQS | 43 | QQSHNIPIT | 50 |
| 9E2 | RASQGVRNALG | 35 | AASSLQS | 43 | LQHNSYPFT | 49 |
| 10E11 | RASQGISNWLG | 36 | AASSLQS | 43 | QQANSFPLT | 51 |
| 13C7 | RASQSITSYLN | 37 | AASSLQS | 43 | QRSYSTLT | 52 |
| 14B12 | RASQSITTYLN | 38 | AASRLQT | 45 | QQSYNTPPT | 53 |
| 15A4 | RASQGISSWLA | 39 | AASSLQS | 43 | QQAKSFPPT | 48 |
| 17F3 | RASQDISTWFA | 40 | AASTLRS | 46 | QQANSFPLT | 51 |
| 19C7 | RASQSISNYLN | 41 | AASSLQS | 43 | QRSYSTLT | 52 |
| 20B4 | KSSQSVLYSSNN | 42 | WASTRES | 47 | HQYYSTPLT | 54 |
| | KNYLA | | | | | |

TABLE 4
Sequences of heavy chain and light chain variable domains for CDH17 hybridoma
clones.

| | Sequence of Heavy Chain Variable | | Sequence of Light Chain Variable | |
|---|---|---|---|---|
| | | SEQ | | SEQ |
| Clone | Sequence | ID NO | Sequence | ID NO |
| 1H10 | QVQLVESGGGVVQPGRSL | 55 | DIQMTQSPSSVSASVGDRV | 66 |
| | RLSCVASGFTFSSYGMHW | | TITCRASQDISSWLAWYQQ | |
| | VRQAPGKGLEWVTFIWYD | | QPGKAPKLQIYAASSLQSG | |
| | GSNKNYADSVKGRFTISRD | | VPSRFSGSGSGTDFTLTISS | |
| | NSKNTLNLQMNSLRVEDT | | LQPEDFATYYCQQAKSFPP | |
| | AVYYCARDRYSSGWDVFD | | TFGQGTKVEIK | |
| | YWGQGTLVTVSS | | | |
| 2F9 | EVQLVESGGGVVRPGGSL | 56 | DIQMTQSPSSLSASIGDRVT | 67 |
| | RLSCAASGFTFDDYGMSW | | ITCRASQGIRNALGWYQQ | |
| | VRQAPGKGLEWVSGLSW | | KPGKAPKRLIYAASNLQSG | |
| | NGGNTYYADSVKGRFTISR | | VPSRFSGSGSGTEFTLTISSL | |
| | DNAKNSLYLQMNSLRAED | | QPEDFATYYCLQHNSYPFT | |
| | TALYYCARGSRYNWNYDA | | FGPGTKVEIK | |
| | FDIWGQGTLVTVSS | | | |
| 4F9 | QVQLQESGPGLVKPSGTLS | 57 | DIQMTQSPSSLSASVGDRV | 68 |
| | LTCAVSGGSISSSNWWSWV | | TITCRASQSISSYLNWYQQ | |
| | RQPPGKGLEWIGEIYHSGS | | KPGKAPKLLIYAASSLQSG | |
| | TSYNPSLKSRVTISVDKSK | | VPSRFSGSGSGTHFTLTISS | |
| | NQFSLKLSSVTAADTAVYY | | LQPEDFATYYCQQSHNIPIT | |
| | CARNDWGFPLDCWGQGT | | FGQGTRLEIK | |
| | LVTVSS | | | |
| 9E2 | EVQLVESGGGVVRPGGSL | 58 | DIQMTQSPSSLSASVGDRV | 69 |
| | RLSCAASGFTFDNYGMSW | | TITCRASQGVRNALGWYQ | |
| | VRQAPGKGLEWVSGINWN | | QKPGKAPKRLIYAASSLQS | |
| | GGSTYYADSVRGRFTISRD | | GVPSRFSGSGSGTEFTLTIS | |
| | NAKNSLYLQMNSLRAEDT | | SLQPEDFATYYCLQHNSYP | |
| | ALYYCARGSRYNWSYDAF | | FTFGPGTKVEIK | |
| | DIWGQGTLVTVSS | | | |
| 10E11 | EVQLVQSGTEVKKPGESLK | 59 | DIQMTQSPSSVSASVGDRV | 70 |
| | ISCKDSGYRFSSYWIGWVR | | TITCRASQGISNWLGWYQ | |
| | QMPGKGLEWMGIIYPGDS | | QKPGKAPKLLIYAASSLQS | |
| | DTRYSPSFQGQVTISADKSI | | GVPSRFSGSGSGTDFTLTIS | |
| | STAYLQWRSLKASDTAMY | | SLQPEDFATYYCQQANSFP | |
| | YCARRGWVNYFDYWGQG | | LTFGGGTKVEIK | |
| | TLVTVSS | | | |
| 13C7 | QITLKESGPTLVKPTQTLTL | 60 | DIQMTQSPSSLSASVGDRV | 71 |
| | TCTFSGFSLSTSGVGVGWI | | TITCRASQSITSYLNWYQQ | |
| | RQPPGKALEWLALIYWND | | KPGKAPKLLIYAASSLQSG | |
| | DKRYRPSLKNRLTITKDTS | | VPSRFSGSGSGTDFTLTISS | |
| | KNQVVLTMTNMDPVDTAT | | LQPEDFATYYCQRSYSTLT | |
| | YYCTHYGVTAPFYNWFDP | | FGGGTKVEIK | |
| | WGQGTLVTVSS | | | |
| 14B12 | QVQLQESGPGLVKPSETLS | 61 | DIQMTQSPSSLSASVGDRV | 72 |
| | LTCTVSGGSINTYYRSWIR | | TITCRASQSITTYLNWYQQ | |
| | QPPGKGLEWIGYIYYSGNT | | KPGKAPQLLIFAASRLQTG | |
| | NYNPSLKSRVTISADTSKN | | VPSRFSGSGSGTDFTLTISS | |
| | QFSLNLISVTAADTAMYYC | | LLPGDFATYYCQQSYNTPP | |
| | ARAQYGEEAFDIWGQGTL | | TFGGGTKVEIK | |
| | VTVSS | | | |
| 15A4 | QVQLVESGGGVVQPGRSL | 62 | DIQMTQSPSSVSASVGDRV | 73 |
| | RLSCAASGFNFSSYGMHW | | TITCRASQGISSWLAWYQQ | |
| | VRQAPGKGLEWVAFVWY | | KPGKAPKLLIFAASSLQSG | |
| | DGSNQNYADSVKGRFTISR | | VPSRFSGSGSGTGFTLTISS | |
| | DNSNNTLYLQMNSLRAED | | LQPEDFATYYCQQAKSFPP | |
| | TAVYYCARDRYSSGWDVF | | TFGQGTKVEIK | |
| | DYWGQGTLVTVSS | | | |
| 17F3 | EVQLVESGGGLVKSGGSLR | 63 | DIQMTQSPSSVSASIGDRVT | 74 |
| | LSCAASGFTFSSYSLKWVR | | ITCRASQDISTWFAWYQQK | |
| | QAPGKGLEWVSSISSSSGYI | | PGKAPKLLIYAASTLRSGV | |
| | YYADSVKGRFTISRDNAKN | | PSRFSGSGSGTDFTLTINSL | |
| | SLYLQMNSLRAEDTAMYY | | QPEDFATYYCQQANSFPLT | |
| | CARAVSTIAARPGYYYYY | | FGGGTKVEIK | |
| | MDVWGKGTLVTVSS | | | |
| 19C7 | QITLKESGPTLVKPTQTLTL | 64 | DIQMTQSPSSLSASVGDRV | 75 |
| | TCTFSGISLSTGGVGVGWI | | TITCRASQSISNYLNWYQL | |
| | RQPPGKALEWLSLIYWND | | KPGNAPKLLIFAASSLQSG | |
| | DKRYSPSLKSRLTITKDSSK | | VPSRFSGSGSGTDFTLTISS | |
| | NQVVLTMTNMDPVDTATY | | LQPEDFATYFCQRSYSTLTF | |
| | YCAHSGLAAPFYNWFDPW | | GGGTKVEIK | |
| | GQGTLVTVSS | | | |
| 20B4 | QVQLVQSGAEVKKPGASV | 65 | DIVMTQSPDSLAVSLGERA | 76 |
| | KVSCKVSGYTLTEVFMHW | | TINCKSSQSVLYSSNNKNY | |
| | VRQAPGKGLEWMGGFDPE | | LAWYQQKPGQPPKLLIYW | |
| | DGETIYAQKFQGRVTMTE | | ASTRESGVPDRFSGSGSGT | |
| | DTFIDTAYMDLSSLRSEDT | | DFTLTISSLQAEDVAVYYC | |
| | AVYYCAIGWGSRYFDYWG | | HQYYSTPLTFGGGTKVEIK | |
| | QGTLVTVSS | | | |

### Example 4 Expression, Purification and Binding Characterization of Recombinant Antibody

### Molecular Cloning of Recombinant Antibodies

The cDNA sequences that encode VH and VL regions of selected clones were directly synthesized as DNA fragments with 5′-end in-frame leader sequence (MGWSCIILFLVATATGVHS). These DNA fragments were cloned into selected vectors using NEBuilder DNA Assembly Cloning Kit (New England Biolabs). VH region was cloned into pFUSE-CHIg_hGI vector (InvivoGen #pfuse-hchg1), which in-frame with constant region of hIgG1 heavy chain in the vector. VL region was cloned into pFUSE2-CLIg_hk vector (InvivoGen, 4pfuse2-hclk), which in-frame with constant region of hIg kappa light chain in the vector. The amino acid sequences of the constant region of hIgG1 heavy chain and the constant region of hIg kappa light chain are as follow:

>Heavy chain constant region (SEQ ID NO: 99):
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFSCSVMHEALHNHYTQ
KSLSLSPGK
>Light chain constant region (SEQ ID NO: 100):
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC

The IgG form of antibodies were disclosed as the following heavy chain and light chain full-lengths in Table 5. 1H10: SEQ ID NO: 77 (heavy chain) and 78 (light chain); 2F9: SEQ ID NO 79 (heavy chain) and 80 (light chain); 4F9: SEQ ID NO 81 (heavy chain) and 82 (light chain); 9E2: SEQ ID NO 83 (heavy chain) and 84 (light chain); 10E11: SEQ ID NO: 85 (heavy chain) and 86 (light chain); 13C7: SEQ ID NO 87 (heavy chain) and 88 (light chain); 14B12: SEQ ID NO 89 (heavy chain) and 90 (light chain); 15A4: SEQ ID NO 91 (heavy chain) and 92 (light chain); 17F3: SEQ ID NO 93 (heavy chain) and 94 (light chain); 19C7: SEQ ID NO: 95 (heavy chain) and 96 (light chain); 20B4: SEQ ID NO 97 (heavy chain) and 98 (light chain).

TABLE 5
Sequences of heavy chain and light chain full lengths for anti-CDH17 recombinant
antibodies

| | Sequence of Heavy Chain | | Sequence of Light Chain | |
|---|---|---|---|---|
| | | SEQ | | SEQ |
| Clone | Sequence | ID NO | Sequence | ID NO |
| 1H10 | QVQLVESGGGVVQPGRSLRL | 77 | DIQMTQSPSSVSASVGDRVTI | 78 |
| | SCVASGFTFSSYGMHWVRQA | | TCRASQDISSWLAWYQQQP | |
| | PGKGLEWVTFIWYDGSNKNY | | GKAPKLQIYAASSLQSGVPS | |
| | ADSVKGRFTISRDNSKNTLNL | | RFSGSGSGTDFTLTISSLQPE | |
| | QMNSLRVEDTAVYYCARDRY | | DFATYYCQQAKSFPPTFGQG | |
| | SSGWDVFDYWGQGTLVTVSS | | TKVEIKRTVAAPSVFIFPPSDE | |
| | ASTKGPSVFPLAPSSKSTSGG | | QLKSGTASVVCLLNNFYPRE | |
| | TAALGCLVKDYFPEPVTVSW | | AKVQWKVDNALQSGNSQES | |
| | NSGALTSGVHTFPAVLQSSGL | | VTEQDSKDSTYSLSSTLTLSK | |
| | YSLSSVVTVPSSSLGTQTYICN | | ADYEKHKVYACEVTHQGLS | |
| | VNHKPSNTKVDKKVEPKSCD | | SPVTKSFNRGEC | |
| | KTHTCPPCPAPELLGGPSVFLF | | | |
| | PPKPKDTLMISRTPEVTCVVV | | | |
| | DVSHEDPEVKFNWYVDGVE | | | |
| | VHNAKTKPREEQYNSTYRVV | | | |
| | SVLTVLHQDWLNGKEYKCK | | | |
| | VSNKALPAPIEKTISKAKGQP | | | |
| | REPQVYTLPPSREEMTKNQVS | | | |
| | LTCLVKGFYPSDIAVEWESNG | | | |
| | QPENNYKTTPPVLDSDGSFFL | | | |
| | YSKLTVDKSRWQQGNVFSCS | | | |
| | VMHEALHNHYTQKSLSLSPG | | | |
| | K | | | |
| 2F9 | EVQLVESGGGVVRPGGSLRLS | 79 | DIQMTQSPSSLSASIGDRVTIT | 80 |
| | CAASGFTFDDYGMSWVRQA | | CRASQGIRNALGWYQQKPG | |
| | PGKGLEWVSGLSWNGGNTY | | KAPKRLIYAASNLQSGVPSR | |
| | YADSVKGRFTISRDNAKNSLY | | FSGSGSGTEFTLTISSLQPEDF | |
| | LQMNSLRAEDTALYYCARGS | | ATYYCLQHNSYPFTFGPGTK | |
| | RYNWNYDAFDIWGQGTLVT | | VEIKRTVAAPSVFIFPPSDEQL | |
| | VSSASTKGPSVFPLAPSSKSTS | | KSGTASVVCLLNNFYPREAK | |
| | GGTAALGCLVKDYFPEPVTVS | | VQWKVDNALQSGNSQESVT | |
| | WNSGALTSGVHTFPAVLQSSG | | EQDSKDSTYSLSSTLTLSKAD | |
| | LYSLSSVVTVPSSSLGTQTYIC | | YEKHKVYACEVTHQGLSSPV | |
| | NVNHKPSNTKVDKKVEPKSC | | TKSFNRGEC | |
| | DKTHTCPPCPAPELLGGPSVF | | | |
| | LFPPKPKDTLMISRTPEVTCV | | | |
| | VVDVSHEDPEVKFNWYVDG | | | |
| | VEVHNAKTKPREEQYNSTYR | | | |
| | VVSVLTVLHQDWLNGKEYK | | | |
| | CKVSNKALPAPIEKTISKAKG | | | |
| | QPREPQVYTLPPSREEMTKNQ | | | |
| | VSLTCLVKGFYPSDIAVEWES | | | |
| | NGQPENNYKTTPPVLDSDGSF | | | |
| | FLYSKLTVDKSRWQQGNVFS | | | |
| | CSVMHEALHNHYTQKSLSLS | | | |
| | PGK | | | |
| 4F9 | QVQLQESGPGLVKPSGTLSLT | 81 | DIQMTQSPSSLSASVGDRVTI | 82 |
| | CAVSGGSISSSNWWSWVRQP | | TCRASQSISSYLNWYQQKPG | |
| | PGKGLEWIGEIYHSGSTSYNP | | KAPKLLIYAASSLQSGVPSRF | |
| | SLKSRVTISVDKSKNQFSLKL | | SGSGSGTHFTLTISSLQPEDF | |
| | SSVTAADTAVYYCARNDWGF | | ATYYCQQSHNIPITFGQGTRL | |
| | PLDCWGQGTLVTVSSASTKG | | EIKRTVAAPSVFIFPPSDEQLK | |
| | PSVFPLAPSSKSTSGGTAALG | | SGTASVVCLLNNFYPREAKV | |
| | CLVKDYFPEPVTVSWNSGAL | | QWKVDNALQSGNSQESVTE | |
| | TSGVHTFPAVLQSSGLYSLSSV | | QDSKDSTYSLSSTLTLSKAD | |
| | VTVPSSSLGTQTYICNVNHKP | | YEKHKVYACEVTHQGLSSPV | |
| | SNTKVDKKVEPKSCDKTHTC | | TKSFNRGEC | |
| | PPCPAPELLGGPSVFLFPPKPK | | | |
| | DTLMISRTPEVTCVVVDVSHE | | | |
| | DPEVKFNWYVDGVEVHNAK | | | |
| | TKPREEQYNSTYRVVSVLTVL | | | |
| | HQDWLNGKEYKCKVSNKAL | | | |
| | PAPIEKTISKAKGQPREPQVYT | | | |
| | LPPSREEMTKNQVSLTCLVKG | | | |
| | FYPSDIAVEWESNGQPENNYK | | | |
| | TTPPVLDSDGSFFLYSKLTVD | | | |
| | KSRWQQGNVFSCSVMHEAL | | | |
| | HNHYTQKSLSLSPGK | | | |
| 9E2 | EVQLVESGGGVVRPGGSLRLS | 83 | DIQMTQSPSSLSASVGDRVTI | 84 |
| | CAASGFTFDNYGMSWVRQA | | TCRASQGVRNALGWYQQKP | |
| | PGKGLEWVSGINWNGGSTYY | | GKAPKRLIYAASSLQSGVPS | |
| | ADSVRGRFTISRDNAKNSLYL | | RFSGSGSGTEFTLTISSLQPED | |
| | QMNSLRAEDTALYYCARGSR | | FATYYCLQHNSYPFTFGPGT | |
| | YNWSYDAFDIWGQGTLVTVS | | KVEIKRTVAAPSVFIFPPSDE | |
| | SASTKGPSVFPLAPSSKSTSGG | | QLKSGTASVVCLLNNFYPRE | |
| | TAALGCLVKDYFPEPVTVSW | | AKVQWKVDNALQSGNSQES | |
| | NSGALTSGVHTFPAVLQSSGL | | VTEQDSKDSTYSLSSTLTLSK | |
| | YSLSSVVTVPSSSLGTQTYICN | | ADYEKHKVYACEVTHQGLS | |
| | VNHKPSNTKVDKKVEPKSCD | | SPVTKSFNRGEC | |
| | KTHTCPPCPAPELLGGPSVFLF | | | |
| | PPKPKDTLMISRTPEVTCVVV | | | |
| | DVSHEDPEVKFNWYVDGVE | | | |
| | VHNAKTKPREEQYNSTYRVV | | | |
| | SVLTVLHQDWLNGKEYKCK | | | |
| | VSNKALPAPIEKTISKAKGQP | | | |
| | REPQVYTLPPSREEMTKNQVS | | | |
| | LTCLVKGFYPSDIAVEWESNG | | | |
| | QPENNYKTTPPVLDSDGSFFL | | | |
| | YSKLTVDKSRWQQGNVFSCS | | | |
| | VMHEALHNHYTQKSLSLSPG | | | |
| | K | | | |
| 10E11 | EVQLVQSGTEVKKPGESLKIS | 85 | DIQMTQSPSSVSASVGDRVTI | 86 |
| | CKDSGYRFSSYWIGWVRQMP | | TCRASQGISNWLGWYQQKP | |
| | GKGLEWMGIIYPGDSDTRYSP | | GKAPKLLIYAASSLQSGVPSR | |
| | SFQGQVTISADKSISTAYLQW | | FSGSGSGTDFTLTISSLQPEDF | |
| | RSLKASDTAMYYCARRGWV | | ATYYCQQANSFPLTFGGGTK | |
| | NYFDYWGQGTLVTVSSASTK | | VEIKRTVAAPSVFIFPPSDEQL | |
| | GPSVFPLAPSSKSTSGGTAAL | | KSGTASVVCLLNNFYPREAK | |
| | GCLVKDYFPEPVTVSWNSGA | | VQWKVDNALQSGNSQESVT | |
| | LTSGVHTFPAVLQSSGLYSLSS | | EQDSKDSTYSLSSTLTLSKAD | |
| | VVTVPSSSLGTQTYICNVNHK | | YEKHKVYACEVTHQGLSSPV | |
| | PSNTKVDKKVEPKSCDKTHT | | TKSFNRGEC | |
| | CPPCPAPELLGGPSVFLFPPKP | | | |
| | KDTLMISRTPEVTCVVVDVSH | | | |
| | EDPEVKFNWYVDGVEVHNA | | | |
| | KTKPREEQYNSTYRVVSVLTV | | | |
| | LHQDWLNGKEYKCKVSNKA | | | |
| | LPAPIEKTISKAKGQPREPQVY | | | |
| | TLPPSREEMTKNQVSLTCLVK | | | |
| | GFYPSDIAVEWESNGQPENNY | | | |
| | KTTPPVLDSDGSFFLYSKLTV | | | |
| | DKSRWQQGNVFSCSVMHEA | | | |
| | LHNHYTQKSLSLSPGK | | | |
| 13C7 | QITLKESGPTLVKPTQTLTLTC | 87 | DIQMTQSPSSLSASVGDRVTI | 88 |
| | TFSGFSLSTSGVGVGWIRQPP | | TCRASQSITSYLNWYQQKPG | |
| | GKALEWLALIYWNDDKRYRP | | KAPKLLIYAASSLQSGVPSRF | |
| | SLKNRLTITKDTSKNQVVLTM | | SGSGSGTDFTLTISSLQPEDF | |
| | TNMDPVDTATYYCTHYGVTA | | ATYYCQRSYSTLTFGGGTKV | |
| | PFYNWFDPWGQGTLVTVSSA | | EIKRTVAAPSVFIFPPSDEQLK | |
| | STKGPSVFPLAPSSKSTSGGTA | | SGTASVVCLLNNFYPREAKV | |
| | ALGCLVKDYFPEPVTVSWNS | | QWKVDNALQSGNSQESVTE | |
| | GALTSGVHTFPAVLQSSGLYS | | QDSKDSTYSLSSTLTLSKAD | |
| | LSSVVTVPSSSLGTQTYICNV | | YEKHKVYACEVTHQGLSSPV | |
| | NHKPSNTKVDKKVEPKSCDK | | TKSFNRGEC | |
| | THTCPPCPAPELLGGPSVFLFP | | | |
| | PKPKDTLMISRTPEVTCVVVD | | | |
| | VSHEDPEVKFNWYVDGVEV | | | |
| | HNAKTKPREEQYNSTYRVVS | | | |
| | VLTVLHQDWLNGKEYKCKV | | | |
| | SNKALPAPIEKTISKAKGQPRE | | | |
| | PQVYTLPPSREEMTKNQVSLT | | | |
| | CLVKGFYPSDIAVEWESNGQP | | | |
| | ENNYKTTPPVLDSDGSFFLYS | | | |
| | KLTVDKSRWQQGNVFSCSVM | | | |
| | HEALHNHYTQKSLSLSPGK | | | |
| 14B12 | QVQLQESGPGLVKPSETLSLT | 89 | DIQMTQSPSSLSASVGDRVTI | 90 |
| | CTVSGGSINTYYRSWIRQPPG | | TCRASQSITTYLNWYQQKPG | |
| | KGLEWIGYIYYSGNTNYNPSL | | KAPQLLIFAASRLQTGVPSRF | |
| | KSRVTISADTSKNQFSLNLISV | | SGSGSGTDFTLTISSLLPGDF | |
| | TAADTAMYYCARAQYGEEAF | | ATYYCQQSYNTPPTFGGGTK | |
| | DIWGQGTLVTVSSASTKGPSV | | VEIKRTVAAPSVFIFPPSDEQL | |
| | FPLAPSSKSTSGGTAALGCLV | | KSGTASVVCLLNNFYPREAK | |
| | KDYFPEPVTVSWNSGALTSG | | VQWKVDNALQSGNSQESVT | |
| | VHTFPAVLQSSGLYSLSSVVT | | EQDSKDSTYSLSSTLTLSKAD | |
| | VPSSSLGTQTYICNVNHKPSN | | YEKHKVYACEVTHQGLSSPV | |
| | TKVDKKVEPKSCDKTHTCPP | | TKSFNRGEC | |
| | CPAPELLGGPSVFLFPPKPKDT | | | |
| | LMISRTPEVTCVVVDVSHEDP | | | |
| | EVKFNWYVDGVEVHNAKTK | | | |
| | PREEQYNSTYRVVSVLTVLHQ | | | |
| | DWLNGKEYKCKVSNKALPAP | | | |
| | IEKTISKAKGQPREPQVYTLPP | | | |
| | SREEMTKNQVSLTCLVKGFYP | | | |
| | SDIAVEWESNGQPENNYKTTP | | | |
| | PVLDSDGSFFLYSKLTVDKSR | | | |
| | WQQGNVFSCSVMHEALHNH | | | |
| | YTQKSLSLSPGK | | | |
| 15A4 | QVQLVESGGGVVQPGRSLRL | 91 | DIQMTQSPSSVSASVGDRVTI | 92 |
| | SCAASGFNFSSYGMHWVRQA | | TCRASQGISSWLAWYQQKP | |
| | PGKGLEWVAFVWYDGSNQN | | GKAPKLLIFAASSLQSGVPSR | |
| | YADSVKGRFTISRDNSNNTLY | | FSGSGSGTGFTLTISSLQPEDF | |
| | LQMNSLRAEDTAVYYCARDR | | ATYYCQQAKSFPPTFGQGTK | |
| | YSSGWDVFDYWGQGTLVTV | | VEIKRTVAAPSVFIFPPSDEQL | |
| | SSASTKGPSVFPLAPSSKSTSG | | KSGTASVVCLLNNFYPREAK | |
| | GTAALGCLVKDYFPEPVTVS | | VQWKVDNALQSGNSQESVT | |
| | WNSGALTSGVHTFPAVLQSSG | | EQDSKDSTYSLSSTLTLSKAD | |
| | LYSLSSVVTVPSSSLGTQTYIC | | YEKHKVYACEVTHQGLSSPV | |
| | NVNHKPSNTKVDKKVEPKSC | | TKSFNRGEC | |
| | DKTHTCPPCPAPELLGGPSVF | | | |
| | LFPPKPKDTLMISRTPEVTCV | | | |
| | VVDVSHEDPEVKFNWYVDG | | | |
| | VEVHNAKTKPREEQYNSTYR | | | |
| | VVSVLTVLHQDWLNGKEYK | | | |
| | CKVSNKALPAPIEKTISKAKG | | | |
| | QPREPQVYTLPPSREEMTKNQ | | | |
| | VSLTCLVKGFYPSDIAVEWES | | | |
| | NGQPENNYKTTPPVLDSDGSF | | | |
| | FLYSKLTVDKSRWQQGNVFS | | | |
| | CSVMHEALHNHYTQKSLSLS | | | |
| | PGK | | | |
| 17F3 | EVQLVESGGGLVKSGGSLRLS | 93 | DIQMTQSPSSVSASIGDRVTI | 94 |
| | CAASGFTFSSYSLKWVRQAP | | TCRASQDISTWFAWYQQKP | |
| | GKGLEWVSSISSSSGYIYYAD | | GKAPKLLIYAASTLRSGVPSR | |
| | SVKGRFTISRDNAKNSLYLQM | | FSGSGSGTDFTLTINSLQPED | |
| | NSLRAEDTAMYYCARAVSTI | | FATYYCQQANSFPLTFGGGT | |
| | AARPGYYYYYMDVWGKGTL | | KVEIKRTVAAPSVFIFPPSDE | |
| | VTVSSASTKGPSVFPLAPSSKS | | QLKSGTASVVCLLNNFYPRE | |
| | TSGGTAALGCLVKDYFPEPVT | | AKVQWKVDNALQSGNSQES | |
| | VSWNSGALTSGVHTFPAVLQS | | VTEQDSKDSTYSLSSTLTLSK | |
| | SGLYSLSSVVTVPSSSLGTQT | | ADYEKHKVYACEVTHQGLS | |
| | YICNVNHKPSNTKVDKKVEP | | SPVTKSFNRGEC | |
| | KSCDKTHTCPPCPAPELLGGP | | | |
| | SVFLFPPKPKDTLMISRTPEVT | | | |
| | CVVVDVSHEDPEVKFNWYV | | | |
| | DGVEVHNAKTKPREEQYNST | | | |
| | YRVVSVLTVLHQDWLNGKEY | | | |
| | KCKVSNKALPAPIEKTISKAK | | | |
| | GQPREPQVYTLPPSREEMTKN | | | |
| | QVSLTCLVKGFYPSDIAVEWE | | | |
| | SNGQPENNYKTTPPVLDSDGS | | | |
| | FFLYSKLTVDKSRWQQGNVF | | | |
| | SCSVMHEALHNHYTQKSLSL | | | |
| | SPGK | | | |
| 19C7 | QITLKESGPTLVKPTQTLTLTC | 95 | DIQMTQSPSSLSASVGDRVTI | 96 |
| | TFSGISLSTGGVGVGWIRQPP | | TCRASQSISNYLNWYQLKPG | |
| | GKALEWLSLIYWNDDKRYSP | | NAPKLLIFAASSLQSGVPSRF | |
| | SLKSRLTITKDSSKNQVVLTM | | SGSGSGTDFTLTISSLQPEDF | |
| | TNMDPVDTATYYCAHSGLAA | | ATYFCQRSYSTLTFGGGTKV | |
| | PFYNWFDPWGQGTLVTVSSA | | EIKRTVAAPSVFIFPPSDEQLK | |
| | STKGPSVFPLAPSSKSTSGGTA | | SGTASVVCLLNNFYPREAKV | |
| | ALGCLVKDYFPEPVTVSWNS | | QWKVDNALQSGNSQESVTE | |
| | GALTSGVHTFPAVLQSSGLYS | | QDSKDSTYSLSSTLTLSKAD | |
| | LSSVVTVPSSSLGTQTYICNV | | YEKHKVYACEVTHQGLSSPV | |
| | NHKPSNTKVDKKVEPKSCDK | | TKSFNRGEC | |
| | THTCPPCPAPELLGGPSVFLFP | | | |
| | PKPKDTLMISRTPEVTCVVVD | | | |
| | VSHEDPEVKFNWYVDGVEV | | | |
| | HNAKTKPREEQYNSTYRVVS | | | |
| | VLTVLHQDWLNGKEYKCKV | | | |
| | SNKALPAPIEKTISKAKGQPRE | | | |
| | PQVYTLPPSREEMTKNQVSLT | | | |
| | CLVKGFYPSDIAVEWESNGQP | | | |
| | ENNYKTTPPVLDSDGSFFLYS | | | |
| | KLTVDKSRWQQGNVFSCSVM | | | |
| | HEALHNHYTQKSLSLSPGK | | | |
| 20B4 | QVQLVQSGAEVKKPGASVKV | 97 | DIVMTQSPDSLAVSLGERATI | 98 |
| | SCKVSGYTLTEVFMHWVRQA | | NCKSSQSVLYSSNNKNYLA | |
| | PGKGLEWMGGFDPEDGETIY | | WYQQKPGQPPKLLIYWASTR | |
| | AQKFQGRVTMTEDTFIDTAY | | ESGVPDRFSGSGSGTDFTLTI | |
| | MDLSSLRSEDTAVYYCAIGW | | SSLQAEDVAVYYCHQYYSTP | |
| | GSRYFDYWGQGTLVTVSSAS | | LTFGGGTKVEIKRTVAAPSVF | |
| | TKGPSVFPLAPSSKSTSGGTA | | IFPPSDEQLKSGTASVVCLLN | |
| | ALGCLVKDYFPEPVTVSWNS | | NFYPREAKVQWKVDNALQS | |
| | GALTSGVHTFPAVLQSSGLYS | | GNSQESVTEQDSKDSTYSLS | |
| | LSSVVTVPSSSLGTQTYICNV | | STLTLSKADYEKHKVYACEV | |
| | NHKPSNTKVDKKVEPKSCDK | | THQGLSSPVTKSFNRGEC | |
| | THTCPPCPAPELLGGPSVFLFP | | | |
| | PKPKDTLMISRTPEVTCVVVD | | | |
| | VSHEDPEVKFNWYVDGVEV | | | |
| | HNAKTKPREEQYNSTYRVVS | | | |
| | VLTVLHQDWLNGKEYKCKV | | | |
| | SNKALPAPIEKTISKAKGQPRE | | | |
| | PQVYTLPPSREEMTKNQVSLT | | | |
| | CLVKGFYPSDIAVEWESNGQP | | | |
| | ENNYKTTPPVLDSDGSFFLYS | | | |
| | KLTVDKSRWQQGNVFSCSVM | | | |
| | HEALHNHYTQKSLSLSPGK | | | |

### Expression and Purification of Recombinant Antibodies

The heavy chain expression plasmid and light chain plasmids were co-transfected into CHO cells (ATCC, Cat #CCL-61) using ExpiFectamine 293 Transfection Kit (ThermoFisher, A14524), or into ExpiCHO-S cells (ThermoFisher #A29127) using ExpiFectamine CHO Transfection Kit (ThermoFisher, A29129). Based on the manufacturer's instructions, plasmid DNA concentration reached 1.0 μg per ml of suspended cells, with LC:HC vector ratio 1:1. The transfected cells were cultured 5 to 7 days on an orbital shaker at 37 C, 8% CO₂. Conditioned medium was collected and antibodies were purified using HiTrap MabSelect SuRe column (Cytiva, #17549112) on AKTA Pure 25 machine (Cytiva). Eluted antibodies were neutralized with Tris Buffer (pH 9.0) and subjected to PBS buffer exchange. Product concentration was measured by UV absorption, and quality was determined by SDS-PAGE and HPLC.

### Binding Characterization of Anti-CDH17 Recombinant Antibody to CDH17 Positive and CDH17 Negative Cell Lines by Flow Cytometry

Binding of the recombinant antibody (human IgG1) to the cell surface CDH17 was determined by FACS analysis using cancer cell lines including CDH17 positive cancer cell AsPC1 cells and CDH17 negative cancer cell SW480 cells.

AsPC1 cells were maintained in RPMI-1640 medium supplemented with 10% FBS and 1% penicillin and streptomycin. SW480 cells were maintained in DMEM medium supplemented with 10% FBS and 1% penicillin and streptomycin. Cells were cultured at 37° C. with 5% CO₂ in humidified atmosphere.

To determine the binding of recombinant antibody to cell surface CDH17 receptors, cells were first harvested and resuspended in cell staining buffer (BioLegend, Cat #420201) at 1.3-1.5×10⁶ cells/mL. Then, the cells were treated with human Fc receptor blocking reagent (BioLegend, Cat #422302) on the ice for 10 min. The resulting cell suspension was aliquoted into 50 μL aliquots. 25 μL of recombinant antibody at various concentrations were mixed with the cell aliquots, the final concentration of recombinant antibody in the mixture ranged from 1.1 pM to 200 nM. The cells were incubated on the ice for 1 hour and then washed with cell staining buffer twice. 50 μL of secondary antibody (PE conjugated goat anti-human Fc, eBioscience™, 1:250 dilution) was added to each sample to resuspend the cells. The cells were incubated on the ice for another 20 min. Cells were subsequently washed twice with cell staining buffer and resuspended in 4% PFA to fix the cells. The samples were analyzed using iQue3 to measure the median fluorescence intensity using corresponding channels.

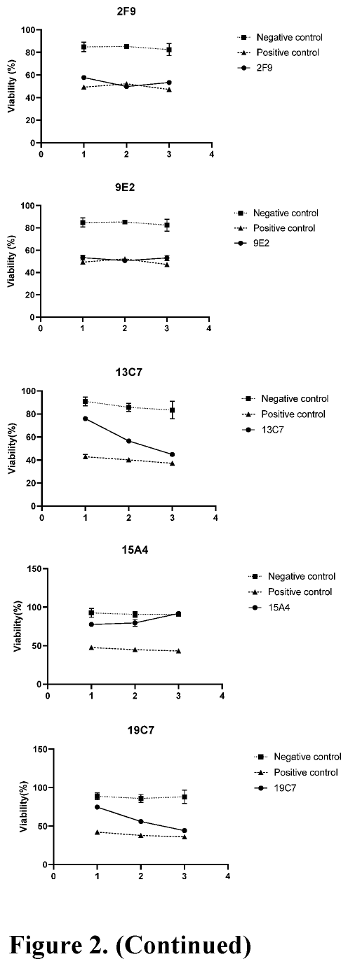

HBMAB-81 was a reformatted mAb with the CDH17 binding arm from bispecific antibody BI 905771 and human IgG1 backbone, (See WO2018115231A2; SEQ ID:116, SEQ ID:117). B12 is an in-house isotype control antibody disclosed in patent US005652138A. The result was disclosed in FIG. 3 and Table 6. As a result, it was confirmed that the anti-CDH17 antibodies of the present disclosure specifically bind to the human CDH17 as originally expressed in cells in a concentration-dependent pattern, while HBMAB81 non-specifically binds to CDH17-negative cell line SW480.

TABLE 6
K_{D} values of anti-CDH17 recombinant antibodies
binding to CDH17 positive AsPC1 cells

| Antibody | K_{D} (nM) |
|---|---|
| 1H10 | 0.31 |
| 2F9 | 0.17 |
| 4F9 | 1.69 |
| 9E2 | 0.31 |
| 10E11 | 0.10 |
| 13C7 | 0.28 |
| 14B12 | 0.07 |
| 15A4 | 2.32 |
| 17F3 | 0.74 |
| 19C7 | 0.13 |
| 20B4 | 0.10 |

### Example 5 Characterization of Anti-CDH17 Recombinant Antibody Cell Internalization in CDH17 Expression Cells by Indirect Killing Assay

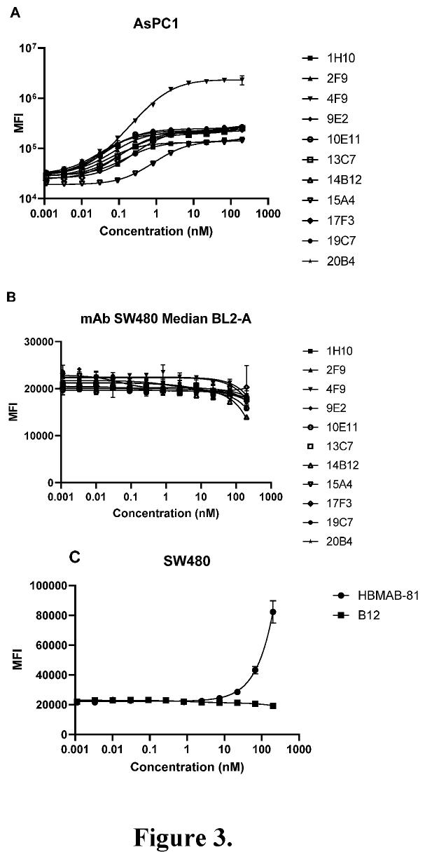

To evaluate the endocytosis of the antibodies induced by anti-CDH17 antibodies binding, an indirect killing assay was used to characterize the antibody internalization. Fab anti-human IgG Fc-MMAF conjugates with cleavable linker (Moradec, AH-202AF) was incubated and complexed with the recombinant anti-CDH17 antibodies. The formed complexes were then incubated with CDH17-expressing cells. Upon binding to the cell surface CDH17 receptors, the complexes were internalized and release the conjugated MMAF after lysosomal cleavage of the linker. The released MMAF subsequently inhibited the cell division by blocking the polymerization of tubulin. Briefly, a CDH17 expressing cell line AsPC1 cells were seeded in 96-well plate at 5,000 cells/well and incubated overnight. The recombinant anti-CDH17 antibodies were mixed with the Fab anti-human IgG Fc-MMAF conjugates with cleavable linker at 1:6 ratio (mol/mol) and incubated for 10 min to form the complex. Then, a series dilution of the complexes, from 4.5 pM to 30 nM, were added to each well and the cell viability was measured by Cytation5 imaging system for 48 hours. The dose-response curves were plotted and fitted by GraphPad Prism 9. As shown in FIG. 4 and Table 7, compared to the negative control clone (B12), all the antibody clones showed killing effect in AsPC1 cell line indicating MMAF was delivered into the cells efficiently.

TABLE 7
IC₅₀ of indirect killing assay to evaluate the internalization
of recombinant anti-CDH17 antibodies in AsPC1 cells.

| Clone ID | IC₅₀ (nM) |
|---|---|
| 1H10 | 0.19 |
| 2F9 | 0.14 |
| 4F9 | 0.29 |
| 9E2 | 0.15 |
| 10E11 | 0.08 |
| 13C7 | 0.09 |
| 14B12 | 0.09 |
| 15A4 | 1.99 |
| 17F3 | 0.08 |
| 19C7 | 0.09 |
| 20B4 | 0.07 |

### Example 6 Binding Activities of Anti-CDH17 Clones to Cross-Species CDH17 and Homologs by Octet Characterization

The affinity between CDH17 antibodies and CDH17 antigens and homologues was determined by Octet (Octet Red 384) instrument. Anti-hIgG Fc Capture (AHC) Biosensors were selected, and the sensors were balanced with buffer solution for 10 min. Subsequently, sensors were dipped into the wells containing 1 μg/ml of CDH17 lead antibodies to load onto the probes. The excess unbound antibodies were washed off. The antigen binding was performed in the wells containing serially diluted human CDH17-His tag (Sino biological, catalog number 11360-H08H), cynomolgus CDH17-His tag (Acro Biosystems, catalog number CA7-C52H4), homologs human CDH6-His tag (Acro Biosystems, catalog number CA6-H5229) and human CDH16-His tag (Sino biological, catalog number 10915-H08H), with concentration ranging from 200 nM to 3.1 nM, for 5 min. Then probes were dipped into wells containing renewed buffer to initiate the dissociation for another 15 min.

The results as shown in Table 8. The 10E11, 14B12 and 20B4 bound to both human and cynomolgus CDH17 proteins, while HBMAB81 only bound to human CDH17 protein.

TABLE 8
Binding activities of anti-CDH17 clones
to cross-species CDH17 and homologs.

| | | Antigen proteins | | | |
|---|---|---|---|---|---|
| Clone ID | | hCDH17 | cCDH17 | hCDH6 | hCDH16 |
| HBMAB81 | K_{D} (nM) | 4.0 | N/A | N/A | N/A |
| | Kₒₙ (1/Ms) | 3.4E+05 | N/A | N/A | N/A |
| | K_{off} (1/s) | 1.36E−03 | N/A | N/A | N/A |
| 10E11 | K_{D} (nM) | 14.2 | 15.0 | N/A | N/A |
| | Kₒₙ (1/Ms) | 9.75E+04 | 8.00E+04 | N/A | N/A |
| | K_{off} (1/s) | 1.38E−03 | 1.20E−03 | N/A | N/A |
| 14B12 | K_{D} (nM) | 2.2 | 2.5 | N/A | N/A |
| | Kₒₙ (1/Ms) | 2.3E+05 | 6.2E+05 | N/A | N/A |
| | K_{off} (1/s) | 5.07E−04 | 1.6E−04 | N/A | N/A |
| 20B4 | K_{D} (nM) | 1.1 | 3.0 | N/A | N/A |
| | Kₒₙ (1/Ms) | 1.62E+05 | 9.80E+04 | N/A | N/A |
| | K_{off} (1/s) | 1.87E−04 | 2.94E−04 | N/A | N/A |

Note:
N/A: no binding

### Example 7 Binding Competition

20B4 and HBMAB81 mAbs were labeled with Alexa Flour 647 according to the manufacturer's instruction (ThermoFisher #A20186). In the binding competition assay, 20B4-AF647 and HBMAB81-AF647 were fixed at their EC80 concentration 1.51 nM and 0.97 nM, respectively. The 20B4 and HBMAB81 ranging from 0.03 nM to 1800 nM were first mixed with 1.51 nM 20B4-AF647 or 0.97 nM HBMAB81-647, then incubated with AsPC1 cells on ice for 1 h. Cells were washed twice with cell staining buffer. The samples were analyzed using iQue3 to measure the median fluorescence intensity using corresponding channels.

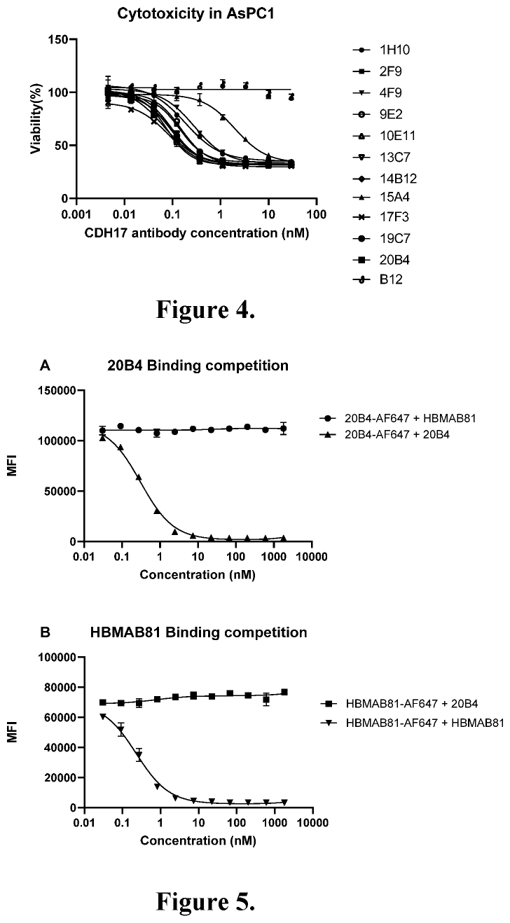

The result was disclosed in FIG. 5. The groups “20B4-AF647+HBMAB81” and “HBMAB-AF647+20B4” didn't show binding inhibition. This indicated that 20B4 and HBMAB81 do not bind to the same or similar epitope.

### Example 8 Generation of Anti-CDH17 Antibody Drug Conjugates (ADCs

The antibodies of the present invention have cell affinity activity and endocytosis activity, making them suitable for coupling with drugs to form antibody-drug conjugates for treating CDH17-mediated diseases.

### Purification of the Monoclonal Antibody

Referring to the preparation procedures of anti-CDH17 antibody described in Example 4. Mota, 1H10, 2F9, 4F9, 9E2, 10E11, 13C7, 14B12, 17F3, 19C7, 20B4 and HBMAB-81 are purified for drug conjugation. Mota is an in-house isotype antibody obtained from patent US008568726B2. Further antibodies for conjugation may include any antibodies described herein (see Example 4).

### Preparation of Intermediates as Drugs

The following intermediate compound was used for the generation of antibody-drug conjugates (ADCs) for anti-CDH17 antibodies, me-vc-MMAE drug linker may be generated similar to the procedure described in US 2005/0238649, Compound C was prepared by a method disclosed in PCT Patent Application. (See WO2020063673, filed Sep. 25, 2019), Compound D was prepared by a method disclosed in PCT Patent Application. (See WO2022161385, filed Jan. 26, 2022).

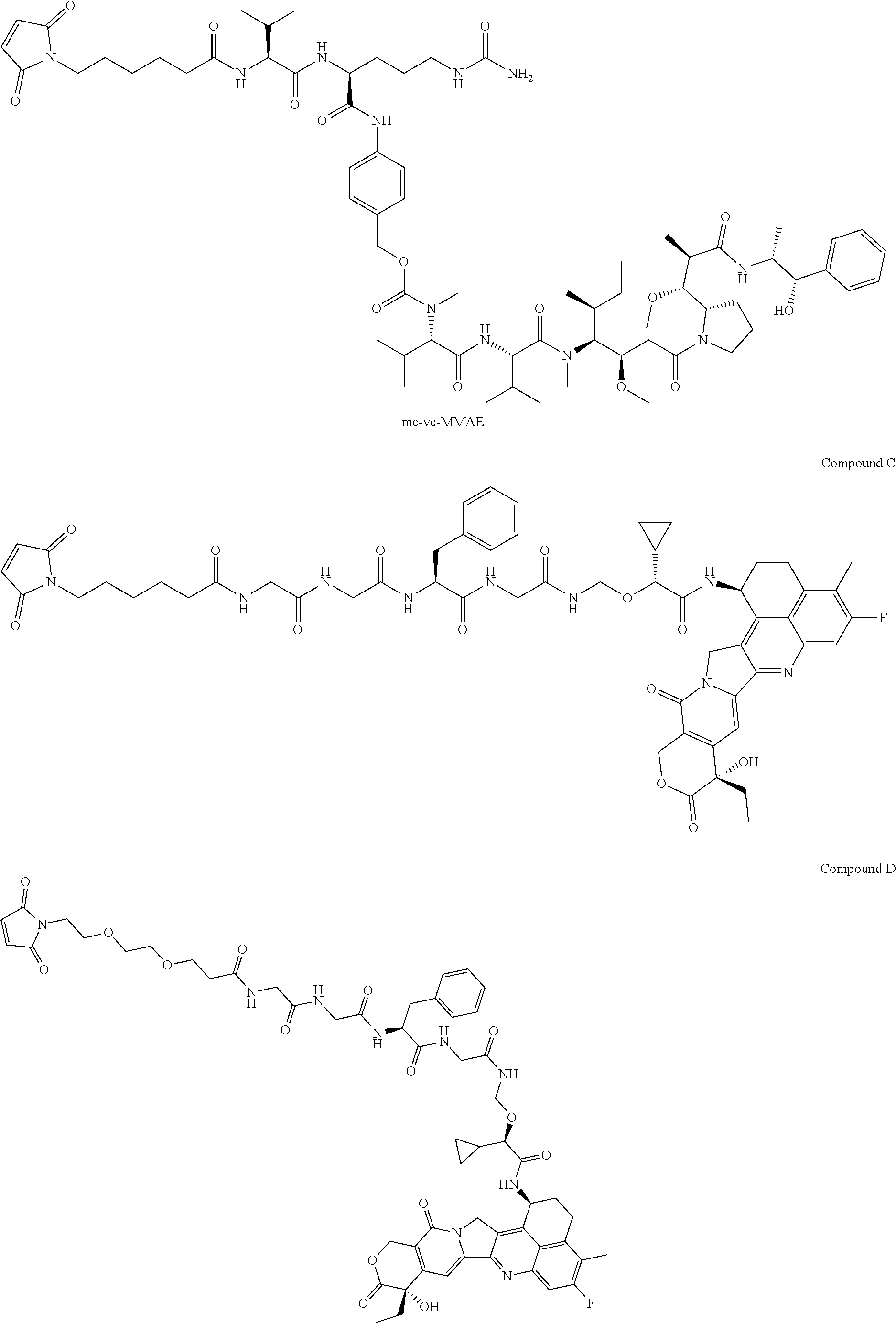

Conjugation of Monoclonal Antibody with Drug Molecule

### Compound D

Anti-CDH17 antibody (5 mg/mL in PBS, pH 7.4) was treated with 10 mM of (tris(2-carboxylethyl)phosphine (TCEP) in excess equivalence at 37° C. for 2 hours. Sufficient molar equivalence (6-12 eq) of drug linker (Compound D, in DMSO) was added to the reduced antibody in PBS. The samples were then incubated overnight with Compound D at room temperature. The drug-to-antibody ratio (DAR) of the ADCs was determined using a 6230 LC/MS-TOF system (Agilent) and the average values were summarized in Table 9.

### Compound C

Anti-CDH17 antibody (5 mg/mL in PBS, pH 7.4) was treated with 10 mM of (tris(2-carboxylethyl)phosphine (TCEP) in excess equivalence at 37° C. for 2 hours. Sufficient molar equivalence (6-12 eq) of drug linker (Compound C, in DMSO) was added to the reduced antibody in PBS. The samples were then incubated overnight with Compound C at 4° C. with rotation. The drug-to-antibody ratio (DAR) of the ADCs was determined using a 6230 LC/MS-TOF system (Agilent) and the average values were summarized in Table 9.

### Vc-MMAE

Anti-CDH17 antibody (5 mg/mL in PBS, pH 7.4) was treated with 10 mM of (tris(2-carboxylethyl)phosphine (TCEP) in excess equivalence at 37° C. for 1 hour. Sufficient molar equivalence (8 eq) of drug linker (vc-MMAE in DMSO) was added to the reduced antibody in PBS. The samples were then incubated for 1 hour with vc-MMAE at room temperature. The drug-to-antibody ratio (DAR) of the ADCs was determined using a 6230 LC/MS-TOF system (Agilent) and the average values were summarized in Table 9. The average DAR values of the anti-CDH17 ADCs conjugated to vc-MMAE were approximately 4.0.

TABLE 9
Conjugation of monoclonal antibody and linker/payload moiety

| | Monoclonal | Linker/Payload | Average |
|---|---|---|---|
| Compound No | Antibody | Moiety | DAR |
| ADC-01 | 1H10 | Compound D | 8.0 |
| ADC-02 | 2F9 | Compound D | 8.0 |
| ADC-03 | 4F9 | Compound D | 8.0 |
| ADC-04 | 9E2 | Compound D | 8.0 |
| ADC-05 | 10E11 | Compound D | 8.0 |
| ADC-06 | 13C7 | Compound D | 8.0 |
| ADC-07 | 14B12 | Compound D | 8.0 |
| ADC-08 | 17F3 | Compound D | 8.0 |
| ADC-09 | 19C7 | Compound D | 8.0 |
| ADC-10 | 20B4 | Compound D | 8.0 |
| ADC-11 | HBMAB-81 | Compound D | 8.0 |
| ADC-12 | B12 | Compound D | 8.0 |
| ADC-13 | 10E11 | Compound D | 4.5 |
| ADC-14 | 10E11 | Compound D | 6.0 |
| ADC-15 | 14B12 | Compound D | 4.0 |
| ADC-16 | 14B12 | Compound D | 6.2 |
| ADC-18 | 20B4 | Compound D | 5.6 |
| ADC-21 | 20B4 | Compound C | 4.3 |
| ADC-23 | 20B4 | Compound C | 8.0 |
| ADC-24 | HBMAB-81 | Compound C | 3.7 |
| ADC-27 | Mota | Compound C | 6.3 |
| ADC-28 | 10E11 | mc-vc-MMAE | 4.5 |
| ADC-29 | 14B12 | mc-vc-MMAE | 4.4 |
| ADC-30 | 20B4 | mc-vc-MMAE | 4.2 |
| ADC-32 | B12 | mc-vc-MMAE | 3.8 |
| ADC-34 | 20B4 | Compound C | 6.4 |
| ADC-35 | 20B4 | Compound C | 6.0 |
| ADC-36 | 20B4 | Compound C | 3.9 |

### Example 9 Inhibitory Effect of the ADCs on Growth of Tumor Cells

### In Vitro Cytotoxicity of ADCs in Multiple Cell Lines

The ADC immunoconjugates, which had different antibodies targeting CDH17 with different internalization, were analyzed for this Example. CDH17-mediated cell toxicities by these conjugates were tested in cell culture to define the potency of various linker-cytotoxic agent combinations. Different types of cancer cell lines were tested, including colorectal cancer cell line GP2d, NCI-1H716, SK-CO-1, SNU16, ESO26 and SW480; pancreatic cancer cell line AsPC1.

We used two methods (Cell titer glo and Cytation5) to test the cytotoxicity of immunoconjugates in the above-listed cell lines. For the Cytation5 method, cells were collected in log phase growth and distributed into 96-well plates at 5000 cells/well with propidium iodide (abeam, Cat #ab14083) at 500 ng/mL and SPY650-DNA (Cytoskeleton, Inc., Cat #CYSC501) at 1:2000 dilution. Cells were incubated overnight at 37° C. in 5% CO₂. The immunoconjugates were diluted with cell culture medium containing 500 ng/mL propidium iodide and 2000-fold diluted SPY650-DNA, then added to each well. The final concentrations of the immunoconjugates were ranging from 0.003 nM to 200 nM. Cytation5 (Agilent) was used to detect the viability of the cells in each well by imaging the plates every 12 h for 96 h. For Cell titer glo method. The cells were seeded in 96 well plates at 2000 cells/well. After overnight incubation, the immunoconjugates were added to each well. The final concentrations of the immunoconjugates in the wells were ranging from 0.0001 nM to 200 nM. After 5 or 7 days of incubation, the cell viability in each well was determined by Cell Titer Glo 2.0 Assay (Promega).

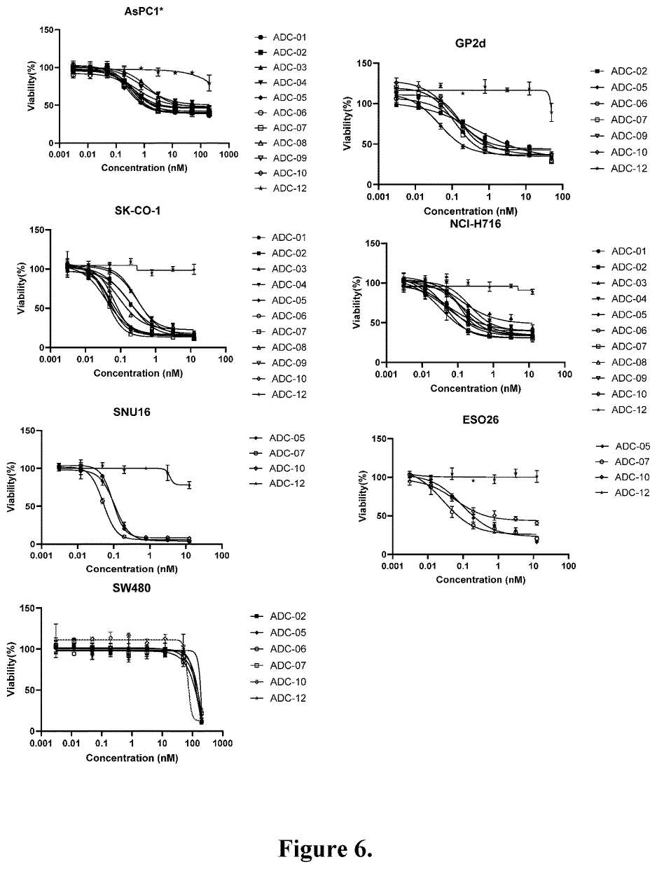

Curves and IC₅₀ values were generated in GraphPad Prism using a sigmoidal dose-response non-linear regression fit. The data from these experiments are summarized in Table 10 to 11 and FIG. 6. All the ADC molecules showed good potential of cytotoxicity on tumor cells. The ADC molecules showed CDH17 expression level dependent cytotoxicity: the higher the CDH expression level, the more toxic the ADC is to cells, which indicates that the ADC molecule is less toxic to cells that do not express CDH17 and may be safer in vivo. The anti-CDH17 antibodies conjugated with mc-vc-MMAE also have excellent tumor cell cytotoxicity.

TABLE 10
Evaluation of the in vitro cytotoxicity of
antibody-drug conjugates on tumor cells

| | Cell Line | | | | | | |
|---|---|---|---|---|---|---|---|
| | | | NCI- | SK- | | | |
| Compound | AsPC1* | GP2d | H716 | CO-1 | SNU16 | ESO26 | SW480 |
| No | IC₅₀ values (nM) | | | | | | |
| ADC-01 | 0.52 | 0.49 | 0.14 | 0.20 | — | — | — |
| ADC-02 | 0.42 | 0.47 | 0.13 | 0.16 | — | — | >100 |
| ADC-03 | 1.32 | — | 0.11 | 0.30 | — | — | — |
| ADC-04 | 0.85 | — | 0.22 | 0.27 | — | — | — |
| ADC-05 | 0.25 | 0.04 | 0.03 | 0.05 | 0.11 | 0.10 | >100 |
| ADC-06 | 0.37 | 0.20 | 0.05 | 0.05 | — | — | >100 |
| ADC-07 | 0.32 | 0.12 | 0.05 | 0.04 | 0.05 | 0.03 | >100 |
| ADC-08 | 0.45 | 0.20 | 0.03 | 0.09 | — | — | — |
| ADC-09 | 0.46 | 0.11 | 0.12 | 0.04 | — | — | — |
| ADC-10 | 0.40 | 0.13 | 0.07 | 0.07 | 0.10 | 0.06 | >100 |
| ADC-12 | NA | NA | NA | NA | NA | NA | NA |

Note1:
*Cytotoxicity assay using Cytation5 (96 h). The others are using Celltiter Glo assay (7 days)
Note2:
NA: not applicable or IC₅₀ is not conclusive based on current dose-response curve (indicating no/low toxicity)
Note3:
“—”: not tested

TABLE 11
Evaluation of the in vitro cytotoxicity of
antibody-drug conjugates on tumor cells

| | Cell Line | | | | | |
|---|---|---|---|---|---|---|
| Compound | AsPC1* | GP2d | SNU5 | OE19^{#} | SNU16 | ESO26 |
| No | IC₅₀ values (nM) | | | | | |
| ADC-28 | 0.09 | 0.47 | 0.09 | 0.20 | 0.02 | 0.01 |
| ADC-29 | 0.05 | 0.23 | 0.03 | 0.05 | 0.02 | 0.01 |
| ADC-30 | 0.10 | 0.25 | 0.05 | 0.12 | 0.03 | 0.02 |
| ADC-32 | NA | NA | NA | NA | NA | NA |

Note1:
*Cytotoxicity assay using Cytation5 (96 h). The others are using Celltiter Glo assay for 5 days or
^{#}7 days.
Note2:
NA: not applicable or IC50 is not conclusive based on current dose-response curve (indicating no/low toxicity)

In Vitro Cytotoxicity Comparison of Anti-CDH17 ADCs with Different DAR Values

The same methods (Cell titer glo and Cytation5) were used to test the cytotoxicity of anti-CDH17 ADCs with different DAR values. Cytation5 (Agilent) was used to detect the viability of the AsPC1 cells in each well by imaging the plates every 12 h for 96 h. The final concentrations of the immunoconjugates were ranging from 0.003 nM to 200 nM. Cell Titer Glo 2.0 Assay (Promega) was used to detect cell viability in GP2d and SKCO1 cell lines after 7 days of incubation. The final concentrations of the immunoconjugates in the wells ranged from 0.0007 nM to 50 nM.

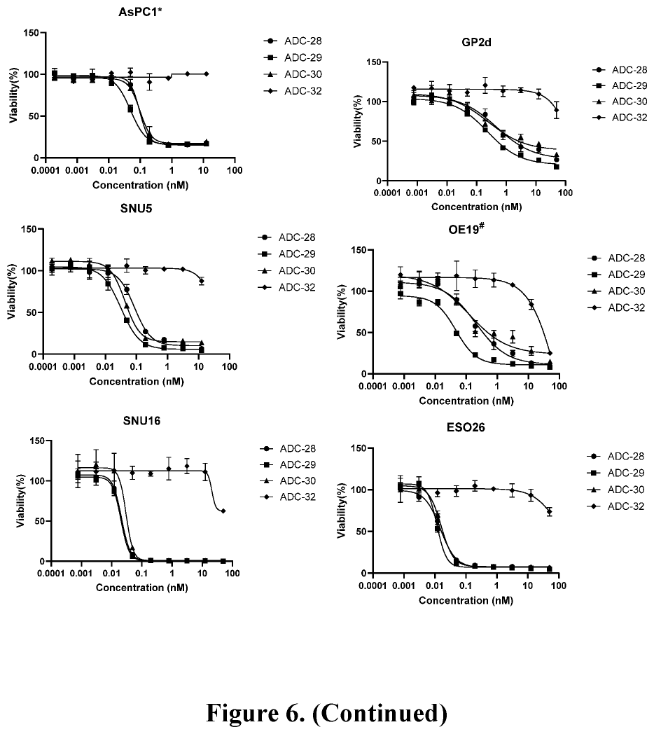

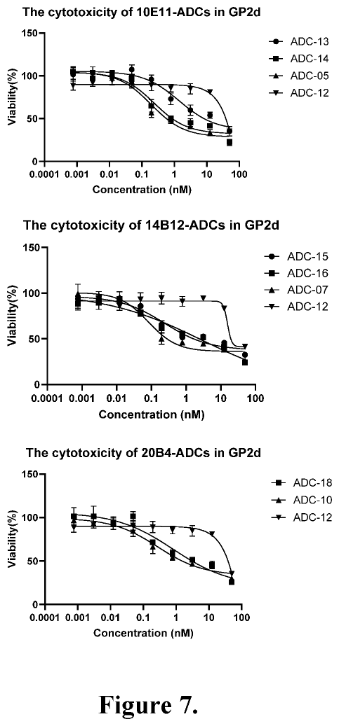

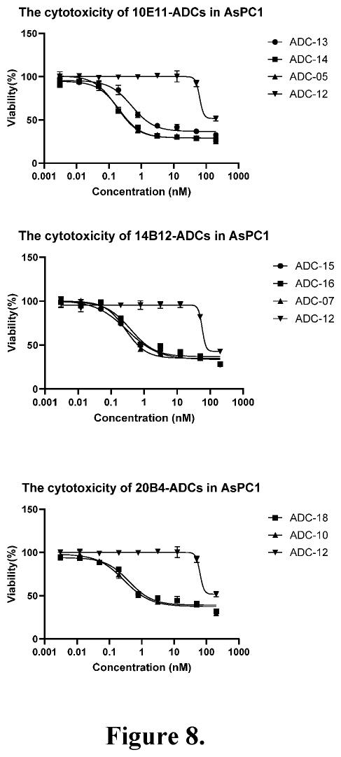

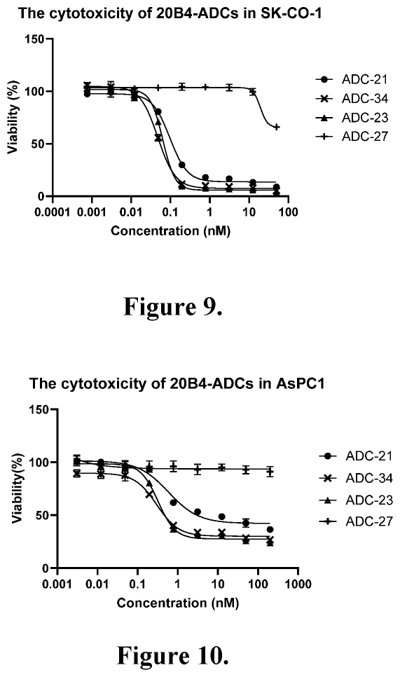

Curves and IC₅₀ values were generated in GraphPad Prism using a sigmoidal dose-response non-linear regression fit. The data of anti-CDH17 ADCs with compound D are summarized in Table 12, FIG. 7 to 8. All the ADCs with different DAR values showed cytotoxicity on tumor cells. The data of anti-CDH17 ADCs with compound C are summarized in Table 13, FIG. 9 to 10. The cytotoxicity of all the ADCs is in the reasonable range.

TABLE 12
Comparison of the in vitro cytotoxicity of
antibody-drug conjugates on tumor cells

Cell Line

| | | | |
|---|---|---|---|
| Compound | Antibody | AsPC1* | GP2d |

| | | | |
|---|---|---|---|
| No | clone | DAR | IC₅₀ values (nM) |

| | | | | |
|---|---|---|---|---|
| ADC-13 | 10E11 | 4.5 | 0.50 | 1.40 |
| ADC-14 | 10E11 | 6.0 | 0.21 | 0.24 |
| ADC-05 | 10E11 | 8.0 | 0.17 | 0.19 |
| ADC-15 | 14B12 | 4.0 | 0.30 | 0.14 |
| ADC-16 | 14B12 | 6.2 | 0.38 | 2.97 |
| ADC-07 | 14B12 | 8.0 | 0.28 | 0.08 |
| ADC-18 | 20B4 | 5.6 | 0.40 | 0.83 |
| ADC-10 | 20B4 | 8.0 | 0.28 | 0.28 |
| ADC-12 | B12 | 8.0 | NA | NA |

Note:
*Cytotoxicity assay using Cytation5 (96 h). The others are using Celltiter Glo assay (7 days)

TABLE 13
comparison of the in vitro cytotoxicity of
antibody-drug conjugates on tumor cells

Cell Line

| | | | |
|---|---|---|---|
| Compound | Antibody | SK-CO-1 | AsPC1* |

| | | | |
|---|---|---|---|
| No | clone | DAR | IC₅₀ values (nM) |

| | | | | |
|---|---|---|---|---|
| ADC-21 | 20B4 | 4.3 | 0.10 | 0.59 |
| ADC-34 | 20B4 | 6.4 | 0.05 | 0.30 |
| ADC-23 | 20B4 | 8.0 | 0.06 | 0.32 |
| ADC-27 | Mota | 6.3 | NA | NA |

*Cytotoxicity assay using Cytation5 (96 h). The others are using Celltiter Glo assay (7 days).

### Example 10 Tumor-Inhibitory Experiment of ADCs Towards CDH17 Positive Cancer Cell Nude Mouse Subcutaneous Transplantation Tumor Model

### Efficacy of Anti-CDH17 ADCs in a Human Pancreatic Cancer Cell Xenograft Mouse Model

AsPC1 cells (pancreatic cancer) were engrafted subcutaneously into female BALB/c nude mice. When the tumor volume reached approximately 150-200 mm³, the engrafted mice were randomized into seven groups (5 mice per group). The groups were vehicle control, ADC-02, ADC-05, ADC-06, ADC-07, ADC-10, and ADC-11. The mice were treated with ADCs (6 mg/kg) intravenously Q4D×3. Mean tumor growth inhibition (TGI) was calculated utilizing the following formula:

TGI=((mean(C)−mean(C₀))−(mean(T)−mean(T₀)))/(mean(C)−mean(C₀))*100%; T is current group value, C is control group value, T₀ and C₀ represent the tumor volume at the beginning of the test.

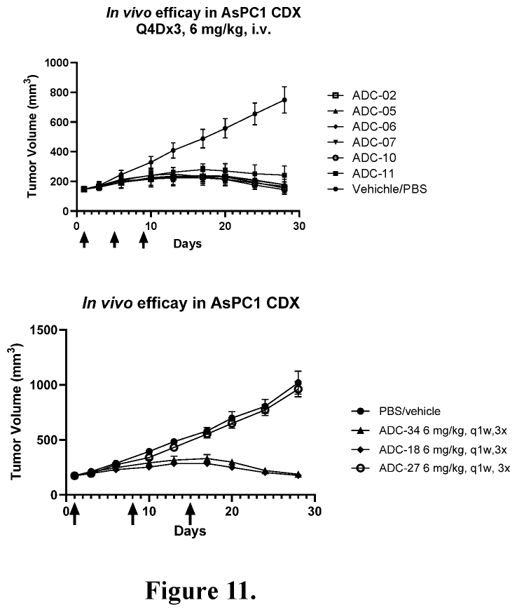

The results of the study were shown in Table 14 and FIG. 11. Tumor growth in mice treated with ADC-02, ADC-05, ADC-06, ADC-07, and ADC-10 was inhibited compared to the vehicle/PBS group. ADC-10 has the highest tumor inhibition rate (80.76%), followed by ADC-02 (79.25%), ADC-07 (77.77%), ADC-05 (76.27%), and ADC-06 (76.26%), which are all better than the positive control ADC-11 (67.62%). The anti-CDH17 antibodies conjugated with compound C also have excellent tumor inhibition (Table 15 and FIG. 11).

TABLE 14
Efficacy of ADCs in AsPC1 tumor-bearing mice

| | Test | | Mice | Tumor volume (mm3) | TGI |
|---|---|---|---|---|---|
| Groups | Articles | Dose | No. | Day 28 | Day 28 |
| 1 | Vehicle/ | 10 μL/g | 5 | 748.7 ± 39.8 | — |
| | PBS | Q4D × 3 | | | |
| 2 | ADC-02 | 6 mg/kg | | 155.4 ± 18.6 | 79.25% |
| 3 | ADC-05 | Q4D × 3 | | 177.6 ± 24.5 | 76.27% |
| 4 | ADC-06 | | | 177.8 ± 16.1 | 76.26% |
| 5 | ADC-07 | | | 166.5 ± 13.2 | 77.77% |
| 6 | ADC-10 | | | 144.1 ± 9.2 | 80.76% |
| 7 | ADC-11 | | | 242.5 ± 27.6 | 67.62% |

TABLE 15
Efficacy of ADCs in AsPC1 tumor-bearing mice

| | | | | Tumor volume | |
|---|---|---|---|---|---|
| | Test | | Mice | (mm3) | TGI |
| Groups | Articles | Dose | No. | Day 28 | Day 28 |
| 1 | Vehicle/ | 10 μL/g, | 5 | 1019.0 ± 105.6 | — |
| | PBS | Q1W × 3 | | | |
| 2 | ADC-34 | 6 mg/kg, | | 186.7 ± 21.5 | 98.42% |
| 3 | ADC-18 | Q1W × 3 | | 176.4 ± 9.0 | 99.66% |
| 4 | ADC-27 | | | 960.1 ± 68.7 | 6.91% |

### Efficacy of Anti-CDH17 ADCs in a Human Colorectal Cancer Cell Xenograft Mouse Model

GP2d cells (colorectal cancer) were engrafted subcutaneously into female BALB/c nude mice. When the tumor volume reached approximately 100-150 mm³, the engrafted mice were randomized into seven groups (5 mice per group). The groups were vehicle control, ADC-02, ADC-05, ADC-06, ADC-07, ADC-10, and ADC-11. The mice were treated with ADCs (6 mg/kg) intravenously Q4D×3.

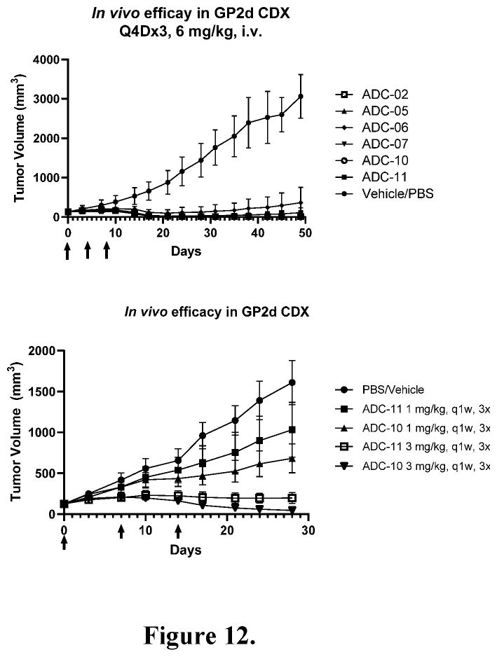

The results of the study were shown in Table 16 to 17 and FIG. 12. In terms of the in vivo efficacy, compared to PBS in the control group, all the ADC molecules have effects in inhibiting the increase of tumors in volume. Tumor growth in mice treated with ADC-02, ADC-05, ADC-06, ADC-07, and ADC-10 was inhibited compared to the vehicle/PBS group, with TGI values of 104.20%, 104.22%, 92.03%, 104.22%, and 104.10%, respectively. Among them, the tumor growth of ADC-02, ADC-05, ADC-07, and ADC-10 was inhibited compared to the ADC-11 control group. The tumor volumes in ADC-11 group increased after around 30 days of 1^{st} treatment. Our ADC showed better efficacy compared to the positive control ADC-11.

The anti-CDH17 antibodies conjugated with compound C also have an excellent tumor inhibition effect (Table 18, FIG. 12). ADCs conjugated to compound C with different DAR values have been compared in GP2d CDX model. Tumor inhibition has been observed in all the treatment groups. The ADCs showed comparable in vivo efficacy when they had equal equivalence of payload (Table 19, FIG. 12).

TABLE 16
Efficacy of ADCs in GP2d tumor-bearing mice

| | | | | Tumor volume | |
|---|---|---|---|---|---|
| | Test | | Mice | (mm3) | TGI |
| Groups | Articles | Dose | No. | Day 49 | Day 49 |
| 1 | Vehicle/ | 10 μL/g | 5 | 3065.0 ± 247.0 | — |
| | PBS | Q4D × 3 | | | |
| 2 | ADC-02 | 6 mg/kg | | 7.0 ± 1.0 | 104.20% |
| 3 | ADC-05 | Q4D × 3 | | 6.0 ± 0.0 | 104.22% |
| 4 | ADC-06 | | | 364.0 ± 175.0 | 92.03% |
| 5 | ADC-07 | | | 6.0 ± 1.0 | 104.22% |
| 6 | ADC-10 | | | 7.0 ± 1.0 | 104.18% |
| 7 | ADC-11 | | | 112.0 ± 53.0 | 100.61% |

TABLE 17
Efficacy comparison of anti-CDH17 ADCs vs.
positive control in GP2d tumor-bearing mice

| | | | | Tumor volume | |
|---|---|---|---|---|---|
| | Test | | Mice | (mm3) | TGI |
| Groups | Articles | Dose | No. | Day 28 | Day 28 |
| 1 | Vehicle/ | 10 μL/g, | 5 | 1611.0 ± 270.0 | — |
| | PBS | Q1W × 3 | | | |
| 2 | ADC-11 | 1 mg/kg, | | 1033.0 ± 336.0 | 38.94% |
| | | Q1W × 3 | | | |
| 3 | ADC-10 | 1 mg/kg, | | 683.0 ± 177.0 | 62.52% |
| | | Q1W × 3 | | | |
| 4 | ADC-11 | 3 mg/kg, | | 197.0 ± 66.0 | 95.22% |
| | | Q1W × 3 | | | |
| 5 | ADC-10 | 3 mg/kg, | | 45.0 ± 9.0 | 105.50% |
| | | Q1W × 3 | | | |

TABLE 18
Efficacy of ADCs in GP2d tumor-bearing mice

| | | | | Tumor volume | |
|---|---|---|---|---|---|
| | Test | | Mice | (mm3) | TGI |
| Groups | Articles | Dose | No. | Day 28 | Day 28 |
| 1 | Vehicle/ | 10 μL/g, | 5 | 2389.0 ± 210.0 | — |
| | PBS | Q1W × 3 | | | |
| 2 | ADC-34 | 3 mg/kg, | | 85.0 ± 33.0 | 102.33% |
| | | Q1W × 3 | | | |
| 3 | ADC-18 | 3 mg/kg, | | 12.0 ± 4.0 | 105.53% |
| | | Q1W × 3 | | | |
| 4 | ADC-27 | 3 mg/kg, | | 1538.0 ± 166.0 | 37.79% |
| | | Q1W × 3 | | | |

TABLE 19
Efficacy comparison of anti-CDH17 ADCs with different
DAR values in GP2d tumor-bearing mice

| | | | | Tumor volume | |
|---|---|---|---|---|---|
| | Test | | Mice | (mm3) | TGI |
| Groups | Articles | Dose | No. | Day 21 | Day 21 |
| 1 | Vehicle/ | 10 μL/g, single | 6 | 1574 ± 253 | |
| | PBS | dose | | | |
| 2 | ADC-35 | 6 mg/kg, single | | 74 ± 19 | 104.27% |
| | | dose | | | |
| 3 | ADC-35 | 4 mg/kg, single | | 192 ± 70 | 96.03% |
| | | dose | | | |
| 4 | ADC-35 | 2 mg/kg, single | | 809 ± 145 | 53.20% |
| | | dose | | | |
| 5 | ADC-36 | 9 mg/kg, single | | 23 ± 1 | 107.82% |
| | | dose | | | |
| 6 | ADC-36 | 6 mg/kg, single | | 201 ± 113 | 95.42% |
| | | dose | | | |
| 7 | ADC-36 | 3 mg/kg, single | | 562 ± 99 | 70.33% |
| | | dose | | | |
| 8 | ADC-36 | 2 mg/kg, single | | 612 ± 76 | 66.85% |
| | | dose | | | |

### Efficacy of Anti-CDH17 ADCs in a Human Gastric Cancer Cell Xenograft Mouse Model

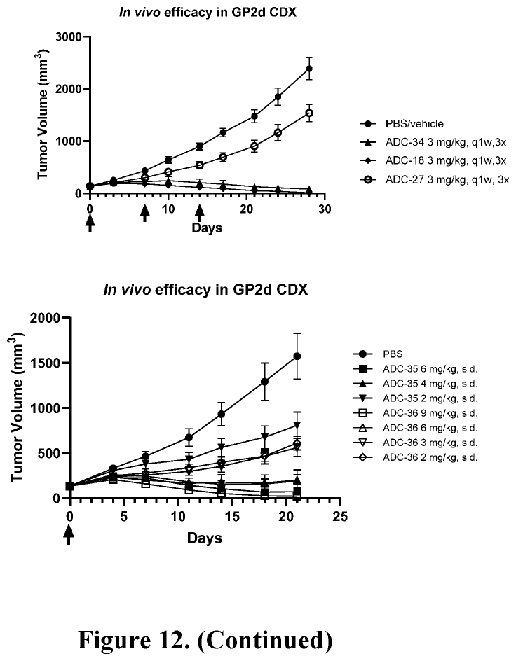

SNU16 cells (gastric cancer) were engrafted subcutaneously into female NU/J mice. When the tumor volume reached approximately 150-200 mm³, the engrafted mice were randomized into ten groups (5 mice per group). The detailed dosing strategy is described in Table 20. ADCs conjugated to compound C with different DAR values. In ADC-24 3 mg/kg group, the tumor started to regrow after 17 days of treatment (FIG. 13). There is a significant difference in tumor volume between ADC-24 and ADC-21 at lower dose.

TABLE 20
Efficacy comparison of anti-CDH17 ADCs with different
DAR values in SNU16 tumor-bearing mice

| | | | | Tumor volume | |
|---|---|---|---|---|---|
| | Test | | Mice | (mm3) | TGI |
| Groups | Articles | Dose | No. | Day 38 | Day 31 |
| 1 | Vehicle/ | 10 μL/g, single | 5 | 1062 ± 296 | / |
| | PBS | dose | | (31 day) | |
| 2 | ADC-34 | 6 mg/kg, single | | 26 ± 4 | 113.64% |
| | | dose | | | |
| 3 | ADC-34 | 4 mg/kg, single | | 131 ± 21 | 107.04% |
| | | dose | | | |
| 4 | ADC-34 | 2 mg/kg, single | | 196 ± 45 | 103.40% |
| | | dose | | | |
| 5 | ADC-21 | 9 mg/kg, single | | 158 ± 21 | 106.05% |
| | | dose | | | |
| 6 | ADC-21 | 6 mg/kg, single | | 252 ± 41 | 99.79% |
| | | dose | | | |
| 7 | ADC-21 | 3 mg/kg, single | | 187 ± 48 | 100.77% |
| | | dose | | | |
| 8 | ADC-21 | 2 mg/kg, single | | 153 ± 32 | 107.18% |
| | | dose | | | |
| 9 | ADC-24 | 6 mg/kg, single | | 291 ± 40 | 94.35% |
| | | dose | | | |
| 10 | ADC-24 | 3 mg/kg, single | | 369 ± 50 | 87.48% |
| | | dose | | | |

### Example 11 Pharmacokinetic Test of the ADCs

To study the pharmacokinetic of the ADCs, ADC-02, ADC-05, ADC-06, ADC-07, ADC-10, and ADC-11 were injected in Balb/c mice (3 mice per group) at a concentration of 5 mg/kg by i.v. After the injection, at designated time points (pre-bleed (blank), 1 h, 4 h, 24 h, 2 day, 3 day, 5 day, 7 day, 14 day, 21 day), the mouse serum samples were collected, and stored in −80° C.

### Detecting the Total Antibodies in Mouse Serum

Human CDH17 His Tag protein (Sino biological, catalog number 11360-H08H) was coated onto the MSD bare standard plates (Meso Scale Discovery, catalog number L15XA-3) at a concentration of 4 μg/mL overnight in 4° C. The excess unbound proteins were washed off by PBST buffer and were blocked with 3% BSA in PBS buffer for 1 h at room temperature with a shaking speed of 700 rpm. After washing with PBST for 3 times, standard samples, QC samples, SC samples, and mouse serum samples were added to plates for 1 h incubation at room temperature with a shaking speed of 700 rpm. The final concentrations of the standard samples were ranging from 0.02 ng/mL to 1000 ng/mL. Quality control (QC) samples and sample control (SC) samples were diluted into 4 different concentrations (0.06 ng/ml, 0.244 ng/ml, 3.906 ng/mL, and 250 ng/mL). Mouse serum samples were also diluted to the detection range. The plates were then washed with PBST for 3 times and incubated with sulfo-tag conjugated anti-Fc antibody for 1 h at room temperature with shaking. Finally, 150 μL MSD GOLD Read Buffer B was added to each well and the plate was read by MESO QuickPlex SQ 120 MM (Meso Scale Discovery). The raw data was analyzed on MSD discovery workbench software and the AUC data of the total antibody from mouse serum was calculated by pK solver.

### Detecting the ADCs in Mouse Serum

Human CDH17 His Tag protein (Sino biological, catalog number 11360-H08H) was coated onto the MSD bare standard plates (Meso Scale Discovery, catalog number L15XA-3) at a concentration of 8 μg/mL overnight in 4° C. The excess unbound proteins were washed off by PBST buffer and were blocked with 3% BSA in PBS buffer for 1 h at room temperature with a shaking speed of 700 rpm. After washing with PBST for 3 times, standard samples, QC samples, SC samples, and mouse serum samples were added to plates for 1 h incubation at room temperature with a shaking speed of 700 rpm. The final concentrations of the standard samples were ranging from 0.02 ng/mL to 1000 ng/mL. Quality control (QC) samples and sample control (SC) samples were diluted into 4 different concentrations (0.06 ng/ml, 0.244 ng/ml, 3.906 ng/mL, and 250 ng/mL). Mouse serum samples were also diluted to the detection range. The plates were then washed with PBST for 3 times and incubated with sulfo-tag conjugated anti-compound D antibody for 1 h at room temperature with shaking. Finally, 150 μL MSD GOLD Read Buffer B was added to each well and the plate was read by MESO QuickPlex SQ 120 MM (Meso Scale Discovery). The raw data was analyzed on MSD discovery workbench software and the AUC data of the ADC from mouse serum was calculated by pK solver.

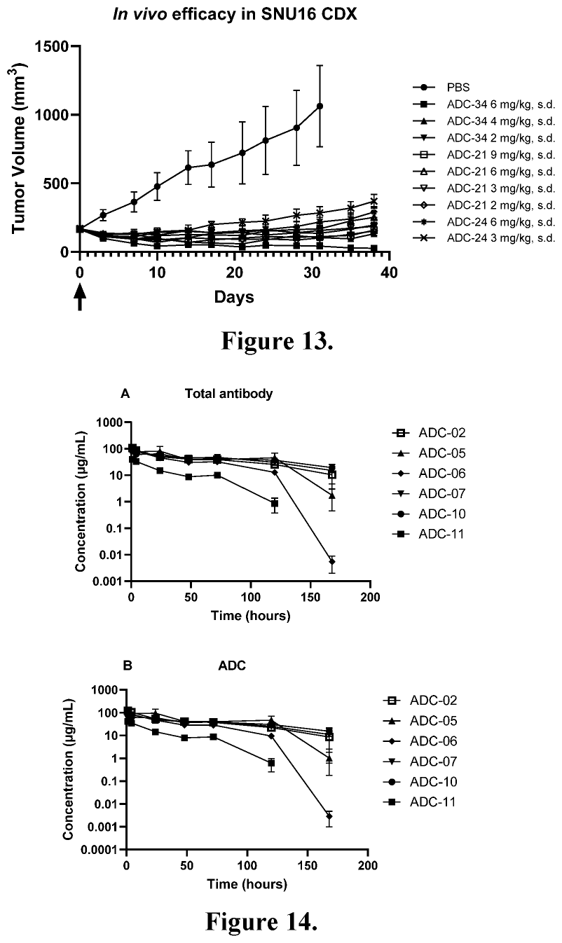

The total antibody here is referring to the antibody from ADC, we test both total antibody and ADCs when doing the assay. The results of the study were shown in FIG. 14 and Table 21 to 22. All the ADC molecules have good pharmacokinetic properties. The AUCs of ADC-02, ADC-05, ADC-06, ADC-07, and ADC-10 were significantly greater than the control group ADC-11. The ADC-02, ADC-05, ADC-06, ADC-07, and ADC-10 could be detected in the 7-day mouse serum samples, while ADC-11 could only be detected in the 5-day mouse serum sample.

TABLE 21
In vivo Pharmacokinetics (PK) of total antibodies after single dose administration to mice

| | HBMAB81 | 10E11 | 14B12 | 13C7 | 20B4 | 2F9 |
|---|---|---|---|---|---|---|
| Parameter | (ADC-11) | (ADC-05) | (ADC-07) | (ADC-06) | (ADC-10) | (ADC-02) |
| Cmax (μg/ml) | 40.0 | 101.8 | 104.0 | 103.1 | 81.8 | 106.3 |
| AUC 0-inf_obs | 1431.6 | 13133.2 | 10227.0 | 5211.4 | 11928.6 | 9215.3 |
| (μg/ml*h) | | | | | | |
| MRT 0-t (h) | 34.2 | 53.7 | 50.3 | 41.7 | 52.7 | 47.1 |
| Cl_obs | 0.0035 | 0.0004 | 0.0005 | 0.0010 | 0.0004 | 0.0005 |
| ((mg/kg)/(μg/ml)/h) | | | | | | |
| Vss_obs | 0.13 | 0.07 | 0.07 | 0.06 | 0.07 | 0.08 |
| ((mg/kg)/(μg/ml)) | | | | | | |

TABLE 22
In vivo Pharmacokinetics (PK) of conjugated antibodies
after single dose administration to mice

| Parameter | ADC-11 | ADC-05 | ADC-07 | ADC-06 | ADC-10 | ADC-02 |
|---|---|---|---|---|---|---|
| Cmax (μg/ml) | 41.4 | 120.6 | 108.1 | 112.5 | 82.1 | 120.4 |
| AUC 0-inf_obs | 1360.3 | 12230.3 | 9136.5 | 4722.5 | 9150.4 | 8085.2 |
| (μg/ml*h) | | | | | | |
| MRT 0-t (h) | 31.8 | 51.1 | 47.9 | 38.4 | 50.4 | 43.5 |
| Cl_obs | 0.0037 | 0.0004 | 0.0005 | 0.0011 | 0.0005 | 0.0006 |
| ((mg/kg)/(μg/ml)/h) | | | | | | |
| Vss_obs | 0.12 | 0.06 | 0.08 | 0.06 | 0.07 | 0.06 |
| ((mg/kg)/(μg/ml)) | | | | | | |

Similar administration, detection, and analysis methods were applied to ADC-21, ADC-24 and ADC-34. Balb/c mice (3 mice per group) were dosed at a concentration of 5 mg/kg by i.v. injection. Serum samples were collected at 13 time points: pre-bleed, 1 h, 4 h, 24 h, 2 day, 3 day, 5 day, 7 day, 9 day, 12 day, 14 day, 17 day and 21 day. MSD bare standard plates were coated at a concentration of 2 μg/mL for both total antibody and ADC PK detection. Quality control (QC) samples and sample control (SC) samples were diluted into 3 different concentrations (0.22 ng/ml, 3.52 ng/ml, and 225 ng/mL).

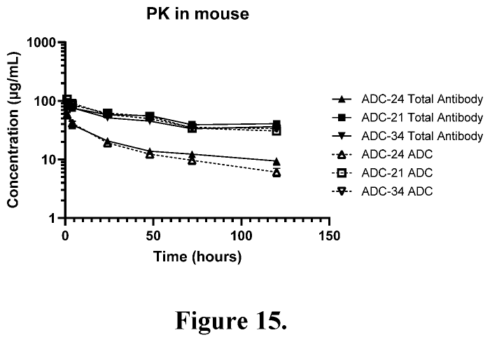

Sulfo-tag conjugated anti-Fc antibody and anti-payload antibody were used to detect the total antibodies and ADCs respectively. Table 23 to 24 showed that the AUCs of ADC-21 and ADC-34 were greater compared to ADC-24 (FIG. 15).

TABLE 23
In vivo Pharmacokinetics (PK) of total antibodies
after single dose administration to mice

| | HBMAB81 | 20B4 | 20B4 |
|---|---|---|---|
| Parameter | (ADC-24) | (ADC-21) | (ADC-34) |
| Cmax (μg/ml) | 57.65 | 91.61 | 76.17 |
| AUC 0-inf_obs | 3800.75 | 12357.43 | 11260.56 |
| (μg/ml*h) | | | |
| MRT 0-t (h) | 42.75 | 52.02 | 51.25 |
| Cl_obs | 0.001 | 0.0004 | 0.0004 |
| ((mg/kg)/(μg/ml)/h) | | | |
| Vss_obs | 0.21 | 0.07 | 0.08 |
| ((mg/kg)/(μg/ml)) | | | |

TABLE 24
In vivo Pharmacokinetics (PK) of conjugated antibodies
after single dose administration to mice

| Parameter | ADC-24 | ADC-21 | ADC-34 |
|---|---|---|---|
| Cmax (μg/ml) | 57.80 | 106.10 | 90.72 |
| AUC 0-inf_obs (μg/ml*h) | 2462.77 | 9320.07 | 9905.63 |
| MRT 0-t (h) | 37.50 | 47.05 | 48.37 |
| Cl_obs | 0.002 | 0.0005 | 0.0005 |
| ((mg/kg)/(μg/ml)/h) | | | |
| Vss_obs | 0.17 | 0.06 | 0.06 |
| ((mg/kg)/(μg/ml)) | | | |

Although specific embodiments of the invention have been described in detail, those skilled in the art will understand that various modifications and changes may be made to the details in accordance with all the published teachings, which are also included in the scope of the invention. The protection scope of the invention is defined by the appended claims and any equivalents thereof.

1. An antibody-drug conjugate of general formula (I), or a pharmaceutically acceptable salt or solvate thereof, 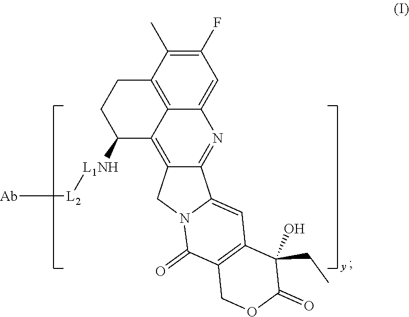
wherein:
L₁ and L₂ are linking units; wherein the L₂ is -La-Lb-Lc-Ld-,
the La is as shown in general formula (II-b): 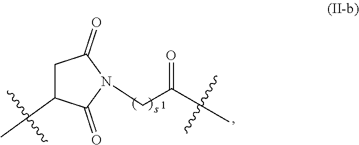
s1 is selected from 4, 5, 6, 7 or 8;
the Lb is a chemical bond;
the Lc is a tetrapeptide residue; preferably, Lc is a tetrapeptide residue of glycine-glycine-phenylalanine-glycine (GGFG);
the Ld is —NR1(CR2R3)s3-, wherein R1, R2 and R3 are identical or different and are each independently hydrogen or alkyl, and s3 is 1 or 2; wherein the La terminus is connected to Ab, and the Ld terminus is connected to L1;
y is a number selected from 1 to 10, preferably a number selected from 2 to 8, and most preferably 2, 4, 6, 8;
Ab is the anti-CDH17 antibody or antigen-binding fragment thereof, wherein the anti-CDH17 antibody or the antigen-binding fragment thereof comprises a heavy chain variable region and a light chain variable region of the antibody, wherein:
a heavy chain variable region comprises HCDR1, HCDR2 and HCDR3 having the amino acid sequences as shown in SEQ ID NO: 10, SEQ ID NO:21 and SEQ ID NO: 31, respectively; and a light chain variable region comprises LCDR1, LCDR2 and LCDR3 having the amino acid sequences as shown in SEQ ID NO: 42, SEQ ID NO: 47 and SEQ ID NO: 54, respectively; or
a heavy chain variable region comprises HCDR1, HCDR2 and HCDR3 having the amino acid sequences as shown in SEQ ID NO: 07, SEQ ID NO:17 and SEQ ID NO: 28, respectively; and a light chain variable region comprises LCDR1, LCDR2 and LCDR3 having the amino acid sequences as shown in SEQ ID NO: 38, SEQ ID NO: 45 and SEQ ID NO: 53, respectively; or
a heavy chain variable region comprises HCDR1, HCDR2 and HCDR3 having the amino acid sequences as shown in SEQ ID NO: 05, SEQ ID NO: 15 and SEQ ID NO: 26, respectively; and a light chain variable region comprises LCDR1, LCDR2 and LCDR3 having the amino acid sequences as shown in SEQ ID NO: 36, SEQ ID NO: 43 and SEQ ID NO: 51, respectively.

2. The antibody-drug conjugate or the pharmaceutically acceptable salt or solvate thereof according to claim 1, wherein the anti-CDH17 antibody or antigen-binding fragment thereof is a monoclonal antibody or antigen-binding fragment thereof, a polyclonal antibody or antigen-binding fragment thereof, a multi-specific antibody or antigen-binding fragment thereof, a murine antibody or antigen-binding fragment thereof, a chimeric antibody or antigen-binding fragment thereof, a humanized antibody or antigen-binding fragment thereof, a recombinant antibody or antigen-binding fragment thereof, a human antibody or antigen-binding fragment thereof; preferably, the multi-specific antibody is a bispecific antibody, a tri-specific antibody or a tetra-specific antibody.

3. The antibody-drug conjugate or the pharmaceutically acceptable salt or solvate thereof according to claim 1, wherein the anti-CDH17 antibody or antigen-binding fragment thereof comprises: a heavy chain variable region comprising an amino acid sequence selected from: SEQ ID NOs: 65, 61, 59, or sequence having at least 80%, 85%, 90%, 95% or 99% sequence identity therewith; and/or a light chain variable region comprising an amino acid sequence selected from: SEQ ID NOs: 76, 72, 70, or sequence having at least 80%, 85%, 90%, 95% or 99% sequence identity therewith.

4. The antibody-drug conjugate or the pharmaceutically acceptable salt or solvate thereof according to claim 1, wherein the anti-CDH17 antibody or antigen-binding fragment thereof comprising:
the heavy chain variable region having the amino acid sequence as shown in SEQ ID NO: 65, or having the amino acid sequence of at least 80%, 85%, 90%, 95% or 99% sequence identity therewith; and/or the light chain variable region having the amino acid sequence as shown in SEQ ID NO: 76, or having the amino acid sequence of at least 80%, 85%, 90%, 95% or 99% sequence identity therewith;
or
the heavy chain variable region having the amino acid sequence as shown in SEQ ID NO: 61, or having the amino acid sequence of at least 80%, 85%, 90%, 95% or 99% sequence identity therewith; and/or the light chain variable region having the amino acid sequence as shown in SEQ ID NO: 72, or having the amino acid sequence of at least 80%, 85%, 90%, 95% or 99% sequence identity therewith;
or
the heavy chain variable region having the amino acid sequence as shown in SEQ ID NO: 59, or having the amino acid sequence of at least 80%, 85%, 90%, 95% or 99% sequence identity therewith; and/or the light chain variable region having the amino acid sequence as shown in SEQ ID NO: 70, or having the amino acid sequence of at least 80%, 85%, 90%, 95% or 99% sequence identity therewith.

5. The antibody-drug conjugate or the pharmaceutically acceptable salt or solvate thereof according to claim 1, wherein the anti-CDH17 antibody or antigen-binding fragment thereof comprising:
the heavy chain variable region having the amino acid sequence as shown in SEQ ID NO: 65; and/or the light chain variable region having the amino acid sequence as shown in SEQ ID NO: 76;
or
the heavy chain variable region having the amino acid sequence as shown in SEQ ID NO: 61; and/or the light chain variable region having the amino acid sequence as shown in SEQ ID NO: 72;
or
the heavy chain variable region having the amino acid sequence as shown in SEQ ID NO: 59; and/or the light chain variable region having the amino acid sequence as shown in SEQ ID NO: 70.

6. The antibody-drug conjugate or the pharmaceutically acceptable salt or solvate thereof according to claim 1, wherein the anti-CDH17 antibody or antigen-binding fragment thereof comprising:
the heavy chain variable region having the amino acid sequence as shown in SEQ ID NO: 65 and the light chain variable region having the amino acid sequence as shown in SEQ ID NO: 76;
or
the heavy chain variable region having the amino acid sequence as shown in SEQ ID NO: 61 and the light chain variable region having the amino acid sequence as shown in SEQ ID NO: 72;
or
the heavy chain variable region having the amino acid sequence as shown in SEQ ID NO: 59 and the light chain variable region having the amino acid sequence as shown in SEQ ID NO: 70.

7. The antibody-drug conjugate or the pharmaceutically acceptable salt or solvate thereof according to claim 1, wherein the anti-CDH17 antibody or antigen-binding fragment thereof further comprising human antibody constant regions;
preferably, the heavy chain constant region of the human antibody constant regions is selected from constant regions of human IgG1, IgG2, IgG3 and IgG4 and conventional variants thereof, and the light chain constant region of the human antibody constant regions is selected from κ and λ chain constant regions of human antibody and conventional variants thereof;
more preferably, the full-length antibody comprises a human antibody heavy chain constant region of SEQ ID NO: 99 or having the amino acid sequence of at least 80%, 85%, 90%, 95% or 99% sequence identity therewith, a human light chain constant region of SEQ ID NO: 100 or having the amino acid sequence of at least 80%, 85%, 90%, 95% or 99% sequence identity therewith;
further preferably, the full-length antibody comprises a human antibody heavy chain constant region of SEQ ID NO: 99 and a human light chain constant region of SEQ ID NO: 100.

8. The antibody-drug conjugate or the pharmaceutically acceptable salt or solvate thereof according to claim 1, wherein the anti-CDH17 antibody or antigen-binding fragment thereof comprising:
a heavy chain having the amino acid sequence as shown in SEQ ID NO: 97 or having the amino acid sequence of at least 80%, 85%, 90%, 95% or 99% sequence identity therewith, and a light chain having the amino acid sequence as shown in SEQ ID NO: 98 or having the amino acid sequence of at least 80%, 85%, 90%, 95% or 99% sequence identity therewith; or
a heavy chain having the amino acid sequence as shown in SEQ ID NO: 89 or having the amino acid sequence of at least 80%, 85%, 90%, 95% or 99% sequence identity therewith, and a light chain having the amino acid sequence as shown in SEQ ID NO: 90 or having the amino acid sequence of at least 80%, 85%, 90%, 95% or 99% sequence identity therewith; or
a heavy chain having the amino acid sequence as shown in SEQ ID NO: 85 or having the amino acid sequence of at least 80%, 85%, 90%, 95% or 99% sequence identity therewith, and a light chain having the amino acid sequence as shown in SEQ ID NO: 86 or having the amino acid sequence of at least 80%, 85%, 90%, 95% or 99% sequence identity therewith.

9. The antibody-drug conjugate or the pharmaceutically acceptable salt or solvate thereof according to claim 1, wherein the anti-CDH17 antibody or antigen-binding fragment thereof comprising:
a heavy chain having the amino acid sequence as shown in SEQ ID NO: 97 and a light chain having the amino acid sequence as shown in SEQ ID NO: 98; or
a heavy chain having the amino acid sequence as shown in SEQ ID NO: 89 and a light chain having the amino acid sequence as shown in SEQ ID NO: 90; or
a heavy chain having the amino acid sequence as shown in SEQ ID NO: 85 and a light chain having the amino acid sequence as shown in SEQ ID NO: 86.

10. The antibody-drug conjugate or the pharmaceutically acceptable salt or solvate thereof according to claim 1, wherein the anti-CDH17 antigen-binding fragment is selected from the group consisting of Fab, Fab′, F(ab′)2, variable fragment (Fv), single chain variable fragment (scFv), dimerized domain V (diabody), disulfide stabilized Fv (dsFv) and CDR-containing peptides.

11. The antibody-drug conjugate or the pharmaceutically acceptable salt or solvate thereof according to claim 1, wherein L₁ is selected from the group consisting of:

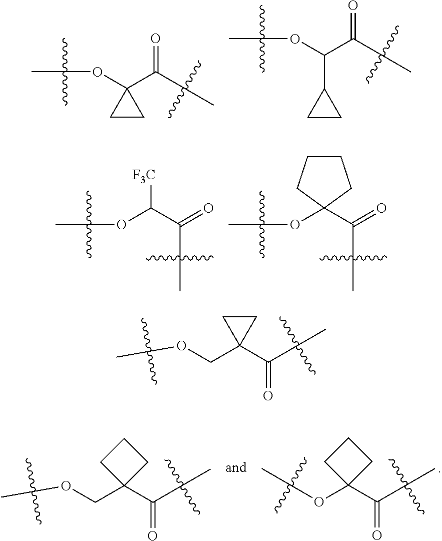

12. The antibody-drug conjugate or the pharmaceutically acceptable salt or solvate thereof according to claim 1, the antibody-drug conjugate is as the following structure: 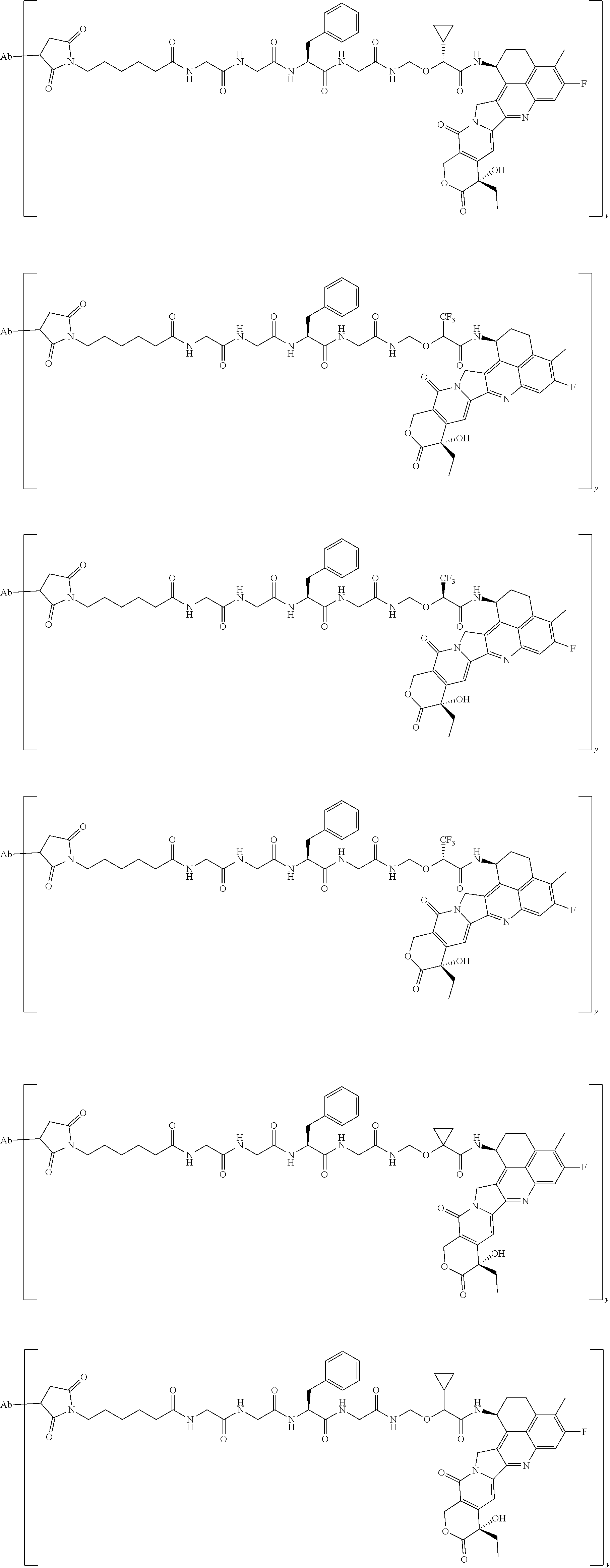 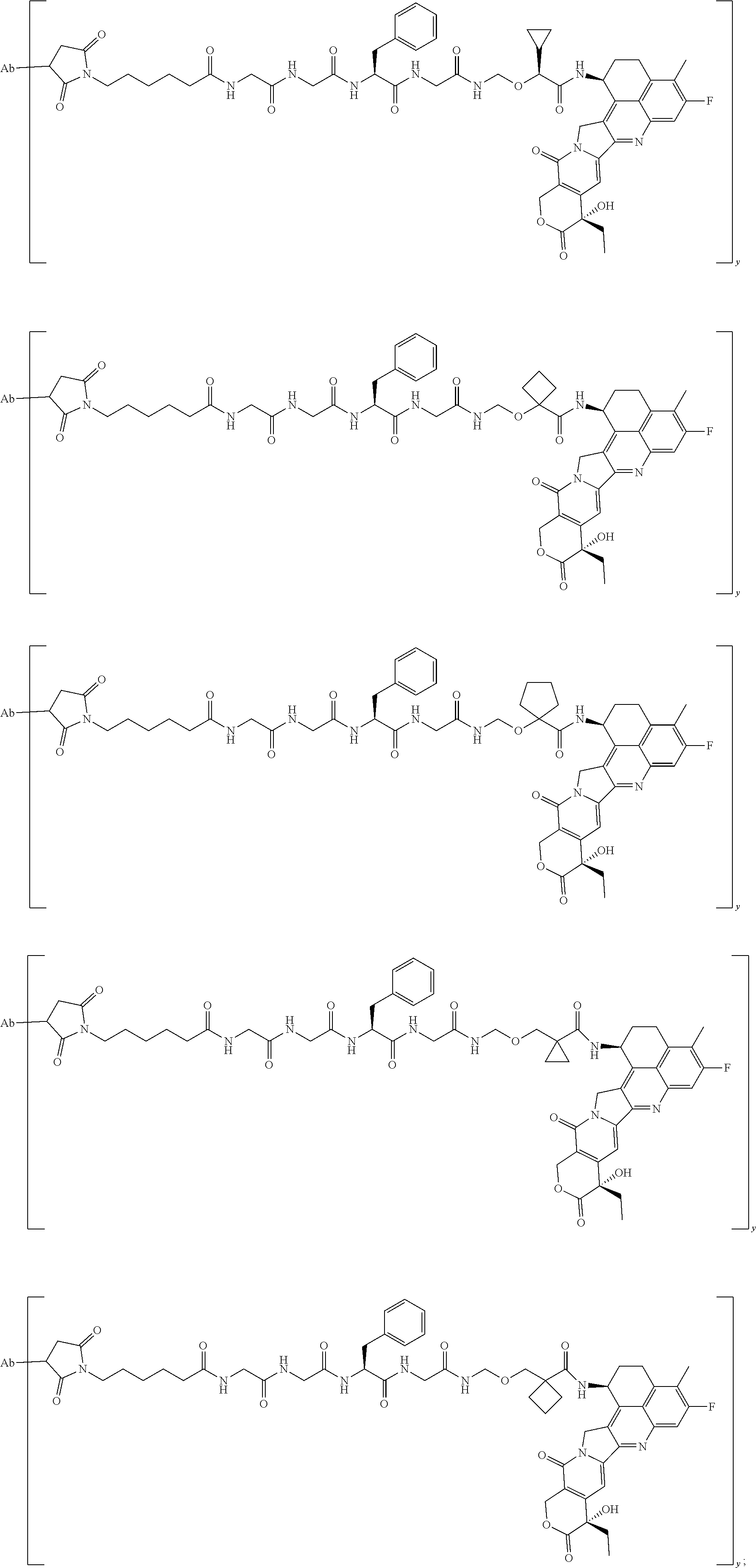
y is a number selected from 1 to 10, preferably a number selected from 2 to 8, and most preferably 2, 4, 5, 6, 7, 8;
Ab is the anti-CDH17 antibody or antigen-binding fragment thereof according to claim 1.

13. The antibody-drug conjugate or the pharmaceutically acceptable salt or solvate thereof according to claim 1, the antibody-drug conjugate is selected from the group consisting of the following compounds: 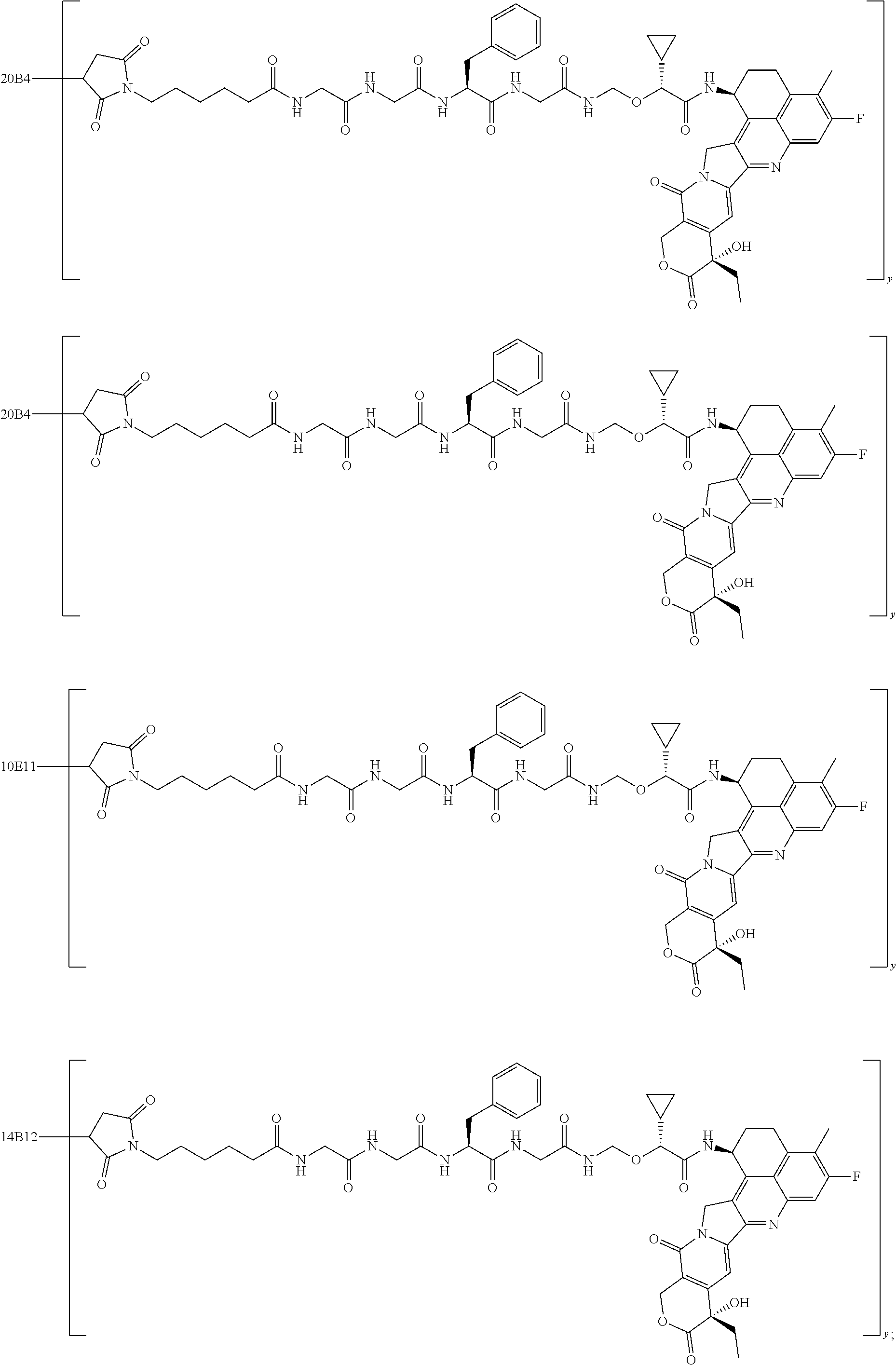
wherein:
y is a number selected from 1 to 10, preferably a number selected from 2 to 8, more preferably a number of 4 to 8, further preferably a number of 4 to 6 or 6 to 8, and most preferably 4, 5, 6, 7 or 8.

14. A pharmaceutical composition comprising the antibody-drug conjugate according to claim 1, or the pharmaceutically acceptable salt or solvate thereof, and one or more pharmaceutically acceptable carriers.

15. A method of treating and/or preventing of a CDH17-mediated disease or condition in a subject in need thereof, wherein the method comprises the step of administering to the subject the antibody-drug conjugate or the pharmaceutically acceptable salt or solvate thereof according to claim 1, or a pharmaceutical composition thereof.

16. The method according to claim 15, wherein the CDH17-mediated disease or condition is a tumor or cancer.

17. The method according to claim 16, wherein the tumor or cancer is associated with moderate or high CDH17 expression.

18. A method of treating and/or preventing a tumor or cancer in a subject in need thereof, wherein the method comprises the step of administering to the subject the antibody drug conjugate or the pharmaceutically acceptable salt or solvate thereof according to claim 1, or a pharmaceutical composition thereof.

19. The method according to claim 18, wherein the tumor or cancer is gastrointestinal cancer, pancreatic cancer, gallbladder cancer, cholangiocarcinoma, stomach cancer, intestinal cancer, ovarian cancer, colorectal cancer, lung cancer, breast cancer, anal cancer, prostate cancer, kidney cancer, bladder cancer, pharynx cancer, cancer of the nose, skin cancer, oral cancer, cancer of the tongue, esophageal cancer, vaginal cancer, cervical cancer, cancer of the spleen, testicular cancer, or glioblastoma.
